(19) United States
(12) Patent Application Publication
(10) Pub. No.: US 2020/0181216 A1
(43) Pub. Date: Jun. 11, 2020

(54) HSA-GDF-15 FUSION POLYPEPTIDE AND USE THEREOF

(71) Applicant: NOVARTIS AG, Basel (CH)

(72) Inventors: Rajiv CHOPRA, Andover, MA (US); Norio HAMAMATSU, Belmont, MA (US); Ryan STREEPER, Needham, MA (US); Brian Edward VASH, Cambridge, MA (US)

(21) Appl. No.: 16/541,703

(22) Filed: Aug. 15, 2019

Related U.S. Application Data
(63) Continuation of application No. 15/321,246, filed on Dec. 22, 2016, now abandoned.
(60) Provisional application No. 62/015,945, filed on Jun. 23, 2014.

(51) Int. Cl. C07K 14/495; C07K 14/765; C07K 14/475

(52) U.S. Cl. CPC C07K 14/495; A61K 38/00; C07K 14/4756; C07K 14/765

(57) ABSTRACT

The disclosure relates to fusion polypeptides comprising serum albumin or a functional variant thereof and GDF15 protein or a functional variant thereof, and to pharmaceutical compositions that contain the fusion polypeptides, nucleic acids that encode the fusion polypeptides, methods of making the polypeptides and use of the polypeptides to decreasing appetite, decreasing body weight and treating metabolic diseases.

### BACKGROUND OF THE INVENTION

Obesity has reached near epidemic proportions, with an estimated 36% of the adult population considered obese or overweight. Obesity is a chronic disease associated with high morbidity and mortality. Obesity presents its own health problems, and is also associated with a variety of other diseases such as hypertension, hyperlipidemia, diabetes mellitus, atherosclerosis, coronary artery disease, sleep apnea, gout, rheumatism and arthritis. About 80% of obese patients have the one or more of the above diseases (Mantzoros et al., J Clin Endocrinol Metab 2000; 85:4000-2), and approximately 300,000 people die each year due to complications from obesity (Allison et al., JAMA 1999; 282:1530-8). A weight gain of just 1 kg has been shown to increase cardiovascular risk by 3.1% and diabetes risk by 4.5-9%, and a weight loss of about 11% has been shown to reduce morbidity by 25%.

While most people can diet and lose weight, durable weight loss can be difficult to maintain, as calorie restriction results in activation of the hypothalamic neurons that promote food intake and weight regain. Therefore, many turn to surgical and/or medical approaches to achieve durable weight loss. However, surgical and medical therapies for obesity have limited efficacy and significant side effects. For example, bariatric surgery is a major surgical procedure with considerable risk of complications, and requires extensive lifestyle modification. Drug therapy for obesity (e.g., using phentermine and or topiramate) has limited efficacy and is further limited by side-effects.

Growth Differentiation Factor 15 (GDF15) is a divergent member of the TGFβ superfamily, and is also referred to as macrophage inhibitory cytokine 1 (MIC1) (Bootcov MR, 1997, Proc Natl Acad Sci 94: 11514-9), placental bone morphogenetic factor (PLAB) (Hromas R 1997, Biochim Btophys Acta. 1354:40-4), placental transforming growth factor beta (PTGFB) (Lawton L N 1997, Gene. 203: 17-26), prostate derived factor (PDF) (Paralkar V M 1998, J Biol Chem. 273: 13760-7), and nonsteroidal antiinflammatory drug-activated gene (NAG-1) (Baek S J 2001, J Biol Chem. 276: 33384-92). The mature GDF15 peptide shares low homology with other family members (Katoh M 2006, Int J Mol Med. 17:951-5). GDF15 is synthesized as a large precursor protein that is cleaved at the dibasic cleavage site to release the carboxyterminal mature peptide. Human full-length precursor contains 308 amino acids and is cleaved at the RGRRRAR (SEQ ID NO:43) cleavage site to produce the mature GDF peptide. Naturally occurring GDF15 is a 25 KD homodimer of the mature peptide covalently linked by one inter-chain disulfide bond.

GDF15 is reported to be relevant to a number of different physiological and pathologic conditions. For example, studies of GDF15 knockout and transgenic mice suggest that GDF15 may be protective against ischemic/reperfusion- or overload-induced heart injury (Kempf T, 2006, Circ Res. 98:351-60) (Xu J, 2006, Circ Res. 98:342-50), protective against aging-associated motor neuron and sensory neuron loss (Strelau J, 2009, J Neurosci. 29: 13640-8), mildly protective against metabolic acidosis in kidney, and may cause cachexia in cancer patients (Johnen H 2007 Nat Med. 11: 1333-40). GDF15 is also reported to be protective against carcinogen- or Apc mutation-induced neoplasia in intestine and lung (Baek S J 2006, Gastroenterology. 131: 1553-60; Cekanova M 2009, Cancer Prev Res 2:450-8).

GDF15 has anorexigenic effects, particularly in cancer (Brown D. A. Clinical Cancer Res 2003; 9:2642-2650; Koopmann J. Clinical Cancer Res 2006; 12:442-446). Substantial elevation of circulating MIC-1/GDF15 levels in cancers and other diseases such as chronic renal or cardiac failure are associated with a lower body mass index (Breit S. N. et al, Growth factors 2011; 29:187-195; Johnen H. et al, Nat Med. 2007; 13:1333-1340), suggesting that apart from any role in inflammation in disease, MIC-1/GDF15 may also play a role in body weight regulation. Long-term elevated expression of MIC-1/GDF15 in mice leads to decreased food intake, body weight and adiposity with concomitantly improved glucose tolerance, both under normal and obesogenic dietary conditions (Macia L. et al, PloS One 2012; 7(4):e34868). Food intake and body weight are controlled by a variety of central and peripheral factors, but the exact mechanisms behind these processes are still not fully understood.

Human Serum Albumin (HSA) is a plasma protein of about 66,500 KDa and is comprised of 585 amino acids, including at least 17 disulfide bridges. (Peters, T., Jr. (1996), All about Albumin: Biochemistry, Genetics and Medical, Applications, pp 10, Academic Press, Inc., Orlando (ISBN 0-12-552110-3). HSA has a long half-life and is cleared very slowly by the liver. The plasma half-life of HSA is reported to be approximately 19 days (Peters, T., Jr. (1985) Adv. Protein Chem. 37, 161-245; Peters, T., Jr. (1996) All about Albumin, Academic Press, Inc., San Diego, Calif. (page 245-246)); Benotti P, Blackburn G L: Crit Care Med (1979) 7:520-525).

HSA has been used to produce fusion proteins that have improved shelf and half-lifes. For example, PCT Publications WO 01/79271 A and WO 03/59934 A disclose a albumin fusion proteins comprising a variety of therapeutic protein (e.g., growth factors, scFvs) and HSA that are reported to have longer shelf and half-lifes than the therapeutic proteins alone.

PCT Publication WO 13/113008 A discloses GDF15-Fc fusions for treatment or amelioration of metabolic disorders including obesity. This patent application reports efficacy of GDF15-Fc fusion in obese mice and overweight monkeys.

There is a need for new therapeutic agents for the treatment of obesity. (See, e.g., Arbeeny et al., Obes Res 2004; 12:1191-6). There is a particular need for improved GDF15 fusion proteins that are active and have improved therapeutic properties, such as a longer serum half-life than naturally occurring GDF15 and that are stable.

### SUMMARY OF THE INVENTION

The present invention relates to fusion polypeptides comprising the Human Serum Albumin (HSA) or a functional variant thereof and the human GDF15 or a functional variant thereof.

The fusion polypeptides comprise a first moiety, a second moiety and optionally a linker that links the first moiety to the second moiety. The first moiety can be human serum albumin (HSA) or a functional variant thereof, the second moiety is human GDF15 protein or a functional variant thereof; and the first moiety is amino terminal to the second moiety.

The first moiety can have at least about 80% sequence identity to mature HSA (SEQ ID NO:45). For example, the first moiety can be mature HSA (SEQ ID NO:45). In other examples, the first moiety is a functional variant of HSA, such as a portion of HSA as described herein, or mature HSA in which one or more amino acids is replaced with another amino (e.g., C34S and N503Q).

In some aspects, the fusion polypeptides contains a first moiety is selected from the group consisting of HSA (25-609) (SEQ ID NO:45), and HSA(25-609) in which Cys34 is replaced with Ser and Asn503 is replaced with Gin; and a second moiety is selected from the group consisting human mature GDF15 peptide (197-308) (SEQ ID NO:44), human GDF15(211-308) (amino acids 211-308 of SEQ ID NO:1), human GDF15(197-308) (SEQ ID NO:44) in which Cys203 is replaced with Ser (C203S) and Cys210 is replaced with Ser (C210S), human GDF15(97-308) (SEQ ID NO:44) in which Cys273 is replaced with Ser (C273S).

In some fusion polypeptides, such as those in which the first moiety is mature HSA (SEQ ID NO:45) the second moiety includes a functional variant of GDF15 (SEQ ID NO:44), such as a variant in which the amino acid residue in the GDF15 protein or a functional variant thereof that corresponds to position 198 of SEQ ID NO: 1 is not Arg, the amino acid residue in the GDF15 protein or a functional variant thereof that corresponds to position 199 of SEQ ID NO: 1 is not Asn; or the amino acid residue in the GDF15 protein or a functional variant thereof that corresponds to position 198 of SEQ ID NO: 1 is not Arg and the amino acid residue that corresponds to position 199 of SEQ ID NO:1 is not Asn. In a particular example, the fusion polypeptides contains a second moiety in which the amino acid that corresponds to position 198 in human GDF15 is His and amino acid that corresponds to position 199 in human GDF15 is Ala.

If desired, the second moiety in the fusion polypeptide can additionally or alternatively comprises an amino acid replacement or deletion of one or more surface exposed residues, one or more N-terminal amino acids (amino acids 197-210), Cys 203, Cys 210 and/or Cys273. Amino acid residues that are surface exposed on GDF15 include Arg217, Ser219, Ala226, Glu234, Ala243, Ser246, Gin247, Arg263, Lys265, Thr268, Ala277, Asn280, Lys287, Thr290, Lys303 and Asp304.

In certain aspects the fusion polypeptides further comprises a linker that links the first moiety and the second moiety. For example, the linker can be sequence selected from the group consisting of (GGGGS)n and (GPPGS)n, wherein n is one to about 20. In particular embodiments, the linker is (GGGGS)n, and n is 3.

In more particular embodiments, the fusion polypeptide has an amino acid sequence selected from the group consisting of SEQ ID NOS:20, 26, 28, 30, 32, 38, 40 and 42. The fusion polypeptide can be a homodimer, heterodimer or monomer, and is preferably a homodimer or monomer.

In other aspects, the invention relates to a nucleic acid molecule (e.g., an isolated nucleic acid molecule), including DNA and RNA molecule and expression vectors, that encodes a fusion polypeptide as described herein. The invention also relates to a host cell comprising a recombinant nucleic acid that encodes a fusion polypeptide as described herein. The invention also relates to a method for making an a fusion polypeptide as described herein, comprising maintaining a host cell of the invention under conditions suitable for expression of the nucleic acid, whereby the recombinant nucleic acid is expressed and the fusion polypeptide is produced. If desired, the method can further comprise isolating the fusion polypeptide.

The invention also relates to a pharmaceutical composition comprising a fusion polypeptide as described herein and a pharmaceutically or physiologically acceptable carrier. Preferred pharmaceutical compositions are for subcutaneous administration.

The invention also relates to methods for decreasing appetite, decreasing body weight and treating metabolic diseases in a subject in need thereof, said method comprising administering to the subject in need thereof an effective amount of a GDF15 fusion polypeptide (usually in the form of a pharmaceutical composition) as described herein. In some aspects, the invention relates to methods for treating type 2 diabetes mellitus, obesity, pancreatitis, dyslipidemia, nonalcoholic steatohepatitis, insulin resistance, hyperinsulinemia, glucose intolerance, hyperglycemia, metabolic syndrome, hypertension, cardiovascular disease, atherosclerosis, peripheral arterial disease, stroke, heart failure, coronary heart disease, diabetic complications (including but not limited to chronic kidney disease), neuropathy, gastroparesis and other metabolic disorders or body weight disorders in a subject in need thereof, said method comprising administering to the subject in need thereof an effective amount of a GDF15 fusion polypeptide (usually in the form of a pharmaceutical composition) as described herein. In particular aspects, the invention relates to a methods for treating genetic obesity in a subject in need thereof, such as a subject with Prader-Willi syndrome, leptin mutations and/or melanocortin 4 receptor mutations, said method comprising administering to the subject in need thereof an effective amount of a GDF15 fusion polypeptide (usually in the form of a pharmaceutical composition) as described herein.

The invention also relates to the use of a fusion polypeptide as described herein for use in therapy and in the manufacture of a medicament for treating a disease or condition as disclosed herein (e.g., decreasing appetite, decreasing body weight and treating metabolic diseases).

### BRIEF DESCRIPTION OF THE DRAWINGS

FIGS. 1a and 1b are images of polyacrylamide gels in which Fc-GDF15 fusion protein (FIG. 1a) or mouse serum Albumin-GDF15 fusion proteins (FIG. 1b) were run under non-reducing and reducing conditions. FIG. 1a shows that a large proportion of the fusion protein (SEQ ID NO:36) migrated close to the origin under non-reducing conditions, indicating that the fusion protein aggregated. FIG. 1b. shows that the albumin fusion protein (SEQ ID NO: 16) migrated at the expected molecular weight under non-reducing conditions, indicating that the fusion protein did not aggregate.

### DETAILED DESCRIPTION OF THE INVENTION

The invention relates to GDF15 fusion polypeptides and to the use of such fusion polypeptides to decrease appetite, promote weight loss, and treat obesity and other metabolic diseases. The GDF15 fusion polypeptides are contiguous polypeptide chains that include a GDF15 moiety and a serum albumin (SA) moiety. The SA and GDF15 moieties can be directly bonded to each other in the contiguous polypeptide chain, or preferably indirectly bonded to each other through a suitable linker.

The present application describes the determination of the X-ray crystal structure of the human mature GDF15 protein, incorporating amino-acids 197-308 of SEQ ID NO: 1. The crystal structure reveals a disulfide-linked dimeric structure. Each GDF15 monomer adopts a fold similar to other TGFbeta superfamily cysteine knot proteins with a significant difference seen at the N-terminal. The mature GDF15 protein contains a total of nine cysteines all of which are disulfide bonded with Cys273, forming the inter-chain disulfide across the dimer interface. The disulfide bonding pattern of the first four Cysteines is unique to GDF15 when compared with TGFbeta and BMP family members. Cys203 and Cys210 (the first two cysteines in the mature protein) form a disulfide with each other to make a small loop structure protruding from the protein.

The remaining disulfides are structurally similar to the TGFbeta family but are formed by Cys211-Cys274 (third and seventh cysteines), Cys240-Cys305 (fourth and eighth cysteines) and Cys244-Cys307 (fifth and ninth cysteines). The crystal structure further revealed that there is an extensive peptide-peptide interface in the human GDF-15 homodimer, with ˜1300 square Angstroms of buried surface area and involvement of 37 amino acids. The crystal structure shows that the following amino acids are involved in the peptide-peptide interface: Val216, Asp222, Leu223, Trp225, Val237, Met239, Ile241, Asn252, Met253, His254, Ile257, Lys258, Ser260, Leu261, Leu264, Lys265, Thr268, Val269, Pro270, Cys273, Val275, Pro276, Tyr279, Tyr297, Asp299, Leu300 and Ile308. The last amino-acid of the mature peptide, Ile308, is positioned fewer than 10 angstroms away from its dimer partner. Unusually for the superfamily, the electron density is consistent with the side-chain pointing toward the interior of the protein structure to form a hydrophobic pocket with Val275 and Pro276. Other family members have the carboxylic acid pointing toward the inside of the structure and the sidechain solvent exposed (ref TGFb3 (2PJY), BMP6(2R52), BMP7(1LX5), GDF5(3EVS), GDF2(4FAO)). This suggests that GDF15 might be unique in its ability to accommodate longer peptide sequences at the COOH-termini without perturbation of its protein fold.

Utilizing the crystal structure residues forming the functional epitope responsible for receptor recruitment and subsequent signaling were identified as those comprising either the Fingers domain, knuckle domain, wrist domain, the newly discovered N-terminal domain, Carboxy-terminal domain or back-of-hand domain. Further, it was recognized that the addition of a fusion protein would be required to not interfere, directly or indirectly, with either the folding of the protein dimer nor with the functional epitope. A series of structure-guided site-directed mutants were designed to identify a) domains and residues whose alteration adversely affected GDF15 function and b) domains and residues amenable to modification. (See, exemplification) From these studies, the knuckle domain was identified as being critical for function and the N-terminal domain, wrist domain, fingers domain, and back of hand domain were identified as potential sites for modification. It was determined that GDF15 fusion polypeptides in which a fusion partner is fused to the C-terminus or C-terminally to GDF15 are not effective in causing weight loss. In contrast, GDF15 fusion polypeptides in which a fusion partner (e.g., SA) is fused to the N-terminus or N-terminally to GDF15 have weight loss activity and were effective in causing weight loss in model systems. (See, exemplification). Accordingly, in the GDF15 fusion polypeptides disclosed herein, the SA portion is located at the N-terminus, or N-terminally to the GDF15 portion.

The fusion polypeptides described herein can contain any suitable SA moiety, any suitable GDF15 moiety, and if desired, any suitable linker. Generally, the SA moiety, GDF15 moiety and, if present, linker are selected to provide a fusion polypeptide that has weight loss activity (e.g., in vivo) and to be immunologically compatible with the species to which it is intended to be administered. For example, when the fusion polypeptide is intended to be administered to humans the SA moiety can be HSA or a functional variant thereof, and the GDF15 moiety can be human GDF15 or a functional variant thereof. Similarly, SA and functional variants thereof and GDF15 and functional variants thereof that are derived from other species (e.g., pet or livestock animals) can be used when the fusion protein is intended for use in such species.

### GDF15 Moiety

The GDF15 moiety is any suitable GDF15 polypeptide or functional variant thereof. Preferably, the GDF15 moiety is human GDF15 or a functional variant thereof. Human GDF15 is synthesized as a 308 amino acid preproprotein (SEQ ID NO: 1) that includes a signal peptide (amino acids 1-29), a propeptide (amino acids 30-196), and the 112 amino acid mature GDF15 peptide (amino acids 197-308 (SEQ ID NO:44)). The propeptide and mature peptide have been reported as amino acids 30-194 and 195-308 of SEQ ID NO: 1, respectively. (See, Uniprot sequence Q99988.) Sequence variations have been reported. For example, amino acids 202, 269 and 288 (in SEQ ID NO: 1) have been reported to be Asp, Glu and Ala, respectively. (Hromas R, et al., Biochem. Biophys. Acta 1354:40-44 (1997), Lawton L. N. et al. Gene 203:17-26 (1997).)

Fusion proteins of the present invention that contain a human GDF15 moiety generally contain the 112 amino acid mature GDF15 peptide (e.g., amino acids 197-308 of SEQ ID NO: 1, SEQ ID NO:44) or a functional variant thereof. The functional variant can include one or more amino acid deletions, additions or replacements in any desired combination. The amount of amino acid sequence variation (e.g., through amino acid deletions, additions or replacements) is limited to preserve weight loss activity of the mature GDF15 peptide. In some embodiments, the functional variant of a mature GDF15 peptide has from 1 to about 20, 1 to about 18, 1 to about 17, 1 to about 16, 1 to about 15, 1 to about 14, 1 to about 13, 1 to about 12, 1 to about 11, 1 to about 10, 1 to about 9, 1 to about 8, 1 to about 7, 1 to about 6, or 1 to about 5 amino acid deletions, additions or replacements, in any desired combination, relative to SEQ ID NO:44. Alternatively or in addition, the functional variant can have an amino acid sequence that has at least about 80%, at least about 85%, at least about 90%, or at least about 95% amino acid sequence identity with SEQ ID NO:44, preferably when measured over the full length of SEQ ID NO:44.

Without wishing to be bound by any particular theory, it has been suggested that GDF15 weight loss activity is mediated through cellular signaling initiated by the binding of GDF15 (and the fusion polypeptides described herein) to one or more receptors. While no receptor binding studies have been reported for GDF15, it is believed that GDF15 binds to and activates signaling through the Transforming Growth Factor Beta Type II receptor (TGFBR2). Accordingly, when the fusion polypeptide contains a functional variant of GDF15, any amino acid deletions, additions or replacements are preferably at positions that are not involved with receptor binding or with the intra-peptide interface and amino acid replacements are preferably conservative replacements. For example, the amino acids at positions 216, 222, 223, 225, 237, 239, 241, 252, 253, 254, 257, 258, 260, 261, 264, 265, 268, 269, 270, 273, 275, 276, 279, 297, 299, 300 and 308 are involved in the peptide-peptide interface. Any amino acid replacements at these positions are generally disfavored, and any replacements should be conservative replacements. Amino acids that are surface exposed but are not conserved among species can generally be replaced with other amino acids without disrupting the folding of the peptide or its weight loss activity. The inventors have determined the crystal structure of the human mature GDF15 peptide and identified the amino acids at positions 217, 219, 226, 234, 243, 246, 247, 263, 265, 268, 277, 280, 287, 290, 303 and 304 as surface exposed residues that are not conserved in other species. In addition, the amino terminal of mature human GDF15 (amino acids 197-210 of SEQ ID NO: 1) and Cys203, Cys 210 and Cys273, which are not essential for weight loss activity, can generally be replaced with another amino acid and/or omitted.

Exemplary variants of human mature GDF15 peptide that are suitable for use in the fusion polypeptides include SEQ ID NO:44 in which one or more of the residues from position 1 to about 25 are replaced or deleted. For example, the variant can have the sequence of SEQ ID NO:44 in which the first 25, the first 15, the first 14, the first 13, the first 12, the first 11, the first 10, the first 9, the first 8, the first 7, the first 6, the first 5, the first 4, the first 3, the first 2, or the first 1 amino acid is deleted.

Additional exemplary variants of human mature GDF15 peptide that are suitable for use in the fusion polypeptides of the present invention include amino acids 197-308 of SEQ ID NO: 1 (SEQ ID NO:44) in which the Arg at position 198, Asn at position 199, or Arg at position 198 and Asn at position 199 are replaced with one or more other amino acids. When amino acids are replaced, conservative amino acid replacements are preferred. In particular embodiments, Arg at position 198 is replaced with His or Gly at position 199 is replaced with Ala or Glu. In more particular embodiments Arg at position 198 is replaced with His and Asn at position 199 is replaced with Ala.

Mature human GDF15 includes 9 cysteine residues, eight of which form intra-chain disulfide bonds in a pattern that is unique among TGFbeta superfamily members. Cys203, 210 and 273 are not required for weight loss activity and can be replaced with other amino acids or omitted if desired. Mutations of other cysteines in mature human GDF15 resulted in decreased or lost activity.

### SA Moiety

The SA moiety is any suitable serum albumin (e.g., human serum albumin (HSA), or serum albumin from another species) or a functional variant thereof. Preferably, the SA moiety is an HSA or a functional variant thereof. The SA moiety prolongs the serum half-life of the fusion polypeptides to which it is added, in comparison to wild type GDF15. Methods for pharmacokinetic analysis and determination of serum half-life will be familiar to those skilled in the art. Details may be found in Kenneth. A et al: Chemical Stability of Pharmaceuticals: A Handbook for Pharmacists and in Peters et al. Pharmacokinetc analysis: A Practical Approach (1996). Reference is also made to “Pharmacokinetics,” M Gibaldi & D Perron, published by Marcel Dekker, 2^{nd} Rev. ex edition (1982), which describes pharmacokinetic parameters such as t alpha and t beta half-lives and area under the curve (AUC).

HSA may comprise the full length sequence of 585 amino acids of mature naturally occurring HSA (following processing and removal of the signal and propeptides (SEQ ID NO:45)) or naturally occurring variants thereof, including allelic variants. Naturally occurring HSA and variants thereof are well-known in the art. (See, e.g., Meloun, et al., FEBS Letters 58:136 (1975); Behrens, et al., Fed. Proc. 34:591 (1975); Lawn, et al., Nucleic Acids Research 9:6102-6114 (1981); Minghetti, et al., J. Biol. Chem. 261:6747 (1986)); and Weitkamp, et al., Ann. Hum. Genet. 37:219 (1973).)

Fusion proteins that contain a human serum albumin moiety generally contain the 585 amino acid HSA (amino acids 25-609 of SEQ ID NO:2, SEQ ID NO:45) or a functional variant thereof. The functional variant can include one or more amino acid deletions, additions or replacement in any desired combination, and includes functional fragments of HSA. The amount of amino acid sequence variation (e.g., through amino acid deletions, additions or replacements) is limited to preserve the serum half-life extending properties of HSA.

In some embodiments, the functional variant of HSA for use in the fusion proteins disclosed herein can have an amino acid sequence that has at least about 80%, at least about 85%, at least about 90%, or at least about 95% amino acid sequence identity with SEQ ID NO:45, preferably when measured over the full length sequence of SEQ ID NO:45. Alternatively or in addition, the functional variant of HSA can have from 1 to about 20, 1 to about 18, 1 to about 17, 1 to about 16, 1 to about 15, 1 to about 14, 1 to about 13, 1 to about 12, 1 to about 11, 1 to about 10, 1 to about 9, 1 to about 8, 1 to about 7, 1 to about 6, or 1 to about 5 amino acid deletions, additions or replacement, in any desired combination.

Some functional variants of HSA for use in the fusion proteins disclosed herein may be at least 100 amino acids long, or at least 150 amino acids long, and may contain or consist of all or part of a domain of HSA, for example domain I (amino acids 1-194 of SEQ ID NO:45), II (amino acids 195-387 of SEQ ID NO:45), or III (amino acids 388-585 of SEQ ID NO:45). If desired, a functional variant of HSA may consist of or alternatively comprise any desired HSA domain combination, such as, domains I+II (amino acids 1-387 of SEQ ID NO:45), domains II+III (amino acids 195-585 of SEQ ID NO:45) or domains I+III (amino acids 1-194 of SEQ ID NO:45+amino acids 388-585 of SEQ ID NO:45). As is well-known in the art, each domain of HSA is made up of two homologous subdomains, namely amino acids 1-105 and 120-194, 195-291 and 316-387, and 388-491 and 512-585 of domains I, II, and III respectively, with flexible inter-subdomain linker regions comprising residues Lys106 to Glu119, Glu292 to Val315 and Glu492 to Ala511. In certain embodiments, the SA moiety of the fusions proteins of the present invention contains at least one subdomain or domain of HSA.

Functional fragments of HSA suitable for use in the fusion proteins disclosed herein will contain at least about 5 or more contiguous amino acids of HSA, preferably at least about 10, at least about 15, at least about 20, at least about 25, at least about 30, at least about 50, or more contiguous amino acids of HSA sequence or may include part or all of specific domains of HSA.

In some embodiments, the functional variant (e.g., fragment) of HSA for use in the fusion proteins disclosed herein includes an N-terminal deletion, a C-terminal deletions or a combination of N-terminal and C-terminal deletions. Such variants are conveniently referred to using the amino acid number of the first and last amino acid in the sequence of the functional variant. For example, a functional variant with a C-terminal truncation can be amino acids 1-387 of HSA (SEQ ID NO:45).

Examples of HSA and HSA variants (including fragments) that are suitable for use in the GDF15 fusion polypeptides described herein are known in the art. Suitable HSA and HSA variants include, for example full length mature HSA (SEQ ID NO:45) and fragments, such as amino acids 1-387, amino acids 54 to 61, amino acids 76 to 89, amino acids 92 to 100, amino acids 170 to 176, amino acids 247 to 252, amino acids 266 to 277, amino acids 280 to 288, amino acids 362 to 368, amino acids 439 to 447, amino acids 462 to 475, amino acids 478 to 486, and amino acids 560 to 566 of mature HSA. Such HSA polypeptides and functional variants are disclosed in PCT Publication WO 2005/077042A2, which is incorporated herein by reference in its entirety. Further variants of HSA, such as amino acids 1-373, 1-388, 1-389, 1-369, 1-419 and fragments that contain amino acid 1 through amino acid 369 to 419 of HSA are disclosed in European Published Application EP322094A1, and fragments that contain 1-177, 1-200 and amino acid 1 through amino acid 178 to 199 are disclosed in European Published Application EP399666A1.

### Linkers

The SA and GDF15 moieties described in this invention can be directly bonded to each other in the contiguous polypeptide chain, or preferably indirectly bonded to each other through a suitable linker. The linker is preferably a peptide linker. Peptide linkers are commonly used in fusion polypeptides and methods for selecting or designing linkers are well-known. (See, e.g., Chen X et al. Adv. Drug Deliv. Rev. 65(10):135701369 (2013) and Wriggers W et al., Biopolymers 80:736-746 (2005).)

Peptide linkers generally are categorized as i) flexible linkers, ii) helix forming linkers, and iii) cleavable linkers, and examples of each type are known in the art. Preferably, a flexible linker is included in the fusion polypeptides described herein. Flexible linkers may contain a majority of amino acids that are sterically unhindered, such as glycine and alanine. The hydrophilic amino acid Ser is also conventionally used in flexible linkers. Examples of flexible linkers include, polyglycines (e.g., (Gly)₄ and (Gly)₅), polyalanines poly(Gly-Ala), and poly(Gly-Ser) (e.g., (Glyₙ-Serₙ)ₙ or (Serₙ-Glyₙ)ₙ, wherein each n is independent an integer equal to or greater than 1).

Peptide linkers can be of a suitable length. The peptide linker sequence may be at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more amino acid residues in length. For example, a peptide linker can be from about 5 to about 50 amino acids in length; from about 10 to about 40 amino acids in length; from about 15 to about 30 amino acids in length; or from about 15 to about 20 amino acids in length. Variation in peptide linker length may retain or enhance activity, giving rise to superior efficacy in activity studies. The peptide linker sequence may be comprised of a naturally, or non-naturally, occurring amino acids.

In some aspects, the amino acids glycine and serine comprise the amino acids within the linker sequence. In certain aspects, the linker region comprises sets of glycine repeats (GSG₃)ₙ, where n is a positive integer equal to or greater than 1 (preferably 1 to about 20) (SEQ ID NO:50). More specifically, the linker sequence may be GSGGG (SEQ ID NO:51). The linker sequence may be GSGG (SEQ ID NO:52). In certain other aspects, the linker region orientation comprises sets of glycine repeats (SerGly₃)ₙ, where n is a positive integer equal to or greater than 1 (preferably 1 to about 20) (SEQ ID NO:53).

In more preferred embodiments, a linker may contain glycine (G) and serine (S) in a random or preferably a repeated pattern. For example, the linker can be (GGGGS)ₙ (SEQ ID NO:46), wherein n is an integer ranging from 1 to 20, preferably 1 to 4. In a particular example, n is 3 and the linker is GGGGSGGGGSGGGGS (SEQ ID NO:47).

In other preferred embodiments, a linker may contain glycine (G), serine (S) and proline (P) in a random or preferably repeated pattern. For example, the linker can be (GPPGS)ₙ (SEQ ID NO:48), wherein n is an integer ranging from 1 to 20, preferably 1-4. In a particular example, n is 1 and the linker is GPPGS (SEQ ID NO:49).

In general, the linker is not immunogenic when administered in a patient, such as a human. Thus linkers may be chosen such that they have low immunogenicity or are thought to have low immunogenicity.

The linkers described herein are exemplary, and the linker can include other amino acids, such as Glu and Lys, if desired. The peptide linkers may include multiple repeats of, for example, (G₄S) (SEQ ID NO:54), (G₃S) (SEQ ID NO:55), (G₂S) (SEQ ID NO:56) and/or (GlySer) (SEQ ID NO:57), if desired. In certain aspects, the peptide linkers may include multiple repeats of, for example, (SG₄) (SEQ ID NO:58), (SG₃) (SEQ ID NO:59), (SG₂) (SEQ ID NO:60) or (SerGly) (SEQ ID NO:51). In other aspects, the peptide linkers may include combinations and multiples of repeating amino acid sequence units, such as (G₃S)+(G₄S)+(GlySer) (SEQ ID NO:55+SEQ ID NO:54+SEQ ID NO:57). In other aspects, Ser can be replaced with Ala e.g., (G₄A) (SEQ ID NO:62) or (G₃A) (SEQ ID NO:63). In yet other aspects, the linker comprises the motif (EAAAK)ₙ, where n is a positive integer equal to or greater than 1, preferably 1 to about 20. (SEQ ID NO:64) In certain aspects, peptide linkers may also include cleavable linkers.

### GDF15 Fusion Polypeptides

The GDF15 fusion polypeptides described herein contain a GDF15 moiety and an SA moiety, and optionally a linker. The fusion polypeptide is a contiguous amino acid chain in which the SA moiety is located N-terminally to the GDF15 moiety. The C-terminus of the SA moiety can be directly bonded to the N-terminus of the GDF15 moiety. Preferably, the C-terminus of the SA moiety is indirectly bonded to the N-terminus of the GDF15 moiety through a peptide linker.

The SA moiety and GDF15 moiety can be from any desired species. For example, the fusion protein can contain SA and GDF15 moieties that are from human, mouse, rat, dog, cat, horse or any other desired species. The SA and GDF15 moieties are generally from the same species, but fusion peptides in which the SA moiety is from one species and the GDF15 moiety is from another species (e.g., mouse SA and human GDF15) are also encompassed by this disclosure.

In some embodiments, the fusion polypeptide comprises mouse serum albumin or functional variant thereof and mature human GDF15 peptide or functional variant thereof. For example, the fusion protein can have the amino acid sequence of any of SEQ ID NOS: 16, 18, 22, 24 and 34.

In preferred embodiments, the SA moiety is an HSA or a functional variant thereof and the GDF15 moiety is the mature human GDF peptide or a functional variant thereof. When present, the optional linker is preferably a flexible peptide linker. In particular embodiments, the fusion polypeptide comprises

A) an SA moiety selected from the group consisting of HSA(25-609) (SEQ ID NO:45), and HSA(25-609) in which Cys34 is replaced with Ser and Asn503 is replaced with Gin; and

B) a GDF15 moiety selected from the group consisting of:
human GDF15(197-308) (SEQ ID NO:44);
human GDF15(211-308) (amino acids 211-308 of SEQ ID NO: 1);
human GDF15(197-308) (SEQ ID NO:44) in which Cys203 is replaced with Ser (C203S) and Cys210 is replaced with Ser (C210S); and
human GDF15(197-308) (SEQ ID NO:44) in which Cys273 is replaced with Ser (C273S).

If desired, the fusion polypeptide can further comprise a linker that links the C-terminus of the SA moiety to the N-terminus of the GDF15 moiety. Preferably, the linker is selected from (GGGGS)ₙ (SEQ ID NO:46) and (GPPGS)ₙ (SEQ ID NO:48), wherein n is one to about 20. Preferred linkers include ((GGGGS)ₙ (SEQ ID NO: 46) and (GPPGS)ₙ (SEQ ID NO:48), wherein n is 1, 2, 3 or 4.

In more particular embodiments, the fusion polypeptide comprises HSA or a functional variant thereof, a linker, and mature human GDF15 polypeptide or a functional variant thereof and has an amino acid sequence that has at least about 90%, at least about 95%, at least about 96%, at least about 97%, at least about 98%, or at least about 99% amino acid sequence identity to any of SEQ ID NOs:20, 26, 28, 30, 32, 38, 40 and 42.

In even more particular embodiments, the fusion polypeptide has the amino acid sequence of SEQ ID NOs: 20, 26, 28, 30, 32, 38, 40 and 42.

If desired, the fusion polypeptide can contain additional amino acid sequence. For example, an affinity tag can be included to facilitate detecting and/or purifying the fusion polypeptide.

### Nucleic Acids and Host Cells

The invention also relates to nucleic acids that encode the fusion polypeptides disclosed herein, including vectors that can be used to produce the fusion polypeptides. The nucleic acids are isolated and/or recombinant. In certain embodiments, the nucleic acid encodes a fusion polypeptide in which HSA or a functional variant thereof is located N-terminally to human mature GDF15 or a functional variant thereof. If desired the nucleic acid can further encode a linker (e.g., a flexible peptide linker) that bonds the C-terminus of the HSA or a functional variant thereof to the N-terminus of human mature GDF15 or a functional variant thereof. If desired, the nucleic acid can also encode a leader, or signal, sequence to direct cellular processing and secretion of the fusion polypeptide.

In preferred embodiments, the nucleic acid encodes a fusion polypeptide in which the SA moiety is HSA or a functional variant thereof and the GDF15 moiety is the mature human GDF peptide or a functional variant thereof. When present, the optional linker is preferably a flexible peptide linker. In particular embodiments, the nucleic acid encodes a fusion polypeptide that comprises A) an SA moiety selected from the group consisting of HSA(25-609) (SEQ ID NO:45), and HSA(25-609) in which Cys34 is replaced with Ser and Asn503 is replaced with Gin; and

B) a GDF15 moiety selected from the group consisting of:
human GDF15(197-308) (SEQ ID NO:44);
human GDF15(211-308) (amino acids 211-308 of SEQ ID NO: 1);
human GDF15(197-308) (SEQ ID NO:44) in which Cys203 is replaced with Ser (C203S) and Cys210 is replaced with Ser (C210S); and
human GDF15(197-308) (SEQ ID NO:44) in which Cys273 is replaced with Ser (C273S).

If desired, the encoded fusion polypeptide can further comprise a linker that links the C-terminus of the SA moiety to the N-terminus of the GDF15 moiety. Preferably, the linker is selected from (GGGGS)ₙ and (GPPGS)ₙ (SEQ ID NO: 46) and (GPPGS)ₙ (SEQ ID NO:48), wherein n is one to about 20. Preferred linkers include ((GGGGS)ₙ (SEQ ID NO: 46) and (GPPGS)ₙ (SEQ ID NO:48), wherein n is 1, 2, 3 or 4.

In particular embodiments, the nucleic acid has a nucleotide sequence that has at least about at least about 80%, at least about 85%, at least about 90%, or at least about 95% amino acid sequence identity with any of SEQ ID NOS: 19, 25, 27, 29, 31, 37, 39 and 41, preferably when measured over the full length of SEQ ID NO: 19, 25, 27, 29, 31, 37, 39 or 41.

In more particular embodiments, the nucleic acid has the nucleotide sequence of SEQ ID NO:19, 25, 27, 29, 31, 37, 39 or 41.

For expression in host cells, the nucleic acid encoding a fusion polypeptide can be present in a suitable vector and after introduction into a suitable host, the sequence can be expressed to produce the encoded fusion polypeptide according to standard cloning and expression techniques, which are known in the art (e.g., as described in Sambrook, J., Fritsh, E. F., and Maniatis, T. Molecular Cloning: A Laboratory Manual 2^{nd}, ed., Cold Spring Harbor Laboratory, Cold Spring Harbor Laboratory Press, Cold Spring Harbor, N.Y., 1989). The invention also relates to such vectors comprising a nucleic acid sequence according to the invention.

A recombinant expression vector can be designed for expression of a GDF15 fusion polypeptide in prokaryotic (e.g., E. coli) or eukaryotic cells (e.g., insect cells, yeast cells, or mammalian cells). Representative host cells include many E. coli strains, mammalian cell lines, such as CHO, CHO-K1, and HEK293; insect cells, such as Sf9 cells; and yeast cells, such as S. cerevisiae and P. pastoris. Alternatively, the recombinant expression vector can be transcribed and translated in vitro, for example using T7 promoter regulatory sequences and T7 polymerase and an in vitro translation system. Vectors suitable for expression in host cells and cell-free in vitro systems are well known in the art. Generally such a vector contains one or more expression control elements that are operably linked to the sequence encoding the fusion polypeptide. Expression control elements include, for example, promoters, enhancers, splice sites, poly adenylation signals and the like. Usually a promoter is located upstream and operably linked to the nucleic acid sequence encoding the fusion polypeptide. The vector can comprise or be associated with any suitable promoter, enhancer, and other expression-control elements. Examples of such elements include strong expression promoters (e.g., a human CMV IE promoter/enhancer, an RSV promoter, SV40 promoter, SL3-3 promoter, MMTV promoter, or HIV LTR promoter, EFI alpha promoter, CAG promoter) and effective poly (A) termination sequences. Additional elements that can be present in a vector to facilitate cloning and propagation include, for example, an origin of replication for plasmid product in E. coli, an antibiotic resistance gene as a selectable marker, and/or a convenient cloning site (e.g., a polylinker).

In another aspect of the instant disclosure, host cells comprising the nucleic acids and vectors disclosed herein are provided. In various embodiments, the vector or nucleic acid is integrated into the host cell genome, which in other embodiments the vector or nucleic acid is extra-chromosomal. If desired the host cells can be isolated.

Recombinant cells, such as yeast, bacterial (e.g., E. coli), and mammalian cells (e.g., immortalized mammalian cells) comprising such a nucleic acid, vector, or combinations of either or both thereof are provided. In various embodiments, cells comprising a non-integrated nucleic acid, such as a plasmid, cosmid, phagemid, or linear expression element, which comprises a sequence coding for expression of a fusion polypeptide comprising the human serum albumin or the functional variant thereof and human GDF15 protein or a functional variant thereof, are provided.

A vector comprising a nucleic acid sequence encoding a GDF15 fusion polypeptide provided herein can be introduced into a host cell using any suitable method, such as by transformation, transfection or transduction. Suitable methods are well known in the art. In one example, a nucleic acid encoding a fusion polypeptide comprising the human serum albumin or the functional variant thereof and human GDF15 protein or the functional variant thereof can be positioned in and/or delivered to a host cell or host animal via a viral vector. Any suitable viral vector can be used in this capacity.

The invention also provides a method for producing a fusion polypeptide as described herein, comprising maintaining a recombinant host cell comprising a recombinant nucleic acid of the invention under conditions suitable for expression of the recombinant nucleic acid, whereby the recombinant nucleic acid is expressed and a fusion polypeptide is produced. In some embodiments, the method further comprises isolating the fusion polypeptide.

### Therapeutic Methods and Pharmaceutical Compositions

The invention also relates to methods for decreasing appetite, decreasing body weight and treating metabolic diseases in a subject in need thereof, said method comprising administering to the subject in need thereof an effective amount of a GDF15 fusion polypeptide (usually in the form of a pharmaceutical composition) as described herein. The invention also relates to methods for treating type 2 diabetes mellitus, obesity, pancreatitis, dyslipidemia, nonalcoholic steatohepatitis, insulin resistance, hyperinsulinemia, glucose intolerance, hyperglycemia, metabolic syndrome, hypertension, cardiovascular disease, atherosclerosis, peripheral arterial disease, stroke, heart failure, coronary heart disease, diabetic complications (including but not limited to chronic kidney disease), neuropathy, gastroparesis and other metabolic disorders or body weight disorders in a subject in need thereof, said method comprising administering to the subject in need thereof an effective amount of a GDF15 fusion polypeptide (usually in the form of a pharmaceutical composition) as described herein. In particular aspects, the invention relates to a methods for treating genetic obesity in a subject in need thereof, such as a subject with Prader-Willi syndrome, leptin mutations and/or melanocortin 4 receptor mutations, said method comprising administering to the subject in need thereof an effective amount of a GDF15 fusion polypeptide (usually in the form of a pharmaceutical composition) as described herein.

Subjects who are overweight or obese are at increased risk for a variety of metabolic diseases and serious health problems. These often appear first as part of the metabolic syndrome, which is characterized by elevated blood pressure, high blood sugar, excess body fat around the abdomen and abnormal blood cholesterol levels. Serious health problems can then develop, such as, type II diabetes, hypertension, coronary heart disease, stroke, cancer, osteoarthritis, sleep apnea, dyslipidemia, elevated insulin (insulin resistance), and hypoventilation syndrome. Type II diabetes (T2DM) can also give rise to several other serious health problems, such as diabetic neuropathy, diabetic nephropathy, and diabetic retinopathy. Subjects in need of therapy using a fusion polypeptide as described herein are generally overweight or obese. Generally, an adult human is considered to be overweight if he has a body mass index (BMI) between 25 and 29.9, and is considered to be obese if he has a BMI of 30 or higher. Subjects who are at increased risk of developing a metabolic diseases are also candidates for therapy using a fusion polypeptide as described herein. For example, subjects with pre-diabetes or an elevated fasting blood glucose level of 100 to 125 mg/dL are candidates for therapy, as are subjects with type II diabetes (those with fasting blood glucose levels of 126 mg/dL or higher).

Current therapeutic options comprise lifestyle modification (diet and exercise), bariatric surgery or drug therapy. Diet and exercise improvements rarely result in durable weight loss due to physiological counter-regulatory systems. Bariatric surgery carries considerable risk and is not sufficiently scalable to address the current obesity epidemic. Pharmacotherapy is limited to only a few approved agents with limited efficacy. These include phentermine (approved only for short-term use), the fat absorption inhibitor orlistat, lorcaserin (Belviq, a serotonin 5HT2c receptor agonist), and the fixed-dose combination of topiramate and phentermine (Qsymia). Qsymia is the most efficacious, reporting ˜10% placebo-adjusted weight loss over 2 years, but has several safety concerns including birth defects and elevated blood pressure.

An effective amount of the fusion polypeptide, usually in the form of a pharmaceutical composition, is administered to a subject in need thereof. The fusion polypeptide can be administered in a single dose or multiple doses, and the amount administered and dosing regimen will depend upon the particular fusion protein selected, the severity of the subject's condition and other factors. A clinician of ordinary skill can determined appropriate dosing and dosage regimen based on a number of other factors, for example, the individual's age, sensitivity, tolerance and overall well-being.

The administration can be performed by any suitable route using suitable methods, such as parenterally (e.g., intravenous, subcutaneous, intraperitoneal, intramuscular, intrathecal injections or infusion), orally, topically, intranasally or by inhalation. Parental administration is generally preferred. Subcutaneous administration is preferred.

The GDF15 fusion polypeptides of the present invention can be administered to the subject in need thereof alone or with one or more other agents. When the fusion polypeptide is administered with another agent, the agents can be administered concurrently or sequentially to provide overlap in the therapeutic effects of the agents. Examples of other agents that can be administered in combination with the fusion polypeptide include:

1. Antidiabetic agents, such as insulin, insulin derivatives and mimetics; insulin secretagogues such as the sulfonylureas (e.g., chlorpropamide, tolazamide, acetohexamide, tolbutamide, glyburide, glimepiride, glipizide); glyburide and Amaryl; insulinotropic sulfonylurea receptor ligands such as meglitinides, e.g. nateglinide and repaglinide; thiazolidinediones (e.g., rosiglitazone (AVANDIA), troglitazone (REZULIN), pioglitazone (ACTOS), balaglitazone, rivoglitazone, netoglitazone, troglitazone, englitazone, ciglitazone, adaglitazone, darglitazone that enhance insulin action (e.g., by insulin sensitization), thus promoting glucose utilization in peripheral tissues; protein tyrosine phosphatase-1B (PTP-1B) inhibitors such as PTP-112; Cholesteryl ester transfer protein (CETP) inhibitors such as torcetrapib, GSK3 (glycogen synthase kinase-3) inhibitors such as SB-517955, SB-4195052, SB-216763, NN-57-05441 and NN-57-05445; RXR ligands such as GW-0791 and AGN-194204; sodium-dependent glucose cotransporter inhibitors such as T-1095; glycogen phosphorylase A inhibitors such as BAY R3401; biguanides such as metformin and other agents that act by promoting glucose utilization, reducing hepatic glucose production and/or diminishing intestinal glucose output; alpha-glucosidase inhibitors such as acarbose and migiitoi) and other agents that slow down carbohydrate digestion and consequently absorption from the gut and reduce postprandial hyperglycemia; GLP-1 (glucagon like peptide-1), GLP-1 analogs such as Exendin-4 and GLP-1 mimetics; and DPPIV (dipeptidyl peptidase IV) inhibitors such as vildagliptin;

2. Hypolipidemic agents such as 3-hydroxy-3-methyl-glutaryl coenzyme A (HMG-CoA) reductase inhibitors, e.g. lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; bile acid sequenstrants, such as cholestyramine and colesevelam; fibrates; nicotinic acid and aspirin;

3. Anti-obesity agents such as orlistat, rimonabant, phentermine, topiramate, qunexa, and locaserin;

4. Anti-hypertensive agents, e.g. loop diuretics such as ethacrynic acid, furosemide and torsemide; angiotensin converting enzyme (ACE) inhibitors such as benazepril, captopril, enalapril, fosinopril, lisinopril, moexipril, perinodopril, quinapril, ramipril and trandolapril; inhibitors of the Na-K-ATPase membrane pump such as digoxin; neutralendopeptidase (NEP) inhibitors; ACE/NEP inhibitors such as omapatrilat, sampatrilat and fasidotril; angiotensin II antagonists such as candesartan, eprosartan, irbesartan, losartan, telmisartan and valsartan, in particular valsartan; renin inhibitors such as ditekiren, zankiren, terlakiren, aliskiren, RO 66-1132 and RO-66-1168; f-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; and aldosterone synthase inhibitors;

5. Agonists of peroxisome proliferator-activator receptors, such as fenofibrate, pioglitazone, rosiglitazone, tesaglitazar, BMS-298585, L-796449, the compounds specifically described in the patent application WO 2004/103995 i.e. compounds of examples 1 to 35 or compounds specifically listed in claim 21, or the compounds specifically described in the patent application WO 03/043985 i.e. compounds of examples 1 to 7 or compounds specifically listed in claim 19 and especially (R)-1-{4-[5-methyl-2-(4-trifluoromethyl-phenyl)-oxazol-4-ylmethoxy]-benzenesulfonyl}-2,3-dihydro-1H-indole-2-carboxylic or a salt thereof; and

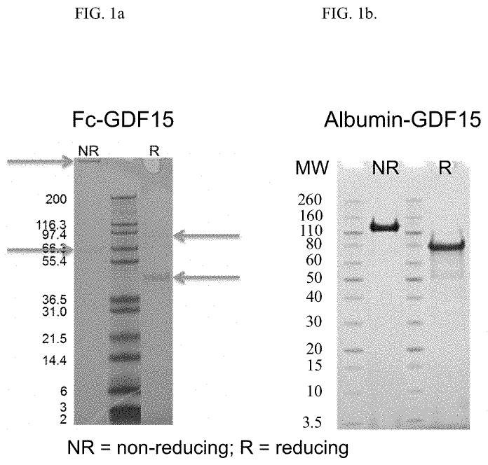

6. The specific anti-diabetic compounds described in Expert Opin Investig Drugs 2003, 12(4): 623-633, FIGS. 1 to 7.

The invention also relates to pharmaceutical compositions comprising a GDF15 fusion polypeptide as described herein (e.g., comprising a fusion polypeptide comprising human serum albumin or a functional variant thereof and human GDF15 protein or a functional variant thereof). Such pharmaceutical compositions can comprise a therapeutically effective amount of the fusion polypeptide and a pharmaceutically or physiologically acceptable carrier. The carrier is generally selected to be suitable for the intended mode of administration and can include agents for modifying, maintaining, or preserving, for example, the pH, osmolarity, viscosity, clarity, color, isotonicity, odor, sterility, stability, rate of dissolution or release, adsorption, or penetration of the composition. Typically, these carriers include aqueous or alcoholic/aqueous solutions, emulsions or suspensions, including saline and/or buffered media.

Suitable agents for inclusion in the pharmaceutical compositions include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as free serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as Polysorbate 20 or Polysorbate 80; Triton; tromethamine; lecithin; cholesterol or tyloxapal), stability enhancing agents (such as sucrose or sorbitol), tonicity enhancing agents (such as alkali metal halides, such as sodium or potassium chloride, or mannitol sorbitol), delivery vehicles, diluents, excipients and/or pharmaceutical adjuvants

Parenteral vehicles include sodium chloride solution, Ringer's dextrose, dextrose and sodium chloride and lactated Ringer's. Suitable physiologically-acceptable thickeners such as carboxymethylcellulose, polyvinylpyrrolidone, gelatin and alginates may be included. Intravenous vehicles include fluid and nutrient replenishers and electrolyte replenishers, such as those based on Ringer's dextrose. In some cases it will be preferable to include agents to adjust tonicity of the composition, for example, sugars, polyalcohols such as mannitol, sorbitol, or sodium chloride in a pharmaceutical composition. For example, in many cases it is desirable that the composition is substantially isotonic. Preservatives and other additives, such as antimicrobials, antioxidants, chelating agents and inert gases, may also be present. The precise formulation will depend on the route of administration. Additional relevant principle, methods and components for pharmaceutical formulations are well known. (See, e.g., Allen, Loyd V. Ed, (2012) Remington's Pharmaceutical Sciences, 22th Edition)

When parenteral administration is contemplated, the pharmaceutical compositions are usually in the form of a sterile, pyrogen-free, parenterally acceptable composition. A particularly suitable vehicle for parenteral injection is a sterile, isotonic solution, properly preserved. The pharmaceutical composition can be in the form of a lyophilizate, such as a lyophilized cake.

In certain embodiments, the pharmaceutical composition is for subcutaneous administration. Suitable formulation components and methods for subcutaneous administration of polypeptide therapeutics (e.g., antibodies, fusion proteins and the like) are known in the art. See, e.g., Published United States Patent Application No 2011/0044977 and U.S. Pat. Nos. 8,465,739 and 8,476,239. Typically, the pharmaceutical compositions for subcutaneous administration contain suitable stabilizers (e.g, amino acids, such as methionine, and or saccharides such as sucrose), buffering agents and tonicifying agents.

### Definitions

The term “amino acid mimetic,” as used herein, refers to chemical compounds that have a structure that is different from the general chemical structure of an amino acid, but functions in a manner similar to a naturally occurring amino acid.

“Atherosclerosis” is a vascular disease characterized by irregularly distributed lipid deposits in the intima of large and medium-sized arteries, sometimes causing narrowing of arterial lumens and proceeding eventually to fibrosis and calcification. Lesions are usually focal and progress slowly and intermittently. Limitation of blood flow accounts for most clinical manifestations, which vary with the distribution and severity of lesions.

As used herein, the phrase “body weight disorder” refers to conditions associated with excessive body weight and/or enhanced appetite. Various parameters are used to determine whether a subject is overweight compared to a reference healthy individual, including the subject's age, height, sex and health status. For example, a subject may be considered overweight or obese by assessment of the subject's Body Mass Index (BMI), which is calculated by dividing a subject's weight in kilograms by the subject's height in meters squared. An adult having a BMI in the range of −18.5 to −24.9 kg/m is considered to have a normal weight; an adult having a BMI between −25 and −29.9 kg/m may be considered overweight (pre-obese); an adult having a BMI of −30 kg/m or higher may be considered obese. Enhanced appetite frequently contributes to excessive body weight. There are several conditions associated with enhanced appetite, including, for example, night eating syndrome, which is characterized by morning anorexia and evening polyphagia often associated with insomnia, but which may be related to injury to the hypothalamus.

“Cardiovascular diseases” are diseases related to the heart or blood vessels.

“Conservative” amino acid replacements or substitutions refer to replacing one amino acid with another that has a side chain with similar size, shape and/or chemical characteristics. Examples of conservative amino acid replacements include replacing one amino acid with another amino acid within the following groups: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

“Coronary heart disease”, also called coronary artery disease, is a narrowing of the small blood vessels that supply blood and oxygen to the heart.

“Diabetic complications” are problems caused by high blood glucose levels, with other body functions such as kidneys (nephropathies), nerves (neuropathies), feet (foot ulcers and poor circulation) and eyes (e.g. retinopathies). Diabetes also increases the risk for heart disease and bone and joint disorders. Other long-term complications of diabetes include skin problems, digestive problems, sexual dysfunction and problems with teeth and gums.

“Dyslipidemia” is a disorder of lipoprotein metabolism, including lipoprotein overproduction or deficiency. Dyslipidemias may be manifested by elevation of the total cholesterol, low-density lipoprotein (LDL) cholesterol and triglyceride concentrations, and a decrease in high-density lipoprotein (HDL) cholesterol concentration in the blood.

The term “effective amount” refers to is an amount sufficient to achieve the desired therapeutic effect, under the conditions of administration, such as an amount sufficient to decrease appetite, cause weight loss, decrease fat mass, decrease fasting glucose levels, insulin release, and/or food intake. For example, a “therapeutically-effective amount” administered to a patient exhibiting, suffering, or prone to suffer from metabolic disorders (such as T2DM, obesity, or metabolic syndrome), is such an amount which causes an improvement in the pathological symptoms, disease progression, physiological conditions associated with or induces resistance to succumbing to the afore mentioned disorders.

“Functional variant” and “biologically active variant” refers to a polypeptide that contains an amino acid sequence that differs from a reference polypeptide (e.g., HSA, human wild type mature GDF15 peptide) but retains desired functional activity of the reference polypeptide. The amino acid sequence of a functional variant can include one or more amino acid replacements, additions or omissions relative to the reference polypeptide, and include fragments of the reference polypeptide that retain the desired activity. For example, a functional variant of SA (e.g., HSA) prolongs the serum half-life of the fusion polypeptides described herein in comparison to the half-life of GDF15, while retaining the reference GDF15 (e.g., human GDF15) polypeptide's activity (e.g., weight loss, appetite suppressing, insulin release, insulin sensitivity, and/or fat mass reduction) activity. Polypeptide variants possessing a somewhat decreased level of activity relative to their wild-type versions can nonetheless be considered to be functional or biologically active polypeptide variants, although ideally a biologically active polypeptide possesses similar or enhanced biological properties relative to its wild-type protein counterpart (a protein that contains the reference amino acid sequence).

The phrase “glucose tolerance”, as used herein, refers to the ability of a subject to control the level of plasma glucose and/or plasma insulin when glucose intake fluctuates. For example, glucose tolerance encompasses the subject's ability to reduce, within about 120 minutes, the level of plasma glucose back to a level determined before the intake of glucose.

“Glucose intolerance, or ‘Impaired Glucose Tolerance (IGT) is a pre-diabetic state of dysglycemia that is associated with increased risk of cardiovascular pathology. The pre-diabetic condition prevents a subject from moving glucose into cells efficiently and utilizing it as an efficient fuel source, leading to elevated glucose levels in blood and some degree of insulin resistance.

The phrase “glucose metabolism disorder” encompasses any disorder characterized by a clinical symptom or a combination of clinical symptoms that is associated with an elevated level of glucose and/or an elevated level of insulin in a subject relative to a healthy individual. Elevated levels of glucose and/or insulin may be manifested in the following diseases, disorders and conditions: hyperglycemia, type II diabetes, gestational diabetes, type I diabetes, insulin resistance, impaired glucose tolerance, hyperinsulinemia, impaired glucose metabolism, pre-diabetes, metabolic disorders (such as metabolic disease or disorder, which is also referred to as syndrome X), and obesity, among others. The GDF15 conjugates of the present disclosure, and compositions thereof, can be used, for example, to achieve and/or maintain glucose homeostasis, e.g., to reduce glucose level in the bloodstream and/or to reduce insulin level to a range found in a healthy subject.

“Hyperglycemia” refers to a condition in which an elevated amount of glucose circulates in the blood plasma of a subject relative to a healthy individual. Hyperglycemia can be diagnosed using methods known in the art, including measurement of fasting blood glucose levels as described herein.

“Hyperinsulinemia” refers to a condition in which there are elevated levels of circulating insulin when, concomitantly, blood glucose levels are either elevated or normal. Hyperinsulinemia can be caused by insulin resistance which is associated with dyslipidemia such as high triglycerides, high cholesterol, high low-density lipoprotein (LDL) and low high-density lipoprotein (HDL); high uric acids levels; polycystic ovary syndrome; type II diabetes and obesity. Hyperinsulinemia can be diagnosed as having a plasma insulin level higher than about 2 pU/mL.

“Hypoglycemia”, also called low blood sugar, occurs when blood glucose level drops too low to provide enough energy for the body's activities.

“Identity” means, in relation to nucleotide or amino acid sequence of a nucleic acid or polypeptide molecule, the overall relatedness between two such molecules. Calculation of the percent sequence identity (nucleotide or amino acid sequence identity) of two sequences, for example, can be performed by aligning the two sequences for optimal comparison purposes (e.g., gaps can be introduced in one or both of a first and a second nucleic acid or amino acid sequence for optimal alignment). The nucleotides or amino acids at corresponding positions are then compared. When a position in the first sequence is occupied by the same nucleotide or amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two sequences can be determined using methods such as those described by the National Center for Biotechnology Information (http://www.ncbi.nlm.nih.gov/). For example, the percent identity between two sequences can be determined using Clustal 2.0 multiple sequence alignment program and default parameters. Larkin M A et al. (2007) “Clustal W and Clustal X version 2.0.” Bioinformatics 23(21): 2947-2948.

“Insulin resistance” is defined as a state in which a normal amount of insulin produces a subnormal biologic response.

The term “metabolic diseases,” and terms similarly used herein, includes but is not limited to obesity, T2DM, pancreatitis, dyslipidemia, nonalcoholic steatohepatitis (NASH), insulin resistance, hyperinsulinemia, glucose intolerance, hyperglycemia, metabolic syndrome, hypertension, cardiovascular disease, atherosclerosis, peripheral arterial disease, stroke, heart failure, coronary heart disease, diabetic complications (including but not limited to chronic kidney disease), neuropathy, gastroparesis and other metabolic disorders.

The term “metabolic disease or disorder” refers to an associated cluster of traits that includes, but is not limited to, hyperinsulinemia, abnormal glucose tolerance, obesity, redistribution of fat to the abdominal or upper body compartment, hypertension, dyslipidemia characterized by high triglycerides, low high density lipoprotein (HDL)-cholesterol, and high small dense low density lipoprotein (LDL) particles. Subjects having metabolic disease or disorder are at risk for development of Type 2 diabetes and, for example, atherosclerosis.

“Metabolic syndrome” can be defined as a cluster of risk factors that raises the risk for heart disease and other diseases like diabetes and stroke. These risk factors include: abdominal fat—in most men a waist:hip ratio>0.9 or BMI>30 kg/m2; high blood sugar—at least 110 milligrams per deciliter (mg/dl) after fasting; high triglycerides—at least 150 mg/dL in the bloodstream; low HDL—less than 40 mg/dl; and, blood pressure of 130/85 mmHg or higher (World Health Organization).

The term “moiety”, as used herein, refers to a portion of a fusion polypeptide described herein. The fusion polypeptides include a GDF15 moiety, which contains an amino acid sequence derived from GDF15, and an SA moiety, which contain an amino acid sequence derived from SA. The fusion protein optionally contains a linker moiety, which links the DGF15 moiety and the SA moiety, in the fusion polypeptide. Without wishing to be bound by any particular theory, it is believed that the GDF15 moiety confers biological function of decreasing appetite, promoting weight loss and treating obesity and other metabolic diseases, while the SA moiety prolongs the serum half-life, improves expression and stability of the fusion polypeptides described herein.

The term “naturally occurring” when used in connection with biological materials such as nucleic acid molecules, polypeptides, host cells, and the like, refers to materials that are found in nature and are not manipulated by man. Similarly, “non-naturally occurring” as used herein refers to a material that is not found in nature or that has been structurally modified or synthesized by man. When used in connection with nucleotides, the term “naturally occurring” refers to the bases adenine (A), cytosine (C), guanine (G), thymine (T), and uracil (U). When used in connection with amino acids, the term “naturally occurring” refers to the 20 conventional amino acids (i.e., alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), and tyrosine (Y)), as well as selenocysteine, pyrrolysine (PYL), and pyrroline-carboxy-lysine (PCL).

“Nonalcoholic steatohepatitis (NASH)” is a liver disease, not associated with alcohol consumption, characterized by fatty change of hepatocytes, accompanied by intralobular inflammation and fibrosis.

“Obesity,” in terms of the human subject, can be defined as an adult with a Body Mass Index (BMI) of 30 or greater (Centers for Disease Control and Prevention).

“Pancreatitis” is inflammation of the pancreas.

As used herein, the terms “variant,” “mutant,” as well as any like terms, when used in reference to GDF15 or SA or specific versions thereof (e.g., “GDF15 protein variant,” “human GDF15 variant,” etc.) define protein or polypeptide sequences that comprise modifications, truncations, or other variants of naturally occurring (i.e., wild-type) protein or polypeptide counterparts or corresponding native sequences. “Variant GDF15” or “GDF15 mutant,” for instance, is described relative to the wild-type (i.e., naturally occurring) GDF15 protein as described herein and known in the literature.

A “subject” is an individual to whom a fusion polypeptide is administered. The subject is preferably a human, but “subject” includes pet and livestock animals, such as cows, sheep, goats, horses, dogs, cats, rabbits, guinea pigs, rats, mice or other bovine, ovine, equine, canine, feline, rodent or murine species, poultry and fish.

“Type 2 diabetes mellitus” or “T2DM” is a condition characterized by excess glucose production and circulating glucose levels remain excessively high as a result of inadequate glucose clearance and the inability of the pancreas to produce enough insulin.

### Examples

The following examples, including the experiments conducted and results achieved, are provided for illustrative purposes only and are not to be construed as limiting the present invention.

### I. Expression and Purification of Fusion Polypeptides

A. Mammalian Cell Expression and Purification of Albumin-Human GDF15 Fusions

Constructs of albumin-human GDF15 fusion proteins were expressed in transiently transfected HEK293F cells. Briefly, a liter of HEK293F cells 1 mg of DNA and 3 mg of linear 25 kDa polyethylenimine were mixed in 100 mL of medium, incubated at room temperature for 10 minutes, and then added to the cells. The cells were incubated for 5 days post transfection at 37° C. at 125 rpm (50 mm throw) at 8% CO₂ at 80% humidity. The cells were removed by centrifugation for 20 minutes at 6,000×g at 4° C. The supernatant was filtered through a 0.8/0.2 μm membrane and buffer exchanged into 100 mM TRIS pH 8.0 by TFF. The GDF15 constructs were captured on a Q Sepharose anion exchange column and eluted in a 10 column volume gradient from 0-400 mM NaCl in 100 mM TRIS pH 8.0. The fractions containing GDF15 were further purified by size exclusion chromatography in 1×DPBS, 1.47 mM KH₂PO₄, 8.06 mM Na₂HPO₄-7H₂O, 137.9 mM NaCl, 2.67 mM KCl. The fractions containing GDF15 were flask frozen in liquid nitrogen and stored at −80° C.

Mammalian Cell Expression and Purification of his-Human GDF15 Fusion Proteins

Constructs of His-human GDF15 fusion proteins were expressed in transiently transfected HEK293F cells. Briefly, per 2.5 liters of HEK293F cells 2.5 mg of DNA and 7.5 mg of linear 25 kDa polyethylenimine were mixed in 250 mL of medium, incubated at room temperature for 10 minutes, and then added to the cells. The cells were incubated for 4 days post transfection at 37° C. at 125 rpm (50 mm throw) at 8% CO₂ at 80% humidity. The cells were removed by centrifugation for 20 minutes at 6,000×g at 4° C. The supernatant was filtered through a 0.8/0.2 μm membrane. 1 M citric acid pH 3 was added to the filtered supernatant to a final concentration of 135 mM, solid sodium chloride was added to a final concentration of 2 M, and the supernatant was filtered through a 0.22 μm membrane. 5 mL of phenyl sepharose resin were equilibrated in 100 mM citric acid, 2 M NaCl, pH 3 and added to the supernatant. The resin was incubated with the supernatant for 2 hours at room temperature and packed into a 5 cm gravity column. The resin was washed with 20 mL of 100 mM citric acid, 2 M NaCl, pH 3; 20 mL of 100 mM citric acid, 1.5 M NaCl, pH 3; 100 mM citric acid, 1 M NaCl, pH 3; 100 mM citric acid, 0.5 M NaCl, pH 3; 100 mM citric acid, pH 3; 100 mM citric acid, 20% ethanol, pH 3; and 100 mM citric acid, 50% ethanol, pH 3. The washes containing no NaCl were pooled. 2 M TRIS base added to the phenyl sepharose pool to a final concentration of 180 mM yielding a final pH of 7.5. 5 M NaCl was added to a final concentration of 150 mM. 160 μL of Ni Sepharose HP resin were equilibrated in PBS, added to the phenyl sepharose pool, and incubated for 1 hour at room temperature. The resin was packed into a 1 cm gravity column and washed with 20 mL of PBS followed by 1 mL of PBS+100 mM imidazole. The bound protein was eluted in 1 mL of PBS+500 mM imidazole. The fractions containing GDF15 were flash frozen in liquid nitrogen and stored at −80° C.

B. Yeast Expression and Purification

Constructs of human GDF15 were expressed in Pichia pastoris utilizing methanol induction. Plasmid DNA was linearized with SacI for use in transformation. The linearized DNA was transformed into Pichia pastoris strain SMD1168 and expressed in BMMY medium at pH 6 with 1% (v/v) methanol at 30° C. at 200 rpm (1 inch throw) for 4 days. Methanol was added to a final concentration of 1% (v/v) each day during expression. The cells were removed by centrifugation for 20 minutes at 5,000×g at 4° C. and the supernatant was filtered through a 0.22 μm membrane. An equal volume of 1 M citric acid, 3 M NaCl pH 2.75 was added to the filtered supernatant. Phenyl Sepharose 6 was added to the supernatant and the GDF15 was bound by incubation for 1 hour at room temperature while stirring. The resin was packed into a gravity column and the flow-through was removed. The resin was washed with 25 column volumes of 0.5 M citric acid, 1.5 M NaCl pH 3, 5 column volumes of 100 mM citric acid pH 3, and 5 column volumes of 100 mM citric acid, 20% ethanol pH 3. The bound protein was eluted in 5×1 column volume of 100 mM citric acid, 50% ethanol, pH 3. The elution fractions containing GDF15 were combined, diluted 1:10 into 25 mM bis-TRIS pH 5, and filtered through a 0.22 μm membrane. SP Sepharose cation exchange resin was added to the GDF15 and incubated for 1 hour at room temperature. The resin was packed into a gravity column and the flow-through was removed. The column was washed with 50 column volumes of 25 mM bis-TRIS pH 5 and eluted in 10 column volumes of 50 mM sodium phosphate, 150 mM NaCl pH 6.2.

C. E. coli Expression

E coli produced GDF15 was fused to a modified autoprotease P20 from Classical swine fever virus and expressed in inclusion bodies. E. coli transformed with GDF15 plasmid DNA were grown for 60 hours at 30° C. in ZYP-5052 auto induction medium (Studier F. W., Protein Expression and Purification 41 (2005) 207-234). The cell pellet was harvested by centrifugation for 30 minutes at 5,000×g at 18° C. Per liter of culture, the pellet was resuspended in 250 mL of 100 mM TRIS pH 8, 150 mM NaCl, 3 mM EDTA, 0.01% (v/v) Triton X-100, 1 mg/mL lysozyme and incubated for 20 minutes at room temperature, rotating. 250 mL of 100 mM TRIS pH 8, 150 mM NaCl, 20 mM CaCl₂), 20 mM MgCl₂, 0.25 mg/mL DNase I was added followed by an incubation for 20 minutes at room temperature, stirring. The pellet was centrifuged for 15 minutes at 5,000×g at 18° C. and the supernatant was discarded. The pellet was resuspended in 500 mL of 2% (v/v) Triton X-100 and incubated for 20 minutes at room temperature, rotating. The pellet was centrifuged for 15 minutes at 5,000×g at 18° C. and the supernatant was discarded. The pellet was resuspended in 500 mL of 500 mM NaCl and incubated for 20 minutes at room temperature, rotating. The pellet was centrifuged for 20 minutes at 5,000×g at 18° C. and the supernatant was discarded. The pellet was resuspended in 500 mL of 100 mM TRIS pH 8, 150 mM NaCl, 20 mM CaCl₂), 20 mM MgCl₂, 0.25 mg/mL DNase I and incubated for 20 minutes at room temperature, rotating. The pellet was centrifuged for 20 minutes at 5,000×g at 18° C. and the supernatant was discarded. The pellet was resuspended in 500 mL of 80% (v/v) ethanol and incubated for 20 minutes at room temperature, rotating. The pellet was centrifuged for 20 minutes at 5,000×g at 18° C. and the supernatant was discarded. The pellet was resuspended in 500 mL 100 mM TRIS pH 8, 500 mM NaCl, 8 M urea and incubated for 1 hour at room temperature, rotating. 10 mL of Ni Sepharose High Performance resin were added and incubated at room temperature for 1 hour, rotating. The resin was packed into a gravity column and the flow-through was discarded. The resin was washed with 25 column volumes of 100 mM TRIS pH 8, 500 mM NaCl, 8 M urea the 25 column volumes of 100 mM TRIS pH 8, 1 M NaCl, 2 M urea. The bound protein was eluted in 2×5 column volumes of 100 mM TRIS pH 8, 1 M NaCl, 2 M urea, 0.5 M imidazole. The eluted protein was diluted 1:10 into 1 M TRIS-base, 1 M NaCl, 0.2 M histidine, 10 mM TCEP, pH 8.5. The sample was stirred briefly to mix and incubated overnight at room temperature with no agitation. The sample was loaded over a 6 gram HLB cartridge, washed in 100 mL of 0.1% (v/v) formic acid in water, and eluted in 50 mL of 0.1% (v/v) formic acid in isopropanol. The HLB elution was diluted 1:20 into 1 liter of 50 mM HEPES, 500 mM NaCl, 2 mM TCEP, 8 M urea, pH 7.6. 10 mL of Ni Sepharose High Performance resin were added and incubated at room temperature for 1 hour, stirring. The resin was packed into a gravity column and the flow-though was saved. The Ni flow-though was loaded over a 6 gram HLB cartridge, washed in 100 mL of 0.1% (v/v) formic acid in water, and eluted in 50 mL of 0.1% (v/v) formic acid in isopropanol. The second HLB elution was diluted 1:20 into 1 liter of 100 mM TRIS pH 8, 0.5 M urea, 2 mM oxidized glutathione, 2 mM reduced glutathione. The sample was stirred briefly to mix and incubated overnight at room temperature with no agitation. 100 mL of 5 M NaCl were added to make a final concentration of 500 mM and the sample was loaded over a 6 gram HLB cartridge. The cartridge was washed with 100 mL of 0.1% (v/v) formic acid in water and eluted in 25 mL of 0.1% (v/v) formic acid in ethanol. The HLB elution was diluted 1:4 by the addition of 75 mL of 50 mM bis-TRIS pH 4.8 and 1 mL of SP Sepharose resin was added. The resin was incubated with the GDF15 for 1 hour at room temperature and the packed into a gravity column. The resin was washed with 1 mL of 50 mM bis-TRIS pH 4.8 and eluted in 3×1 mL of PBS pH 6.4. Fractions 1 and 2 were combined, flash frozen in liquid nitrogen, and stored at −80° C.

### II. Animal Studies

Animal Studies: All animal studies described in this document were approved by the Novartis Institutes for Biomedical Research Animal Care and Use Committee in accordance with local and federal regulations and guidelines. Male mice (C57BL/6NTac) fed either a standard laboratory chow diet or a 60% fat diet (Research Diets D12492i) from 6-weeks of age onward were purchased from Taconic. Upon arrival, mice were housed one animal per cage typically under a 12 h:12 h reverse light-dark cycle. Animals all received a minimum of 1 week acclimation prior to any use. Mice were typically studied between 3-5 months of age. Prior to being studied, mice were randomized (typically 1-day prior to the experimental period) based on body weight such that each group had a similar average body weight.

Hydrodynamic DNA injections: On the day of study, mice were placed in fresh cages, and the old food removed. Each study animal (diet-induced obese male mice) received a single hydrodynamic injection of plasmid DNA via tail vein. DNA (typically 3 micrograms/mouse) was diluted in sterile saline at a volume ˜6.5% of the animal's body weight and rapidly injected within ˜5-10 seconds. Immediately after injection, pre-weighed fresh high-fat diet diet was added to each cage at the end of the procedures above. Food intake and body weight were measured at the indicated time points.

Recombinant GDF15 analogs: On the day of study, mice were placed in fresh cages, and the old food removed. Approximately 1 h later and just prior to the dark cycle, mice received a subcutaneous dose of either vehicle (1×PBS) or a GDF15 analog at the indicated times. After all injections are completed, the mice were reweighed and a defined amount of food returned (˜50 g per mouse of standard chow or high-fat diet). Food intake and body weight were measured over the course of the study at the times indicated.

Plasma GDF15 exposure: In surrogate animals treated as described above, plasma was collected into EDTA coated tubes at the indicated times, and human GDF15 levels were measured by ELISA as per the manufacturer's instructions (R&D Systems Quantikine Human GDF15 Immunoassay; DGD 150). This assay does not recognize endogenous mouse GDF15.

Body composition: In some animals, body composition was assessed by NMR (Bruker MiniSpec Model LF90ii) as per the manufacturer's instructions. The mass of fat tissue, lean tissue and free fluid was calculated using MiniSpec software V.2.59.rev.6.

### Results

All mammalian cell expressed constructs were secreted using the mouse Igκ chain V-III region MOPC 63 signal peptide with the exception of the mouse albumin domain 1 fusion and the non 3x4GS linkers which were secreted using the human CD8A signal peptide. Yeast expressed constructs were secreted using a modified mating factor alpha-1 signal peptide.

GDF15 can cause or promote weight loss agent in mice. However, characteristics of GDF15 make the naturally occurring peptide unsuitable for use as a therapeutic in humans, such as the short lived plasma half-life (˜1 h) of the wild-type human peptide and poor expression levels in mammalian cells (Fairlie W D, et. al. Gene (2000) 254:67-76). To help understand whether GDF15 can be modified to improve its properties, e.g., extend its plasma half-life, the inventors solved the crystal structure of the protein. The GDF15 crystal structure revealed a unique disulfide pattern for GDF15 compared to other members of the TGFbeta superfamily that contain the 9 conserved cysteine residues, such as TGFB1-3 and inhibin beta (Galat A Cell. Mol. Life Sci. (2011) 68:3437-3451). To test the functional importance of these disulfide bonds, mammalian expression vectors were constructed that encoded proteins where each of the conserved cysteine residues that make up the disulfide bonds were individually mutated to serine residues. The expression constructs were delivered by hydrodynamic DNA injection to diet-induced obese mice as described in the Material and Methods section. Mice injected with the expression vector encoding naturally occurring GDF15 ate 31.1% less food and were 31.3% lighter 3 weeks post treatment compared to mice injected with the empty vector. Mice receiving the expression vector encoding mutations at C203S, C210S, or C273S ate 27.9, 28.0, and 33.9% less food and weighed 25.5, 20.4, and 30.3% less, respectively, than the control mice receiving the empty vector. Food intake and body weight were similar among empty vector treated mice and mice treated with an expression vector encoding C211S, C240S, C244S, C274S, C305S, or C307S. These data demonstrate that the first disulfide bond between C203 and C210 is not required for efficacy and suggest the amino-terminus of mature GDF15 can be manipulated. Interestingly, C273, which forms the interchain disulfide bond, is also not required for efficacy of GDF15.

The structural data combined with the functional data from the cysteine mutagenesis studies suggested that the amino terminus of GDF15 and potentially the carboxy terminus could be modified to extend the half-life of GDF15. To test this, mammalian expression vectors were constructed that encoded N-terminal Fc-GDF15 and C-terminal fusion proteins as well as mature GDF15 protein. Mice receiving a single hydrodynamic injection of an expression vector encoding mature GDF15 consistently ate approximately 25% less food than mice receiving a hydrodynamic injection of empty vector (Table 1a). By the end of 4 weeks these mice weighed 28.9% less than the control mice (Table 1b). Mice injected with a vector encoding an N-terminal Fc-GDF15 fusion protein ate about 25% less food over the first two weeks than the empty vector treated mice; however, by week 3 Fc-GDF15 treated mice were eating similar amounts of food as controls. Body weights of Fc-GDF15 treated mice also initially decreased but then started to rebound such that by 4 weeks post injection, the Fc-GDF15 mice only weighed 9.8 percent less than empty vector treated mice. In contrast, mice injected with a vector encoding a C-terminal GDF15-Fc fusion protein consumed similar levels of food and gained weight exactly like empty vector treated mice throughout the duration of the experiment. High plasma GDF15 levels were detected at 1 and 3 weeks post injection for the mature GDF15 treated group (2.6 and 1.8 nM, respectively). Plasma GDF15 levels were 2.8 nM one week post dose but were undetectable 3 weeks post injection of the vector encoding Fc-GDF15. No GDF15 was detected at any time in mice treated with the GDF15-Fc expression vector. In summary, these data indicate that the C-terminal fusion of GDF15 was inactive, while N-terminal fusion of GDF15 was active. However, the loss of expression of GDF15 in the Fc-GDF15 fusion group suggests that Fc fusions to GDF15 may not be suitable therapeutics.

TABLE 1a
Weekly Food Consumption (grams)

| | Empty Vector | Mature GDF15 | | Fc-GDF15 | | GDF15-Fc | |
|---|---|---|---|---|---|---|---|
| Week 1 | 15.1 ± 0.62 | 11.6 ± 0.34 | (−22.3) | 11.7 ± 0.52 | (−22.9) | 15.7 ± 0.69 | (3.8) |
| Week 2 | 17.4 ± 0.73 | 13.1 ± 0.47 | (−24.7) | 13.1 ± 2.64 | (−24.8) | 17.5 ± 0.72 | (0.2) |
| Week 3 | 18.0 ± 0.56 | 13.7 ± 0.51 | (−24.1) | 16.8 ± 0.49 | (−6.4) | 18.6 ± 0.54 | (3.4) |
| Week 4 | 18.4 ± 0.6 | 14.1 ± 0.62 | (−23.4) | 17.6 ± 0.18 | (−4.3) | 18.1 ± 0.52 | (−1.5) |

Mean ± SEM (Percent Change Relative to Empty Vector)

TABLE 1b
Body Weight (grams)

| | Empty Vector | Mature GDF15 | Fc-GDF15 | GDF15-Fc |
|---|---|---|---|---|
| Baseline | 31.1 ± 1.1 | 31.7 ± 0.8 | 31.0 ± 0.7 | 31.6 ± 0.8 |
| Week 1 | 30.7 ± 1.1 | 28.9 ± 0.7 | 28.3 ± 0.8 | 31.7 ± 1.1 |
| Week 2 | 32.5 ± 1.5 | 27.7 ± 0.5 | 29.4 ± 0.6 | 33.3 ± 1.1 |
| Week 3 | 34.2 ± 1.7 | 26.6 ± 0.5 | 30.9 ± 0.5 | 35.5 ± 1.2 |
| Week 4 | 36.7 ± 1.8 | 26.1 ± 0.6 | 33.1 ± 0.6 | 37.3 ± 1.4 |

Mean ± SEM

Based upon the opposing dimerization orientations of Fc and GDF15 and the loss of detectable plasma GDF15 in the Fc-GDF15 group, we suspected that Fc-GDF15 fusion proteins would be prone to aggregation, likely resulting in animals mounting an immune response against the Fc-GDF15 fusion protein. To determine if Fc-GDF15 fusion proteins are prone to aggregation, an Fc-GDF15 fusion protein was expressed in HEK293 cells. While the Fc-GDF15 fusion protein was expressed, a large proportion of the protein migrated close to the origin when analyzed under non-reducing conditions on a polyacrylamide gel, consistent with aggregation of the protein. (FIG. 1a) Further analysis by size exclusion chromatography confirmed the protein was aggregated.

In studies to identify GDF15 fusion proteins that were active but did not aggregate mammalian expression vectors encoding an N-terminal human serum albumin-[GGGGS]3-GDF15 (HSA-GDF15) fusion protein and a mouse serum albumin-[GGGGS]3-GDF15 (MSA-GDF15) were transfected into HEK293 cells. Unlike the Fc-GDF15 fusion protein, both HSA-GDF15 and MSA-GDF15 migrated at the expected molecular weight when analyzed under non-reducing conditions on a polyacrylamide gel and by size exclusion chromatography. (FIG. 1b) Unexpectedly, expression of both albumin-GDF15 fusion proteins in mammalian cells was also about 1000× greater than that for the mature GDF15 protein.

To determine if fusion of albumin to the N-terminus of GDF15 resulted in an active protein, lean mice were dosed with a single subcutaneous injection of vehicle or 99 micrograms (˜0.6 nmol of dimer) of MSA-GDF15 (197-308), MSA-GDF15 (197-308, C203S, C210S), MSA-GDF15 (211-308), or MSA-GDF15 (197-308, C273S). Compared to vehicle treated animals, food intake was reduced by 34, 34, 42, and 25 percent in animals receiving MSA-GDF15 (197-308), MSA-GDF15 (197-308, C203S, C210S), MSA-GDF15 (197-308, C273S), and MSA-GDF15 (211-308), respectively. These data clearly demonstrate that fusion of albumin to the N-terminus of GDF15 results in biologically active protein.

Fusion of albumin to the N-terminus of GDF15 also greatly increased the plasma half-life compared to the mature GDF15. The plasma half-life of mature GDF15 was ˜1 h while the plasma half-life of the N-terminal serum albumin-GDF15 fusion proteins was ˜50 h. Once weekly administration of MSA-GDF15 for 3 consecutive weeks greatly enhanced weight loss in obese mice compared to mature GDF15 at equivalent doses (0.6 nmol dimer/mouse, s.c.). Twenty eight days after the first dose and 2-weeks after the previous dose, MSA-GDF15 treated mice lost 12.8 percent of their starting body weight while, over the same duration, vehicle treated and GDF15 treated mice increased their starting body weight by an additional 10.9% and 5.6%, respectively. Analysis of body composition indicated that the weight loss induced by MSA-GDF15 is largely from fat mass with sparing of lean mass. On day 23 post initiation of dosing, the fat mass of MSA-GDF15 treated mice was 18.3% compared to 25.2% and 24.5% for vehicle and GDF15 treated mice, respectively. Lean mass in MSA-GDF15 treated mice was 55.6% of their body weight compared 51.5% and 52% for vehicle treated and GDF15 treated mice, respectively.

The HSA-GDF15 fusion was also biologically active. Obese mice receiving a single subcutaneous dose (3 mg/kg s.c.) of HSA-3x4GS-hGDF15(197-308) ate 31% less food over 24 h than vehicle-treated controls while MSA-GDF15 treated mice ate 27% less than vehicle controls. HSA-GDF15 fusions with different peptide linkers between albumin and GDF15 were also biologically active. Obese mice were treated with a single subcutaneous dose (3 mg/kg s.c.) of HSA-no linker-GDF15, HSA-GGGGS-GDF15, HSA-GPPGS ate 22, 27, and 21% less food over 24 hours than vehicle treated mice. In summary, these data indicate that fusion of albumin to the N-terminus of GDF15 with various linkers are biologically active.

The amino terminus of GDF15 contains proteolytic (R198) and deamidation sites (N199) that may adversely impact development (e.g., stability) of a therapeutic albumin-GDF15 fusion protein. During purification, we discovered that ˜58% of the HSA-3x4GS-hGDF15(197-308) was proteolysed between residues R198 and N199 and that ˜67% of residue N199 was deamidated. In contrast, no proteolysis or deamindation was observed at these sites when the albumin-GDF15 fusion protein was mutated to HSA-hGDF15(197-308),R198H,N199A. To determine if these sites are required for GDF15 activity, a series of albumin-GDF15 mutants were produced and tested for in vivo activity. Obese mice were treated with a single subcutaneous dose (3 mg/kg s.c.) of HSA-3x4GS-hGDF15(197-308), HSA-hGDF15(197-308),R198H, HSA-hGDF15(197-308),N199E, or HSA-hGDF15(197-308),R198H,N199A. Cumulative food intake over the course of 6 days was reduced by 29% in mice treated with HSA-3x4GS-hGDF15(197-308) compared to vehicle controls. Food intake over the same time period was reduced by 35, 28, and 25% in obese mice treated with HSA-hGDF15(197-308),R198H, HSA-hGDF15(197-308),N199E, or HSA-hGDF15(197-308),R198H,N199A relative to controls. Over the 6 days, the body weight of vehicle treated animals increased by 6.1%, while body weight was reduced by 4.7% in HSA-3x4GS-hGDF15(197-308) treated mice. Body weight was reduced by 5.2, 4.4, and 3.2 in obese mice treated with HSA-hGDF15(197-308),R198H, HSA-hGDF15(197-308),N199E, or HSA-hGDF15(197-308),R198H,N199A, respectively. Thus, fusion proteins containing mutation of these post-translational modification sites in the amino terminus of GDF15 retain biological activity.

As the receptor(s) for GDF15 is unknown, a series of structure-guided site-directed mutants were designed to elucidate domains and residues essential for function and those amenable to modification. GDF15 contains the fingers domain, knuckle domain, wrist domain, the newly discovered N-terminal loop domain, and back-of-hand domain. GDF15 analogs that disrupt the newly discovered amino-terminus region of GDF15, e.g. MSA-GDF15(211-308) and MSA-GDF15 (C203S, C210S), still retain biological activity demonstrating that this loop is not required for activity. The knuckle, finger, and wrist region of TGFbeta superfamily members are known to be important for receptor binding and signaling. To determine if these regions of GDF15 are critical for activity, key surface residues were mutated to a large side-chain containing amino acid, arginine, to attempt to induce a loss of function. MSA-GDF15 fusion proteins containing mutations in GDF15 residues leucine 294 (knuckle), aspartic acid 289 (fingers), glutamine 247 (wrist), and serine 278 (back of hand) were produced and then dosed subcutaneously to obese mice (3 mg/kg s.c.). A single subcutaneous injection of MSA-GDF15 reduced food intake over the course of 7 days by 30% compared to vehicle control. Food intake was also reduced relative to control by the finger region mutant (D289R), the wrist mutant (Q247R), and the back of the hand mutant (S278R) by 22, 14, and 24%, respectively. In contrast, the knuckle region mutant (L294R) increased food intake by 17% relative to control. Over the course of the 7 days, body weight increased in the vehicle and L294R treated mice (2.2 and 6.3% respectively) while body weight decreased in by 6.6, 5.7, 5.7, and 5.4% in the MSA-GDF15, MSA-GDF15 (D289R), MSA-GDF15 (Q247R), and MSA-GDF15 (S278R) treated mice, respectively. These data indicate that L294 and the knuckle region of GDF15 are critical for activity, and likely interact with the GDF15 receptor. Mutations in the other regions of GDF15 are tolerated.

SEQUENCES

Human GDF15 preproprotein (SEQ ID NO: 1)
MPGQELRTVN GSQMLLVLLV LSWLPHGGAL SLAEASRASF PGPSELHSED
SRFRELRKRY
EDLLTRLRAN QSWEDSNTDL VPAPAVRILT PEVRLGSGGH LHLRISRAAL
PEGLPEASRL
HRALFRLSPT ASRSWDVTRP LRRQLSLARP QAPALHLRLS PPPSQSDQLL
AESSSARPQL
ELHLRPQAAR GRRRARARNG DHCPLGPGRC CRLHTVRASL EDLGWADWVL
SPREVQVTMC
IGACPSQFRA ANMHAQIKTS LHRLKPDTVP APCCVPASYN PMVLIQKTDT
GVSLQTYDDL
LAKDCHCI
Human Serum Albumin preproprotein (SEQ ID NO: 2)
>sp|P02768|ALBU_HUMAN Serum albumin OS = Homo sapiens GN = ALB PE = 1 SV = 2
MKWVTFISLLFLFSSAYSRGVFRRDAHKSEVAHRFKDLGEENFKALVLIAFAQYLQQCPF
EDHVKLVNEVTEFAKTCVADESAENCDKSLHTLFGDKLCTVATLRETYGEMADCCAKQEP
ERNECFLQHKDDNPNLPRLVRPEVDVMCTAFHDNEETFLKKYLYEIARRHPYFYAPELLF
FAKRYKAAFTECCQAADKAACLLPKLDELRDEGKASSAKQRLKCASLQKFGERAFKAWAV
ARLSQRFPKAEFAEVSKLVTDLTKVHTECCHGDLLECADDRADLAKYICENQDSISSKLK
ECCEKPLLEKSHCIAEVENDEMPADLPSLAADFVESKDVCKNYAEAKDVFLGMFLYEYAR
RHPDYSVVLLLRLAKTYETTLEKCCAAADPHECYAKVFDEFKPLVEEPQNLIKQNCELFE
QLGEYKFQNALLVRYTKKVPQVSTPTLVEVSRNLGKVGSKCCKHPEAKRMPCAEDYLSVV
LNQLCVLHEKTPVSDRVTKCCTESLVNRRPCFSALEVDETYVPKEFNAETFTFHADICTL
SEKERQIKKQTALVELVKHKPKATKEQLKAVMDDFAAFVEKCCKADDKETCFAEEGKKLV
AASQAALGL
Mouse Igκ chain V-III region MOPC 63 signal peptide (uniprot P01661) (SEQ ID NO: 3):
METDILLLWVLLLWVPGSTG
Human CD8A signal peptide (uniprot P01732) (SEQ ID NO: 4): MALPVTALLLPLALLLHAARP
Modified mating factor alpha-1 signal peptide (uniprot P01149) (SEQ ID NO: 5):
MRFPSIFTAVLFAASSALAAPANTTTEDETAQIPAEAVIDYSDLEGDFDAAALPLSNSTNNGLSSTNTTI
ASIAAKEEGVQLDKR
His₆-hGDF15(197-308)
Open reading frame (SEQ ID NO: 6):

| | |
|---|---|
| atggagacagacacgctgctcctctgggtattgctgctgtgggtaccaggatcc | 54 |
| accggccatcaccaccaccatcatgccagaaacggtgatcattgcccacttgga | 108 |
| cccgggaggtgctgtcggcttcacactgtcagggcatcactcgaagatctcggg | 162 |
| tgggcggactgggtgctttcgcccagagaagtgcaagtcactatgtgcattggt | 216 |
| gcgtgcccgtcgcaattcagagctgccaacatgcatgcccagatcaaaacgagc | 270 |
| ttgcaccggctgaaacccgacacagtccccgctccgtgctgcgtgccggcgtcg | 324 |
| tataaccccatggtcctcatccagaaaaccgatacgggagtgtcattgcagaca | 378 |
| tatgatgaccttttggccaaggattgccactgtatc | 414 |

Expressed protein (SEQ ID NO: 7):

| | |
|---|---|
| HHHHHHARNG DHCPLGPGRC CRLHTVRASL EDLGWADWVL SPREVQVTMC IGACPSQFRA | 60 |
| ANMHAQIKTS LHRLKPDTVP APCCVPASYN PMVLIQKTDT GVSLQTYDDL LAKDCHCI | 118 |

His₈-TEV-hGDF15(197-308)
Open reading frame (SEQ ID NO: 8):

| | |
|---|---|
| atggagacagacacgctgctcctctgggtattgctgctgtgggtaccaggatcc | 54 |
| accggccatcaccaccaccatcatcaccacggcggaagcgagaacctgtacttc | 108 |
| cagggcgccagaaacggtgatcattgcccacttggacccgggaggtgctgtcgg | 162 |
| cttcacactgtcagggcatcactcgaagatctcgggtgggcggactgggtgctt | 216 |
| tcgcccagagaagtgcaagtcactatgtgcattggtgcgtgcccgtcgcaattc | 270 |
| agagctgccaacatgcatgcccagatcaaaacgagcttgcaccggctgaaaccc | 324 |
| gacacagtccccgctccgtgctgcgtgccggcgtcgtataaccccatggtcctc | 378 |
| atccagaaaaccgatacgggagtgtcattgcagacatatgatgaccttttggcc | 432 |
| aaggattgccactgtatc | 450 |

Expressed protein (SEQ ID NO: 9):

| | |
|---|---|
| HHHHHHHHGG SENLYFQGAR NGDHCPLGPG RCCRLHTVRA SLEDLGWADW VLSPREVQVT | 60 |
| MCIGACPSQF RAANMHAQIK TSLHRLKPDT VPAPCCVPAS YNPMVLIQKT DTGVSLQTYD | 120 |
| DLLAKDCHCI | 130 |

hGDF15(197-308) (Yeast expression)
Open reading frame (SEQ ID NO: 10):

| | |
|---|---|
| atgagattcccttccatctttacagcagtgttatttgctgctagttccgcccta | 54 |
| gcagctccagctaacacgactactgaagatgaaacagcccaaatcccagcagaa | 108 |
| gctgttattgactacagcgacttggagggtgacttcgacgcagctgctctcccc | 162 |
| ctttctaattctactaataatggactgagttccacaaatactaccattgcctca | 216 |
| attgccgccaaggaggaaggtgtccaactggacaaaagagctagaaatggtgac | 270 |
| cactgccctttaggtcccggcagatgttgtcgtttgcatactgtgagagcatca | 324 |
| ctggaggatctaggatgggctgattgggtgttgtctccaagggaggttcaggta | 378 |
| actatgtgtataggagcatgcccatcccagttcagggctgcaaacatgcacgct | 432 |
| caaatcaaaacaagccttcatcgtttgaaacctgatacagtaccggcaccatgt | 486 |
| tgtgttccagcttcatataaccctatggtcctgatccaaaagaccgacactggt | 540 |
| gtttcgttgcaaacgtacgatgatttgttggctaaggattgccattgtatt | 591 |

Expressed protein (SEQ ID NO: 11):

| | |
|---|---|
| ARNGDHCPLG PGRCCRLHTV RASLEDLGWA DWVLSPREVQ VTMCIGACPS QFRAANMHAQ | 60 |
| IKTSLHRLKP DTVPAPCCVP ASYNPMVLIQ KTDTGVSLQT YDDLLAKDCH CI | 112 |

hGDF15(197-308) (E. coli expression)
Open reading frame (SEQ ID NO: 12):

| | |
|---|---|
| atgcatcaccatcatcatcaccaaaaacctgttggcgttgaagagccggtctac | 54 |
| gatactgcaggtcgtcctctttttgggaatccgtccgaagtgcacccccagtca | 108 |
| accctcaagcttccccatgaccgcggagaagatgacattgaaacaacgctgcgc | 162 |
| gatctgcctcgtaaaggcgattgtcgctctggaaaccacctaggtccggtgtcg | 216 |
| ggcatttacattaaaccaggtcccgtctattaccaagactacactggtccggtt | 270 |
| taccatcgtgcacctctggaattctttgatgaaacccaatttgaggaaaccact | 324 |
| aaacgtattggccgtgtaaccggttcggacgggaaactgtaccacatctacgtg | 378 |
| gaggttgatggcgagatcctgctgaaacaggcgaagcgcggaacccctcgcacc | 432 |
| ctgaaatggacccgtaacaccactaactgtccactgtgggtcactagttgcgca | 486 |
| cgcaacggtgatcattgtccgctgggtcctggtcgctgctgccgtctgcatacg | 540 |
| gtgcgtgcgagcctggaagatctgggctgggcagattgggtcctgtccccacgc | 594 |
| gaggttcaagtgacgatgtgcattggtgcgtgcccgagccagttccgtgcggcc | 648 |
| aacatgcacgcacagattaagacctctctgcaccgtctgaaaccggacaccgtg | 702 |
| ccggctccgtgttgtgtcccggccagctataatccgatggttctgatccaaaag | 756 |
| accgacaccggcgttagcttgcagacttacgacgatctgttggcgaaagactgt | 810 |
| cactgcatc | 819 |

Expressed protein (Protein1) (SEQ ID NO: 13):

| | |
|---|---|
| MHHHHHHQKP VGVEEPVYDT AGRPLFGNPS EVHPQSTLKL PHDRGEDDIE TTLRDLPRKG | 60 |
| DCRSGNHLGP VSGIYIKPGP VYYQDYTGPV YHRAPLEFFD ETQFEETTKR IGRVTGSDGK | 120 |
| LYHIYVEVDG EILLKQAKRG TPRTLKWTRN TTNCPLWVTS CARNGDHCPL GPGRCCRLHT | 180 |
| VRASLEDLGW ADWVLSPREV QVTMCIGACP SQFRAANMHA QIKTSLHRLK PDTVPAPCCV | 240 |
| PASYNPMVLI QKTDTGVSLQ TYDDLLAKDC HCI | 273 |

GDF15 after Npro auto cleavage (Protein2) (SEQ ID NO: 14):

| | |
|---|---|
| ARNGDHCPLG PGRCCRLHTV RASLEDLGWA DWVLSPREVQ VTMCIGACPS QFRAANMHAQ | 60 |
| IKTSLHRLKP DTVPAPCCVP ASYNPMVLIQ KTDTGVSLQT YDDLLAKDCH CI | 112 |

MSA-hGDF15(197-308)
Open reading frame (SEQ ID NO: 15):

| | |
|---|---|
| Atggagactgataccctgctcctctgggtgctgcttctctgggtccctggctca | 54 |
| Accggcgaagcccacaagtccgagatcgcccatcgctataatgctcttggagaa | 108 |
| Cagcatttcaagggactggtgctgattgccttctcccagtacctccaaaaggcc | 162 |
| Agctatgatgagcacgccaagctcgtccaagaagtcaccgactttgctaagact | 216 |
| Tgtgtggccgacgaaagcgctgccaattgcgataagtcactccatactctcttc | 270 |
| Ggggacaagctgtgcgctattcccaacctccgcgagaattacggtgagctggcc | 324 |
| gactgttgcaccaaacaggagccagagcggaacgagtgcttccttcaacacaaa | 378 |
| gatgacaatccttcactgcctcctttcgaacggcccgaggcagaggcaatgtgc | 432 |
| actagcttcaaggagaacccaaccaccttcatgggacactacctccatgaggtc | 486 |
| gctagacggcatccctacttctatgccccagagcttctgtattatgcagaacag | 540 |
| tacaatgagatcctgacccagtgctgtgctgaggctgataaggagagctgcctg | 594 |
| accccaaagctcgacggagtgaaggaaaaggctcttgtgtccagcgtgcggcag | 648 |
| cgcatgaagtgctcttcaatgcagaagtttggggagcgcgccttcaaagcctgg | 702 |
| gccgtggccagactgtcccagacctttcctaatgctgactttgccgagatcacc | 756 |
| aagctcgctactgacctgaccaaggtcaacaaagagtgttgccacggagatctg | 810 |
| ctcgaatgcgccgacgaccgcgctgagcttgctaagtacatgtgcgaaaaccag | 864 |
| gcaaccatttctagcaagctgcagacctgttgtgataagcctctgctgaagaaa | 918 |
| gcccattgcctcagcgaggtcgaacatgacactatgccggcagacctccccgct | 972 |
| atcgccgctgacttcgtggaggaccaagaagtgtgcaagaattacgccgaggct | 1026 |
| aaggacgtgttccttggtactttcctctacgagtatagccggaggcaccctgac | 1080 |
| tacagcgtgtctcttctgcttcggctcgccaagaagtacgaagccaccctcgaa | 1134 |
| aaatgctgcgccgaagcaaatccgccagcttgttacgggactgtgctggctgag | 1188 |
| tttcagcccctggtggaagagcccaagaacctcgtcaagaccaactgcgacctt | 1242 |
| tacgagaaactgggtgaatacgggtttcagaatgccattctggtgcggtacacc | 1296 |
| cagaaggcaccacaagtgtccaccccaacccttgtcgaggcagcccgcaacctt | 1350 |
| ggacgcgtcgggaccaagtgttgtaccctgcccgaggaccaacgcctgccctgc | 1404 |
| gtcgaggactaccttagcgccattctgaacagagtctgtctgctccatgaaaag | 1458 |
| acccctgtgtctgagcacgtgaccaagtgctgttcaggctcactggtggagagg | 1512 |
| aggccttgcttttctgccctgaccgtggacgaaacctacgtgcccaaggagttc | 1566 |
| aaagctgaaaccttcactttccattcagacatctgtaccctccccgaaaaggaa | 1620 |
| aagcaaatcaagaagcagaccgcccttgctgaactggtgaagcacaagccaaag | 1674 |
| gccaccgccgaacaactcaagactgtgatggacgacttcgctcagttcctcgac | 1728 |
| acttgctgcaaagccgccgacaaagatacctgtttctcaaccgaggggccgaac | 1782 |
| ctggtgactagagccaaggacgccctggccggaggaggtggttctggcggtggt | 1836 |
| ggttccggcggaggaggatctgccaggaatggagatcactgcccactcggaccg | 1890 |
| ggacggtgttgtcgcctgcacactgtgcgcgcatctcttgaggatctgggatgg | 1944 |
| gctgattgggtgctctctcccagagaggtgcaagtcaccatgtgcattggcgcc | 1998 |
| tgcccctcccaattcagggcagctaacatgcatgctcagatcaagactagcctg | 2052 |
| cacaggctgaagcccgacactgtccctgccccatgttgtgtgccggcctcctat | 2106 |
| aacccaatggtcctgatccaaaagaccgataccggagtgtcacttcagacttac | 2160 |
| gacgatctgcttgcaaaagactgccattgcatc | 2193 |

Expressed protein (SEQ ID NO: 16):

| | |
|---|---|
| EAHKSEIAHR YNALGEQHFK GLVLIAFSQY LQKASYDEHA KLVQEVTDFA KTCVADESAA | 60 |
| NCDKSLHTLF GDKLCAIPNL RENYGELADC CTKQEPERNE CFLQHKDDNP SLPPFERPEA | 120 |
| EAMCTSFKEN PTTFMGHYLH EVARRHPYFY APELLYYAEQ YNEILTQCCA EADKESCLTP | 180 |
| KLDGVKEKAL VSSVRQRMKC SSMQKFGERA FKAWAVARLS QTFPNADFAE ITKLATDLTK | 240 |
| VNKECCHGDL LECADDRAEL AKYMCENQAT ISSKLQTCCD KPLLKKAHCL SEVEHDTMPA | 300 |
| DLPAIAADFV EDQEVCKNYA EAKDVFLGTF LYEYSRRHPD YSVSLLLRLA KKYEATLEKC | 360 |
| CAEANPPACY GTVLAEFQPL VEEPKNLVKT NCDLYEKLGE YGFQNAILVR YTQKAPQVST | 420 |
| PTLVEAARNL GRVGTKCCTL PEDQRLPCVE DYLSAILNRV CLLHEKTPVS EHVTKCCSGS | 480 |
| LVERRPCFSA LTVDETYVPK EFKAETFTFH SDICTLPEKE KQIKKQTALA ELVKHKPKAT | 540 |
| AEQLKTVMDD FAQFLDTCCK AADKDTCFST EGPNLVTRAK DALAGGGGSG GGGSGGGGSA | 600 |
| RNGDHCPLGP GRCCRLHTVR ASLEDLGWAD WVLSPREVQV TMCIGACPSQ FRAANMHAQI | 660 |
| KTSLHRLKPD TVPAPCCVPA SYNPMVLIQK TDTGVSLQTY DDLLAKDCHC I | 711 |

MSA-hGDF15(211-308)
Open reading frame (SEQ ID NO: 17):

| | |
|---|---|
| atggagactgatacccttctgctctgggtgcttctgctgtgggtgccaggatcc | 54 |
| accggcgaagcccataagtcggaaatcgcacatcggtacaacgcgctcggggaa | 108 |
| cagcacttcaaaggccttgtcctgatcgcgttctcccaataccttcaaaaggcc | 162 |
| tcgtacgatgaacatgctaagctcgtccaagaggtgaccgacttcgcaaagact | 216 |
| tgtgtggccgatgagtcggcagccaactgcgacaagagcctccacactctcttc | 270 |
| ggagacaagctgtgcgcaattcctaatctgcgcgagaattacggggaactggcg | 324 |
| gactgctgtactaagcaagagccggaacgcaatgagtgcttcctccagcataag | 378 |
| gacgacaacccttccctccctcccttcgaacgcccagaggccgaagcgatgtgt | 432 |
| acctccttcaaggaaaacccgaccacgtttatgggacattacctccacgaagtc | 486 |
| gccagacggcatccctacttctacgcgcctgagctgctctattacgccgaacag | 540 |
| tacaacgagatcctgacgcagtgttgcgctgaggcagacaaggagagctgcttg | 594 |
| accccgaaactcgatggagtgaaggagaaggccctggtgagcagcgtgcgccag | 648 |
| cggatgaagtgctcatcgatgcagaagttcggcgagagagctttcaaggcgtgg | 702 |
| gccgtggccaggctgtcacagacctttccaaacgcggatttcgcagagatcacc | 756 |
| aagctggccactgacctcactaaagtcaacaaggaatgctgccacggagatctc | 810 |
| ttggaatgtgccgatgacagggccgaattggctaagtacatgtgcgaaaatcaa | 864 |
| gctaccattagctcgaagctgcagacgtgctgcgataagccgctgctgaagaag | 918 |
| gctcattgcctgtccgaggtggagcacgacaccatgccagccgacctcccggcc | 972 |
| atcgcagcagattttgtggaggatcaggaagtgtgcaagaattacgcagaagct | 1026 |
| aaggatgtgtttcttgggacttttctctacgagtacagccggagacacccggac | 1080 |
| tatagcgtgtccctgctgctgcgcttggctaagaaatacgaagctacccttgaa | 1134 |
| aaatgctgcgcagaggccaaccctccggcttgctacggaactgtgctggctgag | 1188 |
| ttccagccgctcgtcgaagaaccgaagaatctcgtgaaaacgaactgcgatctg | 1242 |
| tacgagaaattgggagagtatggatttcaaaatgccattctggtccgctacact | 1296 |
| cagaaagctccacaagtctccacgccgaccctggtcgaagcggcgaggaacctt | 1350 |
| ggacgcgtgggaaccaagtgctgtaccctgccggaggaccagcgccttccgtgc | 1404 |
| gtcgaggattacttgtcagcgatcctcaaccgcgtgtgcttgcttcatgaaaag | 1458 |
| actcccgtgtcggaacacgtgacgaagtgctgctccggttcgctggtggaaaga | 1512 |
| cgcccgtgcttctcggccctgactgtggacgaaacctacgtcccaaaagagttc | 1566 |
| aaggctgaaaccttcactttccactcggacatctgcactctccccgaaaaggaa | 1620 |
| aaacagatcaagaagcagactgccctggcagagctggtgaaacacaagcccaag | 1674 |
| gcgacggccgaacagctgaaaaccgtgatggacgactttgcccaattcctcgac | 1728 |
| acttgttgtaaagcagccgataaggacacttgcttctccactgagggccctaac | 1782 |
| ctggtcacccgggctaaggacgcgctcgcgggaggaggtggcagcggaggaggc | 1836 |
| ggtagcggaggcggagggtcatgtcggctgcacaccgtgcgggcatcgcttgaa | 1890 |
| gatttgggatgggccgactgggtgctgtcaccgcgggaagtgcaagtgaccatg | 1944 |
| tgcatcggcgcctgcccgtcgcagtttagagcagcgaatatgcacgcgcaaatc | 1998 |
| aagacttcgctgcacagactgaagccggatactgtccctgcaccatgctgcgtc | 2052 |
| cctgcctcatacaacccaatggtgctgatccagaaaaccgacaccggagtgtcg | 2106 |
| ctccagacttacgacgaccttctggccaaggactgtcattgtatc | 2151 |

Expressed protein (SEQ ID NO: 18):

| | |
|---|---|
| EAHKSEIAHR YNALGEQHFK GLVLIAFSQY LQKASYDEHA KLVQEVTDFA KTCVADESAA | 60 |
| NCDKSLHTLF GDKLCAIPNL RENYGELADC CTKQEPERNE CFLQHKDDNP SLPPFERPEA | 120 |
| EAMCTSFKEN PTTFMGHYLH EVARRHPYFY APELLYYAEQ YNEILTQCCA EADKESCLTP | 180 |
| KLDGVKEKAL VSSVRQRMKC SSMQKFGERA FKAWAVARLS QTFPNADFAE ITKLATDLTK | 240 |
| VNKECCHGDL LECADDRAEL AKYMCENQAT ISSKLQTCCD KPLLKKAHCL SEVEHDTMPA | 300 |
| DLPAIAADFV EDQEVCKNYA EAKDVFLGTF LYEYSRRHPD YSVSLLLRLA KKYEATLEKC | 360 |
| CAEANPPACY GTVLAEFQPL VEEPKNLVKT NCDLYEKLGE YGFQNAILVR YTQKAPQVST | 420 |
| PTLVEAARNL GRVGTKCCTL PEDQRLPCVE DYLSAILNRV CLLHEKTPVS EHVTKCCSGS | 480 |
| LVERRPCFSA LTVDETYVPK EFKAETFTFH SDICTLPEKE KQIKKQTALA ELVKHKPKAT | 540 |
| AEQLKTVMDD FAQFLDTCCK AADKDTCFST EGPNLVTRAK DALAGGGGSG GGGSGGGGSC | 600 |
| RLHTVRASLE DLGWADWVLS PREVQVTMCI GACPSQFRAA NMHAQIKTSL HRLKPDTVPA | 660 |
| PCCVPASYNP MVLIQKTDTG VSLQTYDDLL AKDCHCI | 697 |

HSA(25-609), C34S, N503Q-hGDF15(211-308)
Open reading frame (SEQ ID NO: 19):

| | |
|---|---|
| atggaaactgacactttgctgctttgggttctgctcctttgggtccctggatca | 54 |
| actggtgatgctcacaagtccgaagtggcccaccgtttcaaggatctgggtgag | 108 |
| gaaaacttcaaggctctcgtcctgatcgcatttgcgcagtacctccagcagtcg | 162 |
| ccattcgaggaccatgtgaaactcgtcaacgaagtgaccgagtttgctaagact | 216 |
| tgcgtcgctgacgagtcagcagagaattgtgacaaatccctgcacaccctgttc | 270 |
| ggcgataagctctgcactgtggccaccctccgggaaacttacggcgagatggcg | 324 |
| gattgttgcgcgaaacaggaacccgagcgcaatgagtgtttcctgcagcacaag | 378 |
| gacgacaacccgaacctcccacggctggtgaggccggaagtggacgtcatgtgc | 432 |
| accgcatttcatgacaacgaagagactttcctgaagaagtacctgtacgaaatc | 486 |
| gctcggagacatccgtacttctacgcgccggaactcctcttctttgctaagcgg | 540 |
| tacaaggcagcctttactgaatgctgccaggccgccgacaaagcggcgtgtctg | 594 |
| ctgccgaaactggacgagctgagagatgaaggaaaggctagctcggccaagcag | 648 |
| cggttgaaatgcgcatcgctccaaaagttcggagaaagagctttcaaggcctgg | 702 |
| gcagtggcgcggctctcgcagcgcttccctaaggcagagttcgccgaggtcagc | 756 |
| aagttggtgacggacctgactaaagtccataccgaatgttgccacggagatctg | 810 |
| ctcgaatgcgccgatgaccgggccgacctggcgaagtacatttgtgagaaccaa | 864 |
| gattcaatttcgagcaagttgaaggagtgctgcgaaaagccgttgcttgagaag | 918 |
| tcgcactgcatcgcagaagtcgaaaacgatgagatgcctgccgacttgccgagc | 972 |
| ctggccgccgatttcgtggagagcaaagacgtgtgcaaaaattacgccgaggcc | 1026 |
| aaggacgtgttcctgggaatgttcctgtacgaatatgcgcgacgccacccagac | 1080 |
| tacagcgtggtcctgctgctccgccttgctaaaacttacgaaaccacgctggag | 1134 |
| aaatgctgtgccgcagccgacccacatgagtgctacgcaaaggtgttcgacgag | 1188 |
| tttaaaccccttgtggaagaaccgcagaatctgatcaagcagaactgcgagctg | 1242 |
| ttcgaacaactcggagaatacaagttccagaacgctctgcttgtcagatacacc | 1296 |
| aagaaagtgccgcaagtgtccacgccaaccctggtggaagtctcacgcaacctg | 1350 |
| ggaaaggtcggaagcaagtgctgtaagcatcctgaagcaaagagaatgccatgc | 1404 |
| gcggaggactacctgtccgttgtcctgaatcaactctgcgtgctgcacgagaaa | 1458 |
| actccagtgtcggaccgcgtcaccaagtgttgcacggaatcgctcgtgaatcgc | 1512 |
| aggccgtgcttctccgccctggaagttgatgagacttacgtcccgaaagagttt | 1566 |
| caggccgaaaccttcacctttcacgcggacatctgcactctctctgaaaaggaa | 1620 |
| agacaaatcaagaagcagactgccctggtggagctggtcaagcataaaccaaag | 1674 |
| gcgaccaaggaacagttgaaagccgtgatggacgatttcgctgccttcgtggag | 1728 |
| aagtgctgcaaggccgacgacaaggaaacttgctttgccgaggaaggaaagaaa | 1782 |
| ctggtggccgcatcccaagccgcgctgggactcggaggtggtgggtcgggggga | 1836 |
| gggggctccggcggcggagggtcatgtcgcctccacaccgtgcgggcgtccctg | 1890 |
| gaagatctgggatgggccgattgggtgctgtccccgcgcgaggtgcaagtgact | 1944 |
| atgtgtatcggcgcgtgcccatcacaattcagggcagccaatatgcatgcacag | 1998 |
| atcaaaacctcgctccaccgccttaagccggacaccgtgcccgcgccctgctgc | 2052 |
| gtgcctgcttcctataaccctatggttctgatccaaaagaccgataccggcgtg | 2106 |
| agcctgcagacctacgatgatctcctggccaaggactgccactgtatc | 2154 |

Expressed protein (SEQ ID NO: 20):

| | |
|---|---|
| DAHKSEVAHR FKDLGEENFK ALVLIAFAQY LQQSPFEDHV KLVNEVTEFA KTCVADESAE | 60 |
| NCDKSLHTLF GDKLCTVATL RETYGEMADC CAKQEPERNE CFLQHKDDNP NLPRLVRPEV | 120 |
| DVMCTAFHDN EETFLKKYLY EIARRHPYFY APELLFFAKR YKAAFTECCQ AADKAACLLP | 180 |
| KLDELRDEGK ASSAKQRLKC ASLQKFGERA FKAWAVARLS QRFPKAEFAE VSKLVTDLTK | 240 |
| VHTECCHGDL LECADDRADL AKYICENQDS ISSKLKECCE KPLLEKSHCI AEVENDEMPA | 300 |
| DLPSLAADFV ESKDVCKNYA EAKDVFLGMF LYEYARRHPD YSVVLLLRLA KTYETTLEKC | 360 |
| CAAADPHECY AKVFDEFKPL VEEPQNLIKQ NCELFEQLGE YKFQNALLVR YTKKVPQVST | 420 |
| PTLVEVSRNL GKVGSKCCKH PEAKRMPCAE DYLSVVLNQL CVLHEKTPVS DRVTKCCTES | 480 |
| LVNRRPCFSA LEVDETYVPK EFQAETFTFH ADICTLSEKE RQIKKQTALV ELVKHKPKAT | 540 |
| KEQLKAVMDD FAAFVEKCCK ADDKETCFAE EGKKLVAASQ AALGLGGGGS GGGGSGGGGS | 600 |
| CRLHTVRASL EDLGWADWVL SPREVQVTMC IGACPSQFRA ANMHAQIKTS LHRLKPDTVP | 660 |
| APCCVPASYN PMVLIQKTDT GVSLQTYDDL LAKDCHCI | 698 |

MSA-hGDF15(197-308), C203S, C210S
Open reading frame (SEQ ID NO: 21):

| | |
|---|---|
| atggagactgatacccttctgctctgggtgcttctgctgtgggtgccaggatcc | 54 |
| accggcgaagcccataagtcggaaatcgcacatcggtacaacgcgctcggggaa | 108 |
| cagcacttcaaaggccttgtcctgatcgcgttctcccaataccttcaaaaggcc | 162 |
| tcgtacgatgaacatgctaagctcgtccaagaggtgaccgacttcgcaaagact | 216 |
| tgtgtggccgatgagtcggcagccaactgcgacaagagcctccacactctcttc | 270 |
| ggagacaagctgtgcgcaattcctaatctgcgcgagaattacggggaactggcg | 324 |
| gactgctgtactaagcaagagccggaacgcaatgagtgcttcctccagcataag | 378 |
| gacgacaacccttccctccctcccttcgaacgcccagaggccgaagcgatgtgt | 432 |
| acctccttcaaggaaaacccgaccacgtttatgggacattacctccacgaagtc | 486 |
| gccagacggcatccctacttctacgcgcctgagctgctctattacgccgaacag | 540 |
| tacaacgagatcctgacgcagtgttgcgctgaggcagacaaggagagctgcttg | 594 |
| accccgaaactcgatggagtgaaggagaaggccctggtgagcagcgtgcgccag | 648 |
| cggatgaagtgctcatcgatgcagaagttcggcgagagagctttcaaggcgtgg | 702 |
| gccgtggccaggctgtcacagacctttccaaacgcggatttcgcagagatcacc | 756 |
| aagctggccactgacctcactaaagtcaacaaggaatgctgccacggagatctc | 810 |
| ttggaatgtgccgatgacagggccgaattggctaagtacatgtgcgaaaatcaa | 864 |
| gctaccattagctcgaagctgcagacgtgctgcgataagccgctgctgaagaag | 918 |
| gctcattgcctgtccgaggtggagcacgacaccatgccagccgacctcccggcc | 972 |
| atcgcagcagattttgtggaggatcaggaagtgtgcaagaattacgcagaagct | 1026 |
| aaggatgtgtttcttgggacttttctctacgagtacagccggagacacccggac | 1080 |
| tatagcgtgtccctgctgctgcgcttggctaagaaatacgaagctacccttgaa | 1134 |
| aaatgctgcgcagaggccaaccctccggcttgctacggaactgtgctggctgag | 1188 |
| ttccagccgctcgtcgaagaaccgaagaatctcgtgaaaacgaactgcgatctg | 1242 |
| tacgagaaattgggagagtatggatttcaaaatgccattctggtccgctacact | 1296 |
| cagaaagctccacaagtctccacgccgaccctggtcgaagcggcgaggaacctt | 1350 |
| ggacgcgtgggaaccaagtgctgtaccctgccggaggaccagcgccttccgtgc | 1404 |
| gtcgaggattacttgtcagcgatcctcaaccgcgtgtgcttgcttcatgaaaag | 1458 |
| actcccgtgtcggaacacgtgacgaagtgctgctccggttcgctggtggaaaga | 1512 |
| cgcccgtgcttctcggccctgactgtggacgaaacctacgtcccaaaagagttc | 1566 |
| aaggctgaaaccttcactttccactcggacatctgcactctccccgaaaaggaa | 1620 |
| aaacagatcaagaagcagactgccctggcagagctggtgaaacacaagcccaag | 1674 |
| gcgacggccgaacagctgaaaaccgtgatggacgactttgcccaattcctcgac | 1728 |
| acttgttgtaaagcagccgataaggacacttgcttctccactgagggccctaac | 1782 |
| ctggtcacccgggctaaggacgcgctcgcgggaggaggtggcagcggaggaggc | 1836 |
| ggtagcggaggcggagggagcgctagaaacggcgaccacagcccgttggggcca | 1890 |
| ggtagatcatgtcggctgcacaccgtgcgggcatcgcttgaagatttgggatgg | 1944 |
| gccgactgggtgctgtcaccgcgggaagtgcaagtgaccatgtgcatcggcgcc | 1998 |
| tgcccgtcgcagtttagagcagcgaatatgcacgcgcaaatcaagacttcgctg | 2052 |
| cacagactgaagccggatactgtccctgcaccatgctgcgtccctgcctcatac | 2106 |
| aacccaatggtgctgatccagaaaaccgacaccggagtgtcgctccagacttac | 2160 |
| gacgaccttctggccaaggactgtcattgtatc | 2193 |

Expressed protein (SEQ ID NO: 22):

| | |
|---|---|
| EAHKSEIAHR YNALGEQHFK GLVLIAFSQY LQKASYDEHA KLVQEVTDFA KTCVADESAA | 60 |
| NCDKSLHTLF GDKLCAIPNL RENYGELADC CTKQEPERNE CFLQHKDDNP SLPPFERPEA | 120 |
| EAMCTSFKEN PTTFMGHYLH EVARRHPYFY APELLYYAEQ YNEILTQCCA EADKESCLTP | 180 |
| KLDGVKEKAL VSSVRQRMKC SSMQKFGERA FKAWAVARLS QTFPNADFAE ITKLATDLTK | 240 |
| VNKECCHGDL LECADDRAEL AKYMCENQAT ISSKLQTCCD KPLLKKAHCL SEVEHDTMPA | 300 |
| DLPAIAADFV EDQEVCKNYA EAKDVFLGTF LYEYSRRHPD YSVSLLLRLA KKYEATLEKC | 360 |
| CAEANPPACY GTVLAEFQPL VEEPKNLVKT NCDLYEKLGE YGFQNAILVR YTQKAPQVST | 420 |
| PTLVEAARNL GRVGTKCCTL PEDQRLPCVE DYLSAILNRV CLLHEKTPVS EHVTKCCSGS | 480 |
| LVERRPCFSA LTVDETYVPK EFKAETFTFH SDICTLPEKE KQIKKQTALA ELVKHKPKAT | 540 |
| AEQLKTVMDD FAQFLDTCCK AADKDTCFST EGPNLVTRAK DALAGGGGSG GGGSGGGGSA | 600 |
| RNGDHSPLGP GRSCRLHTVR ASLEDLGWAD WVLSPREVQV TMCIGACPSQ FRAANMHAQI | 660 |
| KTSLHRLKPD TVPAPCCVPA SYNPMVLIQK TDTGVSLQTY DDLLAKDCHC I | 711 |

MSA-hGDF15(197-308), C273S
Open reading frame (SEQ ID NO: 23):

| | |
|---|---|
| atggagactgatacccttctgctctgggtgcttctgctgtgggtgccaggatcc | 54 |
| accggcgaagcccataagtcggaaatcgcacatcggtacaacgcgctcggggaa | 108 |
| cagcacttcaaaggccttgtcctgatcgcgttctcccaataccttcaaaaggcc | 162 |
| tcgtacgatgaacatgctaagctcgtccaagaggtgaccgacttcgcaaagact | 216 |
| tgtgtggccgatgagtcggcagccaactgcgacaagagcctccacactctcttc | 270 |
| ggagacaagctgtgcgcaattcctaatctgcgcgagaattacggggaactggcg | 324 |
| gactgctgtactaagcaagagccggaacgcaatgagtgcttcctccagcataag | 378 |
| gacgacaacccttccctccctcccttcgaacgcccagaggccgaagcgatgtgt | 432 |
| acctccttcaaggaaaacccgaccacgtttatgggacattacctccacgaagtc | 486 |
| gccagacggcatccctacttctacgcgcctgagctgctctattacgccgaacag | 540 |
| tacaacgagatcctgacgcagtgttgcgctgaggcagacaaggagagctgcttg | 594 |
| accccgaaactcgatggagtgaaggagaaggccctggtgagcagcgtgcgccag | 648 |
| cggatgaagtgctcatcgatgcagaagttcggcgagagagctttcaaggcgtgg | 702 |
| gccgtggccaggctgtcacagacctttccaaacgcggatttcgcagagatcacc | 756 |
| aagctggccactgacctcactaaagtcaacaaggaatgctgccacggagatctc | 810 |
| ttggaatgtgccgatgacagggccgaattggctaagtacatgtgcgaaaatcaa | 864 |
| gctaccattagctcgaagctgcagacgtgctgcgataagccgctgctgaagaag | 918 |
| gctcattgcctgtccgaggtggagcacgacaccatgccagccgacctcccggcc | 972 |
| atcgcagcagattttgtggaggatcaggaagtgtgcaagaattacgcagaagct | 1026 |
| aaggatgtgtttcttgggacttttctctacgagtacagccggagacacccggac | 1080 |
| tatagcgtgtccctgctgctgcgcttggctaagaaatacgaagctacccttgaa | 1134 |
| aaatgctgcgcagaggccaaccctccggcttgctacggaactgtgctggctgag | 1188 |
| ttccagccgctcgtcgaagaaccgaagaatctcgtgaaaacgaactgcgatctg | 1242 |
| tacgagaaattgggagagtatggatttcaaaatgccattctggtccgctacact | 1296 |
| cagaaagctccacaagtctccacgccgaccctggtcgaagcggcgaggaacctt | 1350 |
| ggacgcgtgggaaccaagtgctgtaccctgccggaggaccagcgccttccgtgc | 1404 |
| gtcgaggattacttgtcagcgatcctcaaccgcgtgtgcttgcttcatgaaaag | 1458 |
| actcccgtgtcggaacacgtgacgaagtgctgctccggttcgctggtggaaaga | 1512 |
| cgcccgtgcttctcggccctgactgtggacgaaacctacgtcccaaaagagttc | 1566 |
| aaggctgaaaccttcactttccactcggacatctgcactctccccgaaaaggaa | 1620 |
| aaacagatcaagaagcagactgccctggcagagctggtgaaacacaagcccaag | 1674 |
| gcgacggccgaacagctgaaaaccgtgatggacgactttgcccaattcctcgac | 1728 |
| acttgttgtaaagcagccgataaggacacttgcttctccactgagggccctaac | 1782 |
| ctggtcacccgggctaaggacgcgctcgcgggaggaggtggcagcggaggaggc | 1836 |
| ggtagcggaggcggagggagcgctagaaacggcgaccactgtccactggggcca | 1890 |
| ggtcggtgctgtcggctgcacaccgtgcgggcatcgcttgaagatttgggatgg | 1944 |
| gccgactgggtgctgtcaccgcgggaagtgcaagtgaccatgtgcatcggcgcc | 1998 |
| tgcccgtcgcagtttagagcagcgaatatgcacgcgcaaatcaagacttcgctg | 2052 |
| cacagactgaagccggatactgtccctgcaccatcatgcgtccctgcctcatac | 2106 |
| aacccaatggtgctgatccagaaaaccgacaccggagtgtcgctccagacttac | 2160 |
| gacgaccttctggccaaggactgtcattgtatc | 2193 |

Expressed protein (SEQ ID NO: 24):

| | |
|---|---|
| EAHKSEIAHR YNALGEQHFK GLVLIAFSQY LQKASYDEHA KLVQEVTDFA KTCVADESAA | 60 |
| NCDKSLHTLF GDKLCAIPNL RENYGELADC CTKQEPERNE CFLQHKDDNP SLPPFERPEA | 120 |
| EAMCTSFKEN PTTFMGHYLH EVARRHPYFY APELLYYAEQ YNEILTQCCA EADKESCLTP | 180 |
| KLDGVKEKAL VSSVRQRMKC SSMQKFGERA FKAWAVARLS QTFPNADFAE ITKLATDLTK | 240 |
| VNKECCHGDL LECADDRAEL AKYMCENQAT ISSKLQTCCD KPLLKKAHCL SEVEHDTMPA | 300 |
| DLPAIAADFV EDQEVCKNYA EAKDVFLGTF LYEYSRRHPD YSVSLLLRLA KKYEATLEKC | 360 |
| CAEANPPACY GTVLAEFQPL VEEPKNLVKT NCDLYEKLGE YGFQNAILVR YTQKAPQVST | 420 |
| PTLVEAARNL GRVGTKCCTL PEDQRLPCVE DYLSAILNRV CLLHEKTPVS EHVTKCCSGS | 480 |
| LVERRPCFSA LTVDETYVPK EFKAETFTFH SDICTLPEKE KQIKKQTALA ELVKHKPKAT | 540 |
| AEQLKTVMDD FAQFLDTCCK AADKDTCFST EGPNLVTRAK DALAGGGGSG GGGSGGGGSA | 600 |
| RNGDHCPLGP GRCCRLHTVR ASLEDLGWAD WVLSPREVQV TMCIGACPSQ FRAANMHAQI | 660 |
| KTSLHRLKPD TVPAPSCVPA SYNPMVLIQK TDTGVSLQTY DDLLAKDCHC I | 711 |

HSA-3x4GS-hGDF15(197-308)
Open reading frame (SEQ ID NO: 25):

| | |
|---|---|
| atggaaaccgatactctgctgctgtgggtgcttcttctttgggtgccgggatca | 54 |
| accggcgatgcccacaagtcggaggtggcccatcggtttaaggacctcggggag | 108 |
| gagaacttcaaagccctggtcctcatcgccttcgcccaatacctccagcagtgt | 162 |
| ccattcgaagatcacgtgaagctcgtgaacgaagtgactgaatttgccaagact | 216 |
| tgtgtcgcagacgaaagcgccgaaaactgcgacaagtcgttgcatactctcttc | 270 |
| ggggataagctgtgcactgtcgcaacccttagagagacttacggtgaaatggct | 324 |
| gattgctgcgccaaacaagagccggagcgcaacgagtgcttcctccaacataag | 378 |
| gacgacaaccccaacctcccacgcctggtgcggcctgaggtcgacgtcatgtgc | 432 |
| accgctttccatgacaatgaggagacttttctcaagaagtatctgtacgagatc | 486 |
| gcccggaggcacccatacttttatgcaccggagctccttttcttcgctaagcgg | 540 |
| tacaaggcggcgttcactgaatgctgtcaggcagcagacaaggcagcatgcctc | 594 |
| ctgccgaaactggacgaacttcgcgacgagggtaaagcgtcgtccgccaagcag | 648 |
| cgccttaagtgcgcctcgttgcagaagtttggtgaacgcgcattcaaagcgtgg | 702 |
| gccgtcgcaagactttcgcagcggttcccaaaagcggagtttgccgaggtgtcc | 756 |
| aaactggtcaccgacctgaccaaggtccacaccgagtgctgccacggcgatctg | 810 |
| ctcgaatgcgccgacgaccgggctgatctcgcaaagtacatttgcgagaaccaa | 864 |
| gactcgatctcgtcaaaactgaaggaatgctgcgagaagccgctgttggaaaag | 918 |
| agccattgtatcgccgaagtggagaacgatgaaatgcctgctgatctgccaagc | 972 |
| ctcgccgcagactttgtggagagcaaagacgtgtgcaagaactacgccgaagcg | 1026 |
| aaggacgtgtttctcgggatgttcctctacgagtacgcgcgcaggcaccctgac | 1080 |
| tactcagtggtcctgctgttgcggctggccaaaacttacgaaaccaccctcgaa | 1134 |
| aagtgctgcgcggctgccgatccacatgaatgctacgcaaaggtgttcgatgaa | 1188 |
| tttaagcctctggtggaggaaccacagaacctgatcaagcaaaattgtgaactg | 1242 |
| tttgaacagctgggagagtacaaatttcagaatgccctgctggtcagatacact | 1296 |
| aagaaggtgccccaagtctccactccaaccctcgtggaggtgtcacggaatctc | 1350 |
| ggcaaagtgggcagcaaatgctgtaagcacccggaagcaaagaggatgccctgc | 1404 |
| gctgaagattacctgtccgtggtgctgaatcagctttgtgtgctgcacgaaaag | 1458 |
| acgcctgtctccgaccgggtgaccaagtgctgtaccgaatcgctcgtgaatcgc | 1512 |
| agaccctgcttctccgctctcgaagtggacgaaacttacgtcccgaaggagttc | 1566 |
| aatgcggaaaccttcaccttccacgcggacatctgtaccctgagcgaaaaagag | 1620 |
| cggcagatcaagaaacagactgccctggtggaactggtgaagcacaagccgaag | 1674 |
| gcaacgaaggagcagctgaaggcggtgatggatgactttgcagccttcgtggaa | 1728 |
| aagtgttgcaaggcagatgataaagaaacctgtttcgcggaagaggggaagaag | 1782 |
| ttggtggctgccagccaggccgctctcggactgggaggtggaggatcaggaggc | 1836 |
| ggaggctccggaggaggaggctcggctcgcaatggcgatcattgcccgctcgga | 1890 |
| ccgggacgctgctgcagactgcataccgtccgcgcttccttggaagatctggga | 1944 |
| tgggcggattgggtgttgtcaccaagagaggtgcaagtgacgatgtgtatcggt | 1998 |
| gcgtgcccttcacagttccgcgctgcgaacatgcatgcccaaatcaagaccagc | 2052 |
| ctgcaccggctgaagccggacactgtcccagctccatgttgcgtgcccgcatcg | 2106 |
| tacaacccgatggtgctcatccagaaaactgacactggagtctcactgcaaacg | 2160 |
| tacgacgatttgctcgccaaagattgccactgcatt | 2196 |

Expressed protein (SEQ ID NO: 26):

| | |
|---|---|
| DAHKSEVAHR FKDLGEENFK ALVLIAFAQY LQQCPFEDHV KLVNEVTEFA KTCVADESAE | 60 |
| NCDKSLHTLF GDKLCTVATL RETYGEMADC CAKQEPERNE CFLQHKDDNP NLPRLVRPEV | 120 |
| DVMCTAFHDN EETFLKKYLY EIARRHPYFY APELLFFAKR YKAAFTECCQ AADKAACLLP | 180 |
| KLDELRDEGK ASSAKQRLKC ASLQKFGERA FKAWAVARLS QRFPKAEFAE VSKLVTDLTK | 240 |
| VHTECCHGDL LECADDRADL AKYICENQDS ISSKLKECCE KPLLEKSHCI AEVENDEMPA | 300 |
| DLPSLAADFV ESKDVCKNYA EAKDVFLGMF LYEYARRHPD YSVVLLLRLA KTYETTLEKC | 360 |
| CAAADPHECY AKVFDEFKPL VEEPQNLIKQ NCELFEQLGE YKFQNALLVR YTKKVPQVST | 420 |
| PTLVEVSRNL GKVGSKCCKH PEAKRMPCAE DYLSVVLNQL CVLHEKTPVS DRVTKCCTES | 480 |
| LVNRRPCFSA LEVDETYVPK EFNAETFTFH ADICTLSEKE RQIKKQTALV ELVKHKPKAT | 540 |
| KEQLKAVMDD FAAFVEKCCK ADDKETCFAE EGKKLVAASQ AALGLGGGGS GGGGSGGGGS | 600 |
| ARNGDHCPLG PGRCCRLHTV RASLEDLGWA DWVLSPREVQ VTMCIGACPS QFRAANMHAQ | 660 |
| IKTSLHRLKP DTVPAPCCVP ASYNPMVLIQ KTDTGVSLQT YDDLLAKDCH CI | 712 |

HSA-GGGGS-hGDF15(197-308)
Open reading frame (SEQ ID NO: 27):

| | |
|---|---|
| atggccctccctgtcaccgccctgctgcttccgctggctcttctgctccacgcc | 54 |
| gctcggcccgatgctcataaatcagaagtggcgcacagattcaaggacctcgga | 108 |
| gaagaaaactttaaagcactggtgctgatcgccttcgcacaatacttgcagcag | 162 |
| tgcccgttcgaagatcacgtgaaactggtcaacgaagtgaccgagttcgctaag | 216 |
| acctgtgtcgctgacgagagcgcggaaaactgcgacaagtcccttcacacgctg | 270 |
| ttcggcgataagctctgcacggtcgcgactctgagggaaacctacggagagatg | 324 |
| gcagattgctgtgcaaagcaggaacctgagaggaacgaatgtttcctgcaacat | 378 |
| aaggacgacaacccaaatcttccgcgcctcgtgcgtccggaggtggacgtgatg | 432 |
| tgcacggccttccatgataatgaggaaactttcctgaaaaagtacctctacgaa | 486 |
| atcgcccggagacacccgtatttctacgccccggagcttctgttcttcgcaaag | 540 |
| cgctacaaggcggcttttactgagtgctgccaagctgccgacaaagccgcatgc | 594 |
| ctgctgccaaagctcgatgaactcagggacgagggaaaggcatcctccgcaaag | 648 |
| cagcgcctgaaatgcgcctcactgcaaaagtttggagaacgcgcattcaaggcc | 702 |
| tgggcggtggcccggctcagccagagattccccaaggccgagtttgccgaggtg | 756 |
| tccaagctcgttactgatctgaccaaagtccacaccgaatgctgtcatggagat | 810 |
| cttttggagtgcgccgacgacagagcggacctggccaagtacatctgcgaaaac | 864 |
| caggattcgatctcatctaagctcaaggagtgctgcgaaaaacccctgttggaa | 918 |
| aagtcgcactgtattgcggaagtggagaacgacgagatgcctgcagacttgccg | 972 |
| tcactggcggctgacttcgtggagtcgaaggacgtgtgcaaaaactacgcggaa | 1026 |
| gcgaaggatgtctttctgggaatgttcctgtacgaatacgcacggcgccatccg | 1080 |
| gactactcagttgtgctgttgctccgccttgctaagacttacgaaactaccttg | 1134 |
| gagaaatgctgcgccgccgccgatcctcacgaatgttacgcaaaagtgttcgac | 1188 |
| gagtttaagcctctcgtggaagaacctcagaatctgatcaagcagaactgtgaa | 1242 |
| ctgttcgagcagctcggggaatacaagttccagaatgcgctgctcgtccggtat | 1296 |
| actaagaaagtgccacaagtgtccaccccgactctggtcgaagtgtcgcgcaat | 1350 |
| ctggggaaagtcggatcgaagtgctgcaagcatccggaggcgaaacgaatgccg | 1404 |
| tgcgcggaggattacctgtcggtggtgctgaaccagctctgcgtgctgcatgaa | 1458 |
| aagaccccggtgtccgaccgggtcaccaagtgttgcactgagtccctcgtgaac | 1512 |
| cggcgcccttgcttctcggccctcgaagtcgatgagacttacgtgccaaaagag | 1566 |
| tttaatgccgaaaccttcacctttcacgctgacatctgcactttgagcgaaaag | 1620 |
| gaaagacagattaagaagcagacggccctggtggaactcgtcaaacataaaccc | 1674 |
| aaagctacgaaagagcagctgaaagcagttatggacgatttcgccgctttcgtg | 1728 |
| gaaaaatgctgcaaggccgacgataaggaaacttgtttcgccgaggaggggaag | 1782 |
| aagctggtcgcagcaagccaagccgctctgggtcttggcggtggaggcagcgcg | 1836 |
| aggaatggcgaccactgcccattgggaccgggacggtgttgcagactccacact | 1890 |
| gtccgggcttcactcgaggacctgggttgggccgactgggtgctgtcgccccgg | 1944 |
| gaagtccaggtcaccatgtgcatcggagcgtgcccgagccaatttcgcgccgcg | 1998 |
| aacatgcacgcccagatcaagacctcgctgcaccgcctgaagcctgacaccgtg | 2052 |
| ccagccccctgctgtgtgccggcctcctacaacccaatggtgctcatccaaaag | 2106 |
| accgataccggcgtgagcctgcaaacttacgatgatcttctggccaaggactgt | 2160 |
| cactgcatc | 2169 |

Expressed protein (SEQ ID NO: 28):

| | |
|---|---|
| DAHKSEVAHR FKDLGEENFK ALVLIAFAQY LQQCPFEDHV KLVNEVTEFA KTCVADESAE | 60 |
| NCDKSLHTLF GDKLCTVATL RETYGEMADC CAKQEPERNE CFLQHKDDNP NLPRLVRPEV | 120 |
| DVMCTAFHDN EETFLKKYLY EIARRHPYFY APELLFFAKR YKAAFTECCQ AADKAACLLP | 180 |
| KLDELRDEGK ASSAKQRLKC ASLQKFGERA FKAWAVARLS QRFPKAEFAE VSKLVTDLTK | 240 |
| VHTECCHGDL LECADDRADL AKYICENQDS ISSKLKECCE KPLLEKSHCI AEVENDEMPA | 300 |
| DLPSLAADFV ESKDVCKNYA EAKDVFLGMF LYEYARRHPD YSVVLLLRLA KTYETTLEKC | 360 |
| CAAADPHECY AKVFDEFKPL VEEPQNLIKQ NCELFEQLGE YKFQNALLVR YTKKVPQVST | 420 |
| PTLVEVSRNL GKVGSKCCKH PEAKRMPCAE DYLSVVLNQL CVLHEKTPVS DRVTKCCTES | 480 |
| LVNRRPCFSA LEVDETYVPK EFNAETFTFH ADICTLSEKE RQIKKQTALV ELVKHKPKAT | 540 |
| KEQLKAVMDD FAAFVEKCCK ADDKETCFAE EGKKLVAASQ AALGLGGGGS ARNGDHCPLG | 600 |
| PGRCCRLHTV RASLEDLGWA DWVLSPREVQ VTMCIGACPS QFRAANMHAQ IKTSLHRLKP | 660 |
| DTVPAPCCVP ASYNPMVLIQ KTDTGVSLQT YDDLLAKDCH CI | 702 |

HSA-GPPGS-hGDF15(197-308)
Open reading frame (SEQ ID NO: 29):

| | |
|---|---|
| atggccctccctgtcaccgccctgctgcttccgctggctcttctgctccacgcc | 54 |
| gctcggcccgatgctcataaatcagaagtggcgcacagattcaaggacctcgga | 108 |
| gaagaaaactttaaagcactggtgctgatcgccttcgcacaatacttgcagcag | 162 |
| tgcccgttcgaagatcacgtgaaactggtcaacgaagtgaccgagttcgctaag | 216 |
| acctgtgtcgctgacgagagcgcggaaaactgcgacaagtcccttcacacgctg | 270 |
| ttcggcgataagctctgcacggtcgcgactctgagggaaacctacggagagatg | 324 |
| gcagattgctgtgcaaagcaggaacctgagaggaacgaatgtttcctgcaacat | 378 |
| aaggacgacaacccaaatcttccgcgcctcgtgcgtccggaggtggacgtgatg | 432 |
| tgcacggccttccatgataatgaggaaactttcctgaaaaagtacctctacgaa | 486 |
| atcgcccggagacacccgtatttctacgccccggagcttctgttcttcgcaaag | 540 |
| cgctacaaggcggcttttactgagtgctgccaagctgccgacaaagccgcatgc | 594 |
| ctgctgccaaagctcgatgaactcagggacgagggaaaggcatcctccgcaaag | 648 |
| cagcgcctgaaatgcgcctcactgcaaaagtttggagaacgcgcattcaaggcc | 702 |
| tgggcggtggcccggctcagccagagattccccaaggccgagtttgccgaggtg | 756 |
| tccaagctcgttactgatctgaccaaagtccacaccgaatgctgtcatggagat | 810 |
| cttttggagtgcgccgacgacagagcggacctggccaagtacatctgcgaaaac | 864 |
| caggattcgatctcatctaagctcaaggagtgctgcgaaaaacccctgttggaa | 918 |
| aagtcgcactgtattgcggaagtggagaacgacgagatgcctgcagacttgccg | 972 |
| tcactggcggctgacttcgtggagtcgaaggacgtgtgcaaaaactacgcggaa | 1026 |
| gcgaaggatgtctttctgggaatgttcctgtacgaatacgcacggcgccatccg | 1080 |
| gactactcagttgtgctgttgctccgccttgctaagacttacgaaactaccttg | 1134 |
| gagaaatgctgcgccgccgccgatcctcacgaatgttacgcaaaagtgttcgac | 1188 |
| gagtttaagcctctcgtggaagaacctcagaatctgatcaagcagaactgtgaa | 1242 |
| ctgttcgagcagctcggggaatacaagttccagaatgcgctgctcgtccggtat | 1296 |
| actaagaaagtgccacaagtgtccaccccgactctggtcgaagtgtcgcgcaat | 1350 |
| ctggggaaagtcggatcgaagtgctgcaagcatccggaggcgaaacgaatgccg | 1404 |
| tgcgcggaggattacctgtcggtggtgctgaaccagctctgcgtgctgcatgaa | 1458 |
| aagaccccggtgtccgaccgggtcaccaagtgttgcactgagtccctcgtgaac | 1512 |
| cggcgcccttgcttctcggccctcgaagtcgatgagacttacgtgccaaaagag | 1566 |
| tttaatgccgaaaccttcacctttcacgctgacatctgcactttgagcgaaaag | 1620 |
| gaaagacagattaagaagcagacggccctggtggaactcgtcaaacataaaccc | 1674 |
| aaagctacgaaagagcagctgaaagcagttatggacgatttcgccgctttcgtg | 1728 |
| gaaaaatgctgcaaggccgacgataaggaaacttgtttcgccgaggaggggaag | 1782 |
| aagctggtcgcagcaagccaagccgctctgggtcttggcccaccgggcagcgcg | 1836 |
| aggaatggcgaccactgcccattgggaccgggacggtgttgcagactccacact | 1890 |
| gtccgggcttcactcgaggacctgggttgggccgactgggtgctgtcgccccgg | 1944 |
| gaagtccaggtcaccatgtgcatcggagcgtgcccgagccaatttcgcgccgcg | 1998 |
| aacatgcacgcccagatcaagacctcgctgcaccgcctgaagcctgacaccgtg | 2052 |
| ccagccccctgctgtgtgccggcctcctacaacccaatggtgctcatccaaaag | 2106 |
| accgataccggcgtgagcctgcaaacttacgatgatcttctggccaaggactgt | 2160 |
| cactgcatc | 2169 |

Expressed protein (SEQ ID NO: 30):

| | |
|---|---|
| DAHKSEVAHR FKDLGEENFK ALVLIAFAQY LQQCPFEDHV KLVNEVTEFA KTCVADESAE | 60 |
| NCDKSLHTLF GDKLCTVATL RETYGEMADC CAKQEPERNE CFLQHKDDNP NLPRLVRPEV | 120 |
| DVMCTAFHDN EETFLKKYLY EIARRHPYFY APELLFFAKR YKAAFTECCQ AADKAACLLP | 180 |
| KLDELRDEGK ASSAKQRLKC ASLQKFGERA FKAWAVARLS QRFPKAEFAE VSKLVTDLTK | 240 |
| VHTECCHGDL LECADDRADL AKYICENQDS ISSKLKECCE KPLLEKSHCI AEVENDEMPA | 300 |
| DLPSLAADFV ESKDVCKNYA EAKDVFLGMF LYEYARRHPD YSVVLLLRLA KTYETTLEKC | 360 |
| CAAADPHECY AKVFDEFKPL VEEPQNLIKQ NCELFEQLGE YKFQNALLVR YTKKVPQVST | 420 |
| PTLVEVSRNL GKVGSKCCKH PEAKRMPCAE DYLSVVLNQL CVLHEKTPVS DRVTKCCTES | 480 |
| LVNRRPCFSA LEVDETYVPK EFNAETFTFH ADICTLSEKE RQIKKQTALV ELVKHKPKAT | 540 |
| KEQLKAVMDD FAAFVEKCCK ADDKETCFAE EGKKLVAASQ AALGLGPPGS ARNGDHCPLG | 600 |
| PGRCCRLHTV RASLEDLGWA DWVLSPREVQ VTMCIGACPS QFRAANMHAQ IKTSLHRLKP | 660 |
| DTVPAPCCVP ASYNPMVLIQ KTDTGVSLQT YDDLLAKDCH CI | 702 |

HSA-hGDF15(197-308)(no linker)
Open reading frame (SEQ ID NO: 31):

| | |
|---|---|
| atggccctccctgtcaccgccctgctgcttccgctggctcttctgctccacgcc | 54 |
| gctcggcccgatgctcataaatcagaagtggcgcacagattcaaggacctcgga | 108 |
| gaagaaaactttaaagcactggtgctgatcgccttcgcacaatacttgcagcag | 162 |
| tgcccgttcgaagatcacgtgaaactggtcaacgaagtgaccgagttcgctaag | 216 |
| acctgtgtcgctgacgagagcgcggaaaactgcgacaagtcccttcacacgctg | 270 |
| ttcggcgataagctctgcacggtcgcgactctgagggaaacctacggagagatg | 324 |
| gcagattgctgtgcaaagcaggaacctgagaggaacgaatgtttcctgcaacat | 378 |
| aaggacgacaacccaaatcttccgcgcctcgtgcgtccggaggtggacgtgatg | 432 |
| tgcacggccttccatgataatgaggaaactttcctgaaaaagtacctctacgaa | 486 |
| atcgcccggagacacccgtatttctacgccccggagcttctgttcttcgcaaag | 540 |
| cgctacaaggcggcttttactgagtgctgccaagctgccgacaaagccgcatgc | 594 |
| ctgctgccaaagctcgatgaactcagggacgagggaaaggcatcctccgcaaag | 648 |
| cagcgcctgaaatgcgcctcactgcaaaagtttggagaacgcgcattcaaggcc | 702 |
| tgggcggtggcccggctcagccagagattccccaaggccgagtttgccgaggtg | 756 |
| tccaagctcgttactgatctgaccaaagtccacaccgaatgctgtcatggagat | 810 |
| cttttggagtgcgccgacgacagagcggacctggccaagtacatctgcgaaaac | 864 |
| caggattcgatctcatctaagctcaaggagtgctgcgaaaaacccctgttggaa | 918 |
| aagtcgcactgtattgcggaagtggagaacgacgagatgcctgcagacttgccg | 972 |
| tcactggcggctgacttcgtggagtcgaaggacgtgtgcaaaaactacgcggaa | 1026 |
| gcgaaggatgtctttctgggaatgttcctgtacgaatacgcacggcgccatccg | 1080 |
| gactactcagttgtgctgttgctccgccttgctaagacttacgaaactaccttg | 1134 |
| gagaaatgctgcgccgccgccgatcctcacgaatgttacgcaaaagtgttcgac | 1188 |
| gagtttaagcctctcgtggaagaacctcagaatctgatcaagcagaactgtgaa | 1242 |
| ctgttcgagcagctcggggaatacaagttccagaatgcgctgctcgtccggtat | 1296 |
| actaagaaagtgccacaagtgtccaccccgactctggtcgaagtgtcgcgcaat | 1350 |
| ctggggaaagtcggatcgaagtgctgcaagcatccggaggcgaaacgaatgccg | 1404 |
| tgcgcggaggattacctgtcggtggtgctgaaccagctctgcgtgctgcatgaa | 1458 |
| aagaccccggtgtccgaccgggtcaccaagtgttgcactgagtccctcgtgaac | 1512 |
| cggcgcccttgcttctcggccctcgaagtcgatgagacttacgtgccaaaagag | 1566 |
| tttaatgccgaaaccttcacctttcacgctgacatctgcactttgagcgaaaag | 1620 |
| gaaagacagattaagaagcagacggccctggtggaactcgtcaaacataaaccc | 1674 |
| aaagctacgaaagagcagctgaaagcagttatggacgatttcgccgctttcgtg | 1728 |
| gaaaaatgctgcaaggccgacgataaggaaacttgtttcgccgaggaggggaag | 1782 |
| aagctggtcgcagcaagccaagccgctctgggtcttgcgaggaatggcgaccac | 1836 |
| tgcccattgggaccgggacggtgttgcagactccacactgtccgggcttcactc | 1890 |
| gaggacctgggttgggccgactgggtgctgtcgccccgggaagtccaggtcacc | 1944 |
| atgtgcatcggagcgtgcccgagccaatttcgcgccgcgaacatgcacgcccag | 1998 |
| atcaagacctcgctgcaccgcctgaagcctgacaccgtgccagccccctgctgt | 2052 |
| gtgccggcctcctacaacccaatggtgctcatccaaaagaccgataccggcgtg | 2106 |
| agcctgcaaacttacgatgatcttctggccaaggactgtcactgcatc | 2154 |

Expressed protein (SEQ ID NO: 32):

| | |
|---|---|
| DAHKSEVAHR FKDLGEENFK ALVLIAFAQY LQQCPFEDHV KLVNEVTEFA KTCVADESAE | 60 |
| NCDKSLHTLF GDKLCTVATL RETYGEMADC CAKQEPERNE CFLQHKDDNP NLPRLVRPEV | 120 |
| DVMCTAFHDN EETFLKKYLY EIARRHPYFY APELLFFAKR YKAAFTECCQ AADKAACLLP | 180 |
| KLDELRDEGK ASSAKQRLKC ASLQKFGERA FKAWAVARLS QRFPKAEFAE VSKLVTDLTK | 240 |
| VHTECCHGDL LECADDRADL AKYICENQDS ISSKLKECCE KPLLEKSHCI AEVENDEMPA | 300 |
| DLPSLAADFV ESKDVCKNYA EAKDVFLGMF LYEYARRHPD YSVVLLLRLA KTYETTLEKC | 360 |
| CAAADPHECY AKVFDEFKPL VEEPQNLIKQ NCELFEQLGE YKFQNALLVR YTKKVPQVST | 420 |
| PTLVEVSRNL GKVGSKCCKH PEAKRMPCAE DYLSVVLNQL CVLHEKTPVS DRVTKCCTES | 480 |
| LVNRRPCFSA LEVDETYVPK EFNAETFTFH ADICTLSEKE RQIKKQTALV ELVKHKPKAT | 540 |
| KEQLKAVMDD FAAFVEKCCK ADDKETCFAE EGKKLVAASQ AALGLARNGD HCPLGPGRCC | 600 |
| RLHTVRASLE DLGWADWVLS PREVQVTMCI GACPSQFRAA NMHAQIKTSL HRLKPDTVPA | 660 |
| PCCVPASYNP MVLIQKTDTG VSLQTYDDLL AKDCHCI | 697 |

MSA_Domain1-3x4GS-hGDF15
Open reading frame (SEQ ID NO: 33):

| | |
|---|---|
| atggccctccctgtcaccgccctgctgcttccgctggctcttctgctccacgcc | 54 |
| gctcggcccgaagctcataagtcagaaatcgcccatagatacaacgacctcggg | 108 |
| gaacagcactttaaaggactcgtgttgattgcattcagccagtacctccaaaag | 162 |
| tgcagctacgacgagcatgcgaagctggtgcaggaagtcaccgacttcgccaaa | 216 |
| acttgcgtcgctgatgagtcggcggcaaactgcgacaaatcgctccacaccctg | 270 |
| tttggcgataagctgtgtgcgatcccgaatcttcgagagaattacggagaactt | 324 |
| gcagactgctgcaccaagcaggaaccggaacgcaacgagtgcttcctccaacac | 378 |
| aaggatgacaacccatctctgccccctttcgaacggccggaggcggaagccatg | 432 |
| tgcactagctttaaggagaatccaactacgttcatggggcattacctccacgag | 486 |
| gtcgccaggcggcatccatacttctacgccccggaactgctgtactatgccgag | 540 |
| cagtacaacgaaatcctgacgcagtgctgtgccgaggctgataaggaatcatgc | 594 |
| ctgaccccaaagctggacggagtgaaagaaaaggcgctcgtgtcgtccgtgaga | 648 |
| caacgcggtggaggaggctccggcggcggaggctcgggagggggaggttcagca | 702 |
| cggaacggcgaccactgccctttggggccgggacgctgttgccggcttcacact | 756 |
| gtgcgcgcgtccctcgaggatttgggatgggcagattgggtgctgagcccgaga | 810 |
| gaggtccaggtcaccatgtgtatcggtgcctgcccgagccagttcagggctgcc | 864 |
| aacatgcacgcgcagatcaaaacttcgctgcatcgcctgaaaccagacaccgtt | 918 |
| ccggcaccctgttgcgtgcctgcctcctacaatcctatggtgctgattcaaaag | 972 |
| accgacaccggagtgtccctgcaaacttacgacgatctgctcgccaaggactgc | 1026 |
| cactgtatc | 1035 |

Expressed protein (SEQ ID NO: 34):

| | |
|---|---|
| EAHKSEIAHR YNDLGEQHFK GLVLIAFSQY LQKCSYDEHA KLVQEVTDFA KTCVADESAA | 60 |
| NCDKSLHTLF GDKLCAIPNL RENYGELADC CTKQEPERNE CFLQHKDDNP SLPPFERPEA | 120 |
| EAMCTSFKEN PTTFMGHYLH EVARRHPYFY APELLYYAEQ YNEILTQCCA EADKESCLTP | 180 |
| KLDGVKEKAL VSSVRQRGGG GSGGGGSGGG GSARNGDHCP LGPGRCCRLH TVRASLEDLG | 240 |
| WADWVLSPRE VQVTMCIGAC PSQFRAANMH AQIKTSLHRL KPDTVPAPCC VPASYNPMVL | 300 |
| IQKTDTGVSL QTYDDLLAKD CHCI | |

hFc-3x4GS-hGDF15(197-308)
Open reading frame (SEQ ID NO: 35):

| | |
|---|---|
| atggagacagacacgctccttttgtgggtactgctgctttgggtccctgggtcg | 54 |
| acaggggataagacccacacgtgccctccctgtccagcacccgagttgctcggt | 108 |
| gggccatccgtgtttttgtttcctcccaagcccaaagacacgttgatgattagc | 162 |
| cgcactcccgaggtaacgtgcgtagtggtggatgtgtcacatgaggacccggag | 216 |
| gtgaagttcaattggtacgtggacggagtcgaagtgcacaacgcaaagacgaaa | 270 |
| ccccgagaggaacagtacaactcgacctatcgcgtagtgagcgtactgactgtg | 324 |
| ttgcatcaggattggcttaacggaaaagagtacaagtgtaaagtatccaataag | 378 |
| gccctcccagcgcctattgaaaagacaatcagcaaagcgaaggggcagcctcgc | 432 |
| gaaccgcaagtatataccctcccgcctagccgggacgaattgactaagaatcag | 486 |
| gtcagcctcacatgtctggtcaaaggcttttacccgtcagatatcgcggtcgag | 540 |
| tgggagtccaatgggcagccggaaaacaattacaagacaacgccgccagtcttg | 594 |
| gactcagacgggtcgtttttcctctactcgaaactgacggtggacaagtcccga | 648 |
| tggcagcagggaaatgtattcagctgttcggtcatgcacgaggcgctccacaat | 702 |
| cattatacacaaaagtcgctgtccctgtcgccgggaaagggaggtggcgggtcc | 756 |
| ggcggaggaggatcaggtggtggaggttcagccagaaacggtgatcattgccca | 810 |
| cttggacccgggaggtgctgtcggcttcacactgtcagggcatcactcgaagat | 864 |
| ctcgggtgggcggactgggtgctttcgcccagagaagtgcaagtcactatgtgc | 918 |
| attggtgcgtgcccgtcgcaattcagagctgccaacatgcatgcccagatcaaa | 972 |
| acgagcttgcaccggctgaaacccgacacagtccccgctccgtgctgcgtgccg | 1026 |
| gcgtcgtataaccccatggtcctcatccagaaaaccgatacgggagtgtcattg | 1080 |
| cagacatatgatgaccttttggccaaggattgccactgtatc | 1122 |

Expressed protein (SEQ ID NO: 36):

| | |
|---|---|
| DKTHTCRRCP APELLGGPSV FLFPPKPKDT LMISRTPEVT CVVVDVSHED PEVKFNWYVD | 60 |
| GVEVHNAKTK PREEQYNSTY RVVSVLTVLH QDWLNGKEYK CKVSNKALPA PIEKTISKAK | 120 |
| GQPREPQVYT LPPSRDELTK NQVSLTCLVK GFYPSDIAVE WESNGQPENN YKTTPPVLDS | 130 |
| DGSFFLYSKL TVDKSRWQQG NVFSCSVMHE ALHNHYTQKS LSLSPGKGGG GSGGGGSGGG | 240 |
| GSARNGDHCP LGPGRCCRLH TVRASLEDLG WADWVLSPRE VQVTMCIGAC PSQFPAANMH | 300 |
| AQIKTSLHRL KPDTVPAPCC VPASYNPMVL IQKTDTGVSL QTYDDLLAKD CHCI | 354 |

HSA-hGDF15(197-308), R198H
Open reading frame (SEQ ID NO: 37):

| | |
|---|---|
| atggaaaccgatactctgctgctgtgggtgcttcttctttgggtgccgggatca | 54 |
| accggcgatgcccacaagtcggaggtggcccatcggtttaaggacctcggggag | 108 |
| gagaacttcaaagccctggtcctcatcgccttcgcccaatacctccagcagtgt | 162 |
| ccattcgaagatcacgtgaagctcgtgaacgaagtgactgaatttgccaagact | 216 |
| tgtgtcgcagacgaaagcgccgaaaactgcgacaagtcgttgcatactctcttc | 270 |
| ggggataagctgtgcactgtcgcaacccttagagagacttacggtgaaatggct | 324 |
| gattgctgcgccaaacaagagccggagcgcaacgagtgcttcctccaacataag | 378 |
| gacgacaaccccaacctcccacgcctggtgcggcctgaggtcgacgtcatgtgc | 432 |
| accgctttccatgacaatgaggagacttttctcaagaagtatctgtacgagatc | 486 |
| gcccggaggcacccatacttttatgcaccggagctccttttcttcgctaagcgg | 540 |
| tacaaggcggcgttcactgaatgctgtcaggcagcagacaaggcagcatgcctc | 594 |
| ctgccgaaactggacgaacttcgcgacgagggtaaagcgtcgtccgccaagcag | 648 |
| cgccttaagtgcgcctcgttgcagaagtttggtgaacgcgcattcaaagcgtgg | 702 |
| gccgtcgcaagactttcgcagcggttcccaaaagcggagtttgccgaggtgtcc | 756 |
| aaactggtcaccgacctgaccaaggtccacaccgagtgctgccacggcgatctg | 810 |
| ctcgaatgcgccgacgaccgggctgatctcgcaaagtacatttgcgagaaccaa | 864 |
| gactcgatctcgtcaaaactgaaggaatgctgcgagaagccgctgttggaaaag | 918 |
| agccattgtatcgccgaagtggagaacgatgaaatgcctgctgatctgccaagc | 972 |
| ctcgccgcagactttgtggagagcaaagacgtgtgcaagaactacgccgaagcg | 1026 |
| aaggacgtgtttctcgggatgttcctctacgagtacgcgcgcaggcaccctgac | 1080 |
| tactcagtggtcctgctgttgcggctggccaaaacttacgaaaccaccctcgaa | 1134 |
| aagtgctgcgcggctgccgatccacatgaatgctacgcaaaggtgttcgatgaa | 1188 |
| tttaagcctctggtggaggaaccacagaacctgatcaagcaaaattgtgaactg | 1242 |
| tttgaacagctgggagagtacaaatttcagaatgccctgctggtcagatacact | 1296 |
| aagaaggtgccccaagtctccactccaaccctcgtggaggtgtcacggaatctc | 1350 |
| ggcaaagtgggcagcaaatgctgtaagcacccggaagcaaagaggatgccctgc | 1404 |
| gctgaagattacctgtccgtggtgctgaatcagctttgtgtgctgcacgaaaag | 1458 |
| acgcctgtctccgaccgggtgaccaagtgctgtaccgaatcgctcgtgaatcgc | 1512 |
| agaccctgcttctccgctctcgaagtggacgaaacttacgtcccgaaggagttc | 1566 |
| aatgcggaaaccttcaccttccacgcggacatctgtaccctgagcgaaaaagag | 1620 |
| cggcagatcaagaaacagactgccctggtggaactggtgaagcacaagccgaag | 1674 |
| gcaacgaaggagcagctgaaggcggtgatggatgactttgcagccttcgtggaa | 1728 |
| aagtgttgcaaggcagatgataaagaaacctgtttcgcggaagaggggaagaag | 1782 |
| ttggtggctgccagccaggccgctctcggactgggaggtggaggatcaggaggc | 1836 |
| ggaggctccggaggaggaggctcggctcacaatggcgatcattgcccgctcgga | 1890 |
| ccgggacgctgctgcagactgcataccgtccgcgcttccttggaagatctggga | 1944 |
| tgggcggattgggtgttgtcaccaagagaggtgcaagtgacgatgtgtatcggt | 1998 |
| gcgtgcccttcacagttccgcgctgcgaacatgcatgcccaaatcaagaccagc | 2052 |
| ctgcaccggctgaagccggacactgtcccagctccatgttgcgtgcccgcatcg | 2106 |
| tacaacccgatggtgctcatccagaaaactgacactggagtctcactgcaaacg | 2160 |
| tacgacgatttgctcgccaaagattgccactgcatt | 2196 |

Expressed protein (SEQ ID NO: 38):

| | |
|---|---|
| DAHKSEVAHR FKDLGEENFK ALVLIAFAQY LQQCPFEDHV KLVNEVTEFA KTCVADESAE | 60 |
| NCDKSLHTLF GDKLCTVATL RETYGEMADC CAKQEPERNE CFLQHKDDNP NLPRLVRPEV | 120 |
| DVMCTAFHDN EETFLKKYLY EIARRHPYFY APELLFFAKR YKAAFTECCQ AADKAACLLP | 180 |
| KLDELRDEGK ASSAKQRLKC ASLQKFGERA FKAWAVARLS QRFPKAEFAE VSKLVTDLTK | 240 |
| VHTECCHGDL LECADDRADL AKYICENQDS ISSKLKECCE KPLLEKSHCI AEVENDEMPA | 300 |
| DLPSLAADFV ESKDVCKNYA EAKDVFLGMF LYEYARRHPD YSVVLLLRLA KTYETTLEKS | 360 |
| CAAADPHECY AKVFDEFKPL VEEPQNLIKQ NCELFEQLGE YKFQNALLVR YTKKVPQVST | 420 |
| PTLVEVSRNL GKVGSKCCKH PEAKRMPCAE DYLSVVLNQL CVLHEKTPVS DRVTKCCTES | 480 |
| LVNRRPCFSA LEVDETYVPK EFNAETFTFH ADICTLSEKE RQIKKQTALV ELVKHKPKAT | 540 |
| KEQLKAVMDD FAAFVEKCCK ADDKETCFAE EGKKLVAASQ AALGLGGGGS GGGGSGGGGS | 600 |
| AHNGDHCPLG PGRCCRLHTV RASLEDLGWA DWVLSPREVQ VTMCIGACPS QFRAANMHAQ | 660 |
| IKTSLHRLKP DTVPAPCCVP ASYNPMVLIQ KTDTGVSLQT YDDLLAKDCH CI | 712 |

Open reading frame (SEQ ID NO: 39):

| | |
|---|---|
| atggaaaccgatactcbgctgctgtgggtgcttcttctttgggtgccgggatca | 54 |
| accggcgatgcccacaagtcggaggtggcccatcggtttaaggacctcggggag | 108 |
| gagaacttcaaagccctggtccbcatcgccttcgcccaatacctccagcagtgt | 162 |
| ccattcgaagatcacgcgaagctcgtgaacgaagtgactgaatttgccaagact | 216 |
| tgtgtcgcagacgaaagcgccgaaaactgcgacaagtcgttgcatactctcttc | 270 |
| ggggataagctgtgcactgtcgcaacccttagagagacttacggtgaaatggct | 324 |
| gattgctgcgccaaacaagagccggagcgcaacgagtgcttcctccaacataag | 378 |
| gacgacaaceecaacctcccacgcctggtgcggcctgaggtcgacgtcatgtgc | 432 |
| accgctttccatgacaatgaggagacttttctcaagaagtatctgtacgagatc | 486 |
| gcccggaggcacccatacttttatgcaccggagctccttttcttcgctaagcgg | 540 |
| tacaaggcggegttcaccgaatgctgtcaggcagcagacaaggcagcatgcccc | 594 |
| ctgccgaaactggacgaacttcgcgacgagggtaaagcgtcgtccgccaagcag | 648 |
| cgccttaagtgcgcctcgttgcagaagtttggtgaacgcgcattcaaagcgtgg | 702 |
| gccgtcgcaagactttcgcagcggttcccaaaagcagagttcgccgaggtgtcc | 756 |
| aaactggtcaccgacctgaccaaggtccacaccgagtgctgccacggcgatctg | 810 |
| ctcgaatgcgccgacgaccgggctgatctcgcaaagtacatttgcgagaaccaa | 864 |
| gactcgatctcgtcaaaactgaaggaatgctgcgagaagccgctgttggaaaag | 918 |
| agccactgtatcgccgaagtggagaacgacgaaatgcctgccgatctgccaagc | 972 |
| ctcgccgcagactttgtggagagcaaagacgtgtgcaagaactacgccgaagcg | 1026 |
| aaggacgtgtttctcgggatgttcctctacgagtacgcgcgcaggcaccctgac | 1080 |
| tactcagtggtcctgctgttgcggctggccaaaacttacgaaaccaccctcgaa | 1134 |
| aagtgctgcgcggctgccgatccacatgaatgctacgcaaaggtgttcgatgaa | 1188 |
| tttaagcctctggtggaggaaccacagaacctgatcaagcaaaattgtgaactg | 1242 |
| tttgaacagctgggagagtacaaatttcagaatgccctgctggtcagatacact | 1296 |
| aagaaggtgccccaagtctccactccaaccctcgtggaggtgtcacggaatctc | 1350 |
| ggcaaagtgggcagcaaatgctgtaagcacccggaagcaaagaggatgccctgc | 1404 |
| gctgaagattacctgtccgtggtgctgaatcagctttgtgtgctgcacgaaaag | 1458 |
| acgcctgtctccgaccgggtgaccaagtgctgtaccgaatcgctcgtgaatcgc | 1512 |
| agaccctgcttctccgctctcgaagtggacgaaacttacgtcccgaaggagttc | 1566 |
| aatgcggaaaccttcaccttccacgcggacatctgtaccctgagcgaaaaagag | 1620 |
| cggcagatcaagaaacagactgccctggtggaactggtgaagcacaagccgaag | 1674 |
| gcaacgaaggagcagctgaaggcggtgatggatgactttgcagccttcgtggaa | 1728 |
| aagtgttgcaaggcagatgataaagaaacctgtttcgcggaagaggggaagaag | 1782 |
| ttggtggctgccagccaggccgctctcggactgggaggtggaggatcaggaggc | 1836 |
| ggaggctccggaggaggaggctcggctcacgccggcgatcattgcccgctcgga | 1890 |
| ccgggacgctgctgcagactgcataccgtccgcgcttccttggaagatctggga | 1944 |
| tgggcggattgggtgttgtcaccaagagaggtgcaagtgacgatgtgtatcggt | 1998 |
| gcgtgcccttcacagtcccgcgctgcgaacatgcatgcccaaatcaagaccagc | 2052 |
| ctgcaccggctgaagccggacactgtcccagctccatgttgcgtgcccgcatcg | 2106 |
| tacaacccgatggtgctcatccagaaaactgacactggagtctcactgcaaacg | 2160 |
| tacgacgatttgctcgceaaagattgccactgcatt | 2196 |

Expressed protein (SEQ ID NO: 40):

| | |
|---|---|
| DAHKSEVAHR FKDLGEENFK ALVLIAFAQY LQQCPFEDHV KLVNEVTEFA KTCVADESAE | 60 |
| NCDKSLHTLF GDKLCTVATL RETYGEMADC CAKQEPERNE CFLQHKDDNP NLPRLVRPEV | 120 |
| DVMCTAFHDN EETFLKKYLY EIARRHPYFY APELLFFAKR YKAAFTECCQ AADKAACLLP | 180 |
| KLDELRDEGK ASSAKQPLKC ASLQKFGERA FKAWAVARLS QRFPKAEFAE VSKLVTDLTK | 240 |
| VHTECCHGDL LECADDRADL AKYICENQDS ISSKLKECCE KPLLEKSHCI AEVENDEMPA | 300 |
| DLPSLAADFV ESKDVCKNYA EAKDVFLGMF LYEYARRHPD YSVVLLLRLA KTYETTLEKC | 360 |
| CAAADPHECY AKVFDEFKPL VEEPQNLIKQ NCELFEQLGE YKFQNALLVR YTKKVPQVST | 420 |
| PTLVEVSRNL GKVGSKCCKH PEAKRMPCAE DYLSVVLNQL CVLHEKTPVS DRVTKCCTES | 480 |
| LVNRRPCFSA LEVDETYVPK EFNAETFTFH ADICTLSEKE RQIKKQTALV ELVKHKPKAT | 540 |
| KEQLKAVMDD FAAFVEKCCK ADDKETCFAE EGKKLVAASQ AALGLGGGGS GGGGSGGGGS | 600 |
| AHAGDHCPLG PGRCCRLHTV RASLEDLGWA DWVLSPREVQ VTMCIGACPS QFRAANMHAQ | 660 |
| IKTSLHRLKP DTVPAPCCVP ASYNPMVLIQ KTDTGVSLQT YDDLLAKDCH CI | 712 |

HSA-hGDF15(197-308), N199E
Open reading frame (SEQ ID NO: 41):

| | |
|---|---|
| atggaaaccgatactctgctgctgtgggtgcttcttctttgggtgccgggatca | 54 |
| accggcgatgcccacaagtcggaggtggcccatcggtttaaggacctcggggag | 108 |
| gagaacttcaaagccctggtcctcatcgccttcgcccaatacctccagcagtgt | 162 |
| ccattcgaagatcacgtgaagctcgtgaacgaagtgactgaatttgccaagact | 216 |
| tgtgtcgcagacgaaagcgccgaaaactgcgacaagtcgttgcatactctcttc | 270 |
| ggggataagctgtgcactgtcgcaacccttagagagacttacggtgaaatggct | 324 |
| gattgctgcgccaaacaagagccggagcgcaacgagtgcttcctccaacataag | 378 |
| gacgacaaccccaacctcccacgcctggtgcggcctgaggtcgacgtcatgtgc | 432 |
| accgctttccatgacaatgaggagacttttctcaagaagtatctgtacgagatc | 486 |
| gcccggaggcacccatacttttatgcaccggagctccttttcttcgctaagcgg | 540 |
| tacaaggcggcgttcactgaatgctgtcaggcagcagacaaggcagcatgcctc | 594 |
| ctgccgaaactggacgaacttcgcgacgagggtaaagcgtcgtccgccaagcag | 648 |
| cgccttaagtgcgcctcgttgcagaagtttggtgaacgcgcattcaaagcgtgg | 702 |
| gccgtcgcaagactttcgcagcggttcccaaaagcggagtttgccgaggtgtcc | 756 |
| aaactggtcaccgacctgaccaaggtccacaccgagtgctgccacggcgatctg | 810 |
| ctcgaatgcgccgacgaccgggctgatctcgcaaagtacatttgcgagaaccaa | 864 |
| gactcgatctcgtcaaaactgaaggaatgctgcgagaagccgctgttggaaaag | 918 |
| agccattgtatcgccgaagtggagaacgatgaaatgcctgctgatctgccaagc | 972 |
| ctcgccgcagactttgtggagagcaaagacgtgtgcaagaactacgccgaagcg | 1026 |
| aaggacgtgtttctcgggatgttcctctacgagtacgcgcgcaggcaccctgac | 1080 |
| tactcagtggtcctgctgttgcggctggccaaaacttacgaaaccaccctcgaa | 1134 |
| aagtgctgcgcggctgccgatccacatgaatgctacgcaaaggtgttcgatgaa | 1188 |
| tttaagcctctggtggaggaaccacagaacctgatcaagcaaaattgtgaactg | 1242 |
| tttgaacagctgggagagtacaaatttcagaatgccctgctggtcagatacact | 1296 |
| aagaaggtgccccaagtctccactccaaccctcgtggaggtgtcacggaatctc | 1350 |
| ggcaaagtgggcagcaaatgctgtaagcacccggaagcaaagaggatgccctgc | 1404 |
| gctgaagattacctgtccgtggtgctgaatcagctttgtgtgctgcacgaaaag | 1458 |
| acgcctgtctccgaccgggtgaccaagtgctgtaccgaatcgctcgtgaatcgc | 1512 |
| agaccctgcttctccgctctcgaagtggacgaaacttacgtcccgaaggagttc | 1566 |
| aatgcggaaaccttcaccttccacgcggacatctgtaccctgagcgaaaaagag | 1620 |
| cggcagatcaagaaacagactgccctggtggaactggtgaagcacaagccgaag | 1674 |
| gcaacgaaggagcagctgaaggcggtgatggatgactttgcagccttcgtggaa | 1728 |
| aagtgttgcaaggcagatgataaagaaacctgtttcgcggaagaggggaagaag | 1782 |
| ttggtggctgccagccaggccgctctcggactgggaggtggaggatcaggaggc | 1836 |
| ggaggctccggaggaggaggctcggctcgcgagggcgatcattgcccgctcgga | 1890 |
| ccgggacgctgctgcagactgcataccgtccgcgcttccttggaagatctggga | 1944 |
| tgggcggattgggtgttgtcaccaagagaggtgcaagtgacgatgtgtatcggt | 1998 |
| gcgtgcccttcacagttccgcgctgcgaacatgcatgcccaaatcaagaccagc | 2052 |
| ctgcaccggctgaagccggacactgtcccagctccatgttgcgtgcccgcatcg | 2106 |
| tacaacccgatggtgctcatccagaaaactgacactggagtctcactgcaaacg | 2160 |
| tacgacgatttgctcgccaaagattgccactgcatt | 2196 |

Expressed protein (SEQ ID NO: 42):

| | |
|---|---|
| DAHKSEVAHR FKDDGEENFK ALVLIAFAQY LQQCPFEDHV KLVNEVTEFA KTCVADESAE | 60 |
| NCDKSLHTLF GDKLCTVATL RETYGEMADC CAKQEPERNE CFLQHKDDNP NLPRLVRPEV | 120 |
| DVMCTAFHDN EETELKKYLY EIARRHPYFY APELLFFAKR YKAAFTECCQ AADKAACLLP | 180 |
| KLDELRDEGK ASSAKQRLKC ASLQKFGERA FKAWAVARLS QRFPKAFFAE VSKLVTDLTK | 240 |
| VHTECCHGDL LECADDRADL AKYICENQDS ISSKLKECCE KPLLEKSHCI AEVENDEMPA | 300 |
| DLPSLAADFV ESKDVCKNYA EAKDVFLGMF LYEYARRHPD YSVVLLLRLA KTYETTLEKC | 360 |
| CAAADPHECY AKVFDEFKPL VEEPQNLIKQ NCELFEQLGE YKFQNALLVR YTKKVPQVST | 420 |
| PTLVEVSRNL GKVGSKCCKH PEAKRMPCAE DYLSVVLNQL CVLHEKTPVS DPVTKCCTES | 480 |
| LVNRRPCFSA LEVDETYVPK EFNAETFTFH ADICTLSEKE RQIKKQTALV ELVKHKPKAT | 540 |
| KEQLKAVMDD FAAFVEKCCK ADDKETCFAE EGKKLVAASQ AALGLGGGGS GGGGSGGGGS | 600 |
| AREGDHCPLG PGRCCRLHTV RASLEDLGWA DWVLSPREVQ VTMCIGACPS QFRAANMHAQ | 660 |
| IKTSLHRLKP DTVPAPCCVP ASYNPMVLIQ KTDTGVSLQT YDDLLAKDCH CI | 712 |

Mature human GDF15(197-308) (SEQ ID NO: 44)
ARNG DHCPLGPGRC CRLHTVRASL EDLGWADWVL SPREVQVTMC IGACPSQFRA
ANMHAQIKTS LHRLKPDTVP APCCVPASYN PMVLIQKTDT GVSLQTYDDL
LAKDCHCI
Mature human SA (25-609) (SEQ ID NO: 45)
DAHKSE VAHRFKDLGE ENFKALVLIA FAQYLQQCPF
EDHVKLVNEV TEFAKTCVAD ESAENCDKSL HTLFGDKLCT VATLRETYGE MADCCAKQEP
ERNECFLQHK DDNPNLPRLV RPEVDVMCTA FHDNEETFLK KYLYEIARRH PYFYAPELLF
FAKRYKAAFT ECCQAADKAA CLLPKLDELR DEGKASSAKQ RLKCASLQKF GERAFKAWAV
ARLSQRFPKA EFAEVSKLVT DLTKVHTECC HGDLLECADD RADLAKYICE NQDSISSKLK
ECCEKPLLEK SHCIAEVEND EMPADLPSLA ADFVESKDVC KNYAEAKDVF LGMFLYEYAR
RHPDYSVVLL LRLAKTYETT LEKCCAAADP HECYAKVFDE FKPLVEEPQN LIKQNCELFE
QLGEYKFQNA LLVRYTKKVP QVSTPTLVEV SRNLGKVGSK CCKHPEAKRM PCAEDYLSVV
LNQLCVLHEK TPVSDRVTKC CTESLVNRRP CFSALEVDET YVPKEFNAET FTFHADICTL
SEKERQIKKQ TALVELVKHK PKATKEQLKA VMDDFAAFVE KCCKADDKET CFAEEGKKLV
AASQAALGL
MSA-(4GS)₃-GDF15(197-308)(Q247R)
DNA (SEQ ID NO: 65)
Atggagactgataccctgctcctctgggtgctgcttctctgggtccctggctcaaccggcga
agcccacaagtccgagatcgcccatcgctataatgctcttggagaacagcatttcaagggac
tggtgctgattgccttctcccagtacctccaaaaggccagctatgatgagcacgccaagctc
gtccaagaagtcaccgactttgctaagacttgtgtggccgacgaaagcgctgccaattgcga
taagtcactccatactctcttcggggacaagctgtgcgctattcccaacctccgcgagaatt
acggtgagctggccgactgttgcaccaaacaggagccagagcggaacgagtgcttccttcaa
cacaaagatgacaatccttcactgcctcctttcgaacggcccgaggcagaggcaatgtgcac
tagcttcaaggagaacccaaccaccttcatgggacactacctccatgaggtcgctagacggc
atccctacttctatgccccagagcttctgtattatgcagaacagtacaatgagatcctgacc
cagtgctgtgctgaggctgataaggagagctgcctgaccccaaagctcgacggagtgaagga
aaaggctcttgtgtccagcgtgcggcagcgcatgaagtgctcttcaatgcagaagtttgggg
agcgcgccttcaaagcctgggccgtggccagactgtcccagacctttcctaatgctgacttt
gccgagatcaccaagctcgctactgacctgaccaaggtcaacaaagagtgttgccacggaga
tctgctcgaatgcgccgacgaccgcgctgagcttgctaagtacatgtgcgaaaaccaggcaa
ccatttctagcaagctgcagacctgttgtgataagcctctgctgaagaaagcccattgcctc
agcgaggtcgaacatgacactatgccggcagacctccccgctatcgccgctgacttcgtgga
ggaccaagaagtgtgcaagaattacgccgaggctaaggacgtgttccttggtactttcctct
acgagtatagccggaggcaccctgactacagcgtgtctcttctgcttcggctcgccaagaag
tacgaagccaccctcgaaaaatgctgcgccgaagcaaatccgccagcttgttacgggactgt
gctggctgagtttcagcccctggtggaagagcccaagaacctcgtcaagaccaactgcgacc
tttacgagaaactgggtgaatacgggtttcagaatgccattctggtgcggtacacccagaag
gcaccacaagtgtccaccccaacccttgtcgaggcagcccgcaaccttggacgcgtcgggac
caagtgttgtaccctgcccgaggaccaacgcctgccctgcgtcgaggactaccttagcgcca
ttctgaacagagtctgtctgctccatgaaaagacccctgtgtctgagcacgtgaccaagtgc
tgttcaggctcactggtggagaggaggccttgcttttctgccctgaccgtggacgaaaccta
cgtgcccaaggagttcaaagctgaaaccttcactttccattcagacatctgtaccctccccg
aaaaggaaaagcaaatcaagaagcagaccgcccttgctgaactggtgaagcacaagccaaag
gccaccgccgaacaactcaagactgtgatggacgacttcgctcagttcctcgacacttgctg
caaagccgccgacaaagatacctgtttctcaaccgaggggccgaacctggtgactagagcca
aggacgccctggccggaggaggtggttctggcggtggtggttccggcggaggaggatctgcc
aggaatggagatcactgcccactcggaccgggacggtgttgtcgcctgcacactgtgcgcgc
atctcttgaggatctgggatgggctgattgggtgctctctcccagagaggtgcaagtcacca
tgtgcattggcgcctgcccctccaggttcagggcagctaacatgcatgctcagatcaagact
agcctgcacaggctgaagcccgacactgtccctgccccatgttgtgtgccggcctcctataa
cccaatggtcctgatccaaaagaccgataccggagtgtcacttcagacttacgacgatctgc
ttgcaaaagactgccattgcatctga
Protein (SEQ ID NO: 66)
eahkseiahrynalgeqhfkglvliafsqylqkasydehaklvqevtdfaktcvadesaanc
dkslhtlfgdklcaipnlrenygeladcctkqepernecflqhkddnpslppferpeaeamc
tsfkenpttfmghylhevarrhpyfyapellyyaegyneiltqccaeadkescltpkldgvk
ekalvssvrqrmkcssmqkfgerafkawavarlsqtfpnadfaeitklatdltkvnkecchg
dllecaddraelakymcenqatissklqtccdkpllkkahclsevehdtmpadlpaiaadfv
edgevcknyaeakdvflgtflyeysrrhpdysyslllrlakkyeatlekccaeanppacygt
vlaefqplveepknlvktncdlyeklgeygfqnailvrytqkapqvstptiveaarnlgrvg
tkcctlpedqrlpcvedylsailnrvcllhektpvsehvtkccsgslverrpcfsaltvdet
yvpkefkaetftfhsdictlpekekqikkqtalaelvkhkpkataeqlktvmddfaqfldtc
ckaadkdtcfstegpnlvtrakdalaggggsggggsggggsarngdhcplgpgrccrlhtvr
asledlgwadwvlsprevqvtmcigacpsrfraanmhaqiktslhrlkpdtvpapccvpasy
npmvliqktdtgvslqtyddllakdchci
MSA-(4GS)₃-GDF15(197-308)(S278R)
DNA (SEQ ID NO: 67)
atggagactgataccctgctcctctgggtgctgcttctctgggtccctggctcaaccggcga
agcccacaagtccgagatcgcccatcgctataatgctcttggagaacagcatttcaagggac
tggtgctgattgccttctcccagtacctccaaaaggccagctatgatgagcacgccaagctc
gtccaagaagtcaccgactttgctaagacttgtgtggccgacgaaagcgctgccaattgcga
taagtcactccatactctcttcggggacaagctgtgcgctattcccaacctccgcgagaatt
acggtgagctggccgactgttgcaccaaacaggagccagagcggaacgagtgcttccttcaa
cacaaagatgacaatccttcactgcctcctttcgaacggcccgaggcagaggcaatgtgcac
tagcttcaaggagaacccaaccaccttcatgggacactacctccatgaggtcgctagacggc
atccctacttctatgccccagagcttctgtattatgcagaacagtacaatgagatcctgacc
cagtgctgtgctgaggctgataaggagagctgcctgaccccaaagctcgacggagtgaagga
aaaggctcttgtgtccagcgtgcggcagcgcatgaagtgctcttcaatgcagaagtttgggg
agcgcgccttcaaagcctgggccgtggccagactgtcccagacctttcctaatgctgacttt
gccgagatcaccaagctcgctactgacctgaccaaggtcaacaaagagtgttgccacggaga
tctgctcgaatgcgccgacgaccgcgctgagcttgctaagtacatgtgcgaaaaccaggcaa
ccatttctagcaagctgcagacctgttgtgataagcctctgctgaagaaagcccattgcctc
agcgaggtcgaacatgacactatgccggcagacctccccgctatcgccgctgacttcgtgga
ggaccaagaagtgtgcaagaattacgccgaggctaaggacgtgttccttggtactttcctct
acgagtatagccggaggcaccctgactacagcgtgtctcttctgcttcggctcgccaagaag
tacgaagccaccctcgaaaaatgctgcgccgaagcaaatccgccagcttgttacgggactgt
gctggctgagtttcagcccctggtggaagagcccaagaacctcgtcaagaccaactgcgacc
tttacgagaaactgggtgaatacgggtttcagaatgccattctggtgcggtacacccagaag
gcaccacaagtgtccaccccaacccttgtcgaggcagcccgcaaccttggacgcgtcgggac
caagtgttgtaccctgcccgaggaccaacgcctgccctgcgtcgaggactaccttagcgcca
ttctgaacagagtctgtctgctccatgaaaagacccctgtgtctgagcacgtgaccaagtgc
tgttcaggctcactggtggagaggaggccttgcttttctgccctgaccgtggacgaaaccta
cgtgcccaaggagttcaaagctgaaaccttcactttccattcagacatctgtaccctccccg
aaaaggaaaagcaaatcaagaagcagaccgcccttgctgaactggtgaagcacaagccaaag
gccaccgccgaacaactcaagactgtgatggacgacttcgctcagttcctcgacacttgctg
caaagccgccgacaaagatacctgtttctcaaccgaggggccgaacctggtgactagagcca
aggacgccctggccggaggaggtggttctggcggtggtggttccggcggaggaggatctgcc
aggaatggagatcactgcccactcggaccgggacggtgttgtcgcctgcacactgtgcgcgc
atctcttgaggatctgggatgggctgattgggtgctctctcccagagaggtgcaagtcacca
tgtgcattggcgcctgcccctcccaattcagggcagctaacatgcatgctcagatcaagact
agcctgcacaggctgaagcccgacactgtccctgccccatgttgtgtgccggccaggtataa
cccaatggtcctgatccaaaagaccgataccggagtgtcacttcagacttacgacgatctgc
ttgcaaaagactgccattgcatctga
Protein (SEQ ID NO: 68)
eahkseiahrynalgeqhfkglvliafsqylqkasydehaklvqevtdfaktcvadesaanc
dkslhtlfgdklcaipnlrenygeladcctkqepernecflqhkddnpslppferpeaeamc
tsfkenpttfmghylhevarrhpyfyapellyyaegyneiltqccaeadkescltpkldgvk
ekalvssvrqrmkcssmqkfgerafkawavarlsqtfpnadfaeitklatdltkvnkecchg
dllecaddraelakymcenqatissklqtccdkpllkkahclsevehdtmpadlpaiaadfv
edgevcknyaeakdvflgtflyeysrrhpdysyslllrlakkyeatlekccaeanppacygt
vlaefqplveepknlvktncdlyeklgeygfqnailvrytqkapqvstptiveaarnlgrvg
tkcctlpedqrlpcvedylsailnrvcllhektpvsehvtkccsgslverrpcfsaltvdet
yvpkefkaetftfhsdictlpekekqikkqtalaelvkhkpkataeqlktvmddfaqfldtc
ckaadkdtcfstegpnlvtrakdalaggggsggggsggggsarngdhcplgpgrccrlhtvr
asledlgwadwvlsprevqvtmcigacpsqfraanmhaqiktslhrlkpdtvpapccvpary
npmvliqktdtgvslqtyddllakdchci
MSA-(4GS)₃-GDF15(197-308)(D289R)
DNA (SEQ ID NO: 69)
Atggagactgataccctgctcctctgggtgctgcttctctgggtccctggctcaaccggcga
agcccacaagtccgagatcgcccatcgctataatgctcttggagaacagcatttcaagggac
tggtgctgattgccttctcccagtacctccaaaaggccagctatgatgagcacgccaagctc
gtccaagaagtcaccgactttgctaagacttgtgtggccgacgaaagcgctgccaattgcga
taagtcactccatactctcttcggggacaagctgtgcgctattcccaacctccgcgagaatt
acggtgagctggccgactgttgcaccaaacaggagccagagcggaacgagtgcttccttcaa
cacaaagatgacaatccttcactgcctcctttcgaacggcccgaggcagaggcaatgtgcac
tagcttcaaggagaacccaaccaccttcatgggacactacctccatgaggtcgctagacggc
atccctacttctatgccccagagcttctgtattatgcagaacagtacaatgagatcctgacc
cagtgctgtgctgaggctgataaggagagctgcctgaccccaaagctcgacggagtgaagga
aaaggctcttgtgtccagcgtgcggcagcgcatgaagtgctcttcaatgcagaagtttgggg
agcgcgccttcaaagcctgggccgtggccagactgtcccagacctttcctaatgctgacttt
gccgagatcaccaagctcgctactgacctgaccaaggtcaacaaagagtgttgccacggaga
tctgctcgaatgcgccgacgaccgcgctgagcttgctaagtacatgtgcgaaaaccaggcaa
ccatttctagcaagctgcagacctgttgtgataagcctctgctgaagaaagcccattgcctc
agcgaggtcgaacatgacactatgccggcagacctccccgctatcgccgctgacttcgtgga
ggaccaagaagtgtgcaagaattacgccgaggctaaggacgtgttccttggtactttcctct
acgagtatagccggaggcaccctgactacagcgtgtctcttctgcttcggctcgccaagaag
tacgaagccaccctcgaaaaatgctgcgccgaagcaaatccgccagcttgttacgggactgt
gctggctgagtttcagcccctggtggaagagcccaagaacctcgtcaagaccaactgcgacc
tttacgagaaactgggtgaatacgggtttcagaatgccattctggtgcggtacacccagaag
gcaccacaagtgtccaccccaacccttgtcgaggcagcccgcaaccttggacgcgtcgggac
caagtgttgtaccctgcccgaggaccaacgcctgccctgcgtcgaggactaccttagcgcca
ttctgaacagagtctgtctgctccatgaaaagacccctgtgtctgagcacgtgaccaagtgc
tgttcaggctcactggtggagaggaggccttgcttttctgccctgaccgtggacgaaaccta
cgtgcccaaggagttcaaagctgaaaccttcactttccattcagacatctgtaccctccccg
aaaaggaaaagcaaatcaagaagcagaccgcccttgctgaactggtgaagcacaagccaaag
gccaccgccgaacaactcaagactgtgatggacgacttcgctcagttcctcgacacttgctg
caaagccgccgacaaagatacctgtttctcaaccgaggggccgaacctggtgactagagcca
aggacgccctggccggaggaggtggttctggcggtggtggttccggcggaggaggatctgcc
aggaatggagatcactgcccactcggaccgggacggtgttgtcgcctgcacactgtgcgcgc
atctcttgaggatctgggatgggctgattgggtgctctctcccagagaggtgcaagtcacca
tgtgcattggcgcctgcccctcccaattcagggcagctaacatgcatgctcagatcaagact
agcctgcacaggctgaagcccgacactgtccctgccccatgttgtgtgccggcctcctataa
cccaatggtcctgatccaaaagaccaggaccggagtgtcacttcagacttacgacgatctgc
ttgcaaaagactgccattgcatctga
Protein (SEQ ID NO: 70)
Eahkseiahrynalgeqhfkglvliafsqylqkasydehaklvqevtdfaktcvadesaanc
dkslhtlfgdklcaipnlrenygeladcctkqepernecflqhkddnpslppferpeaeamc
tsfkenpttfmghylhevarrhpyfyapellyyaegyneiltqccaeadkescltpkldgvk
ekalvssvrqrmkcssmqkfgerafkawavarlsqtfpnadfaeitklatdltkvnkecchg
dllecaddraelakymcenqatissklqtccdkpllkkahclsevehdtmpadlpaiaadfv
edgevcknyaeakdvflgtflyeysrrhpdysyslllrlakkyeatlekccaeanppacygt
vlaefqplveepknlvktncdlyeklgeygfqnailvrytqkapqvstptiveaarnlgrvg
tkcctlpedqrlpcvedylsailnrvcllhektpvsehvtkccsgslverrpcfsaltvdet
yvpkefkaetftfhsdictlpekekqikkqtalaelvkhkpkataeqlktvmddfaqfldtc
ckaadkdtcfstegpnlvtrakdalaggggsggggsggggsarngdhcplgpgrccrlhtvr
asledlgwadwvlsprevqvtmcigacpsqfraanmhaqiktslhrlkpdtvpapccvpasy
npmvliqktrtgvslqtyddllakdchci
MSA-(4GS)3-GDF15(197-308)(L294R)
DNA (SEQ ID NO: 71)
atggagactgataccctgctcctctgggtgctgcttctctgggtccctggctcaaccggcga
agcccacaagtccgagatcgcccatcgctataatgctcttggagaacagcatttcaagggac
tggtgctgattgccttctcccagtacctccaaaaggccagctatgatgagcacgccaagctc
gtccaagaagtcaccgactttgctaagacttgtgtggccgacgaaagcgctgccaattgcga
taagtcactccatactctcttcggggacaagctgtgcgctattcccaacctccgcgagaatt
acggtgagctggccgactgttgcaccaaacaggagccagagcggaacgagtgcttccttcaa
cacaaagatgacaatccttcactgcctcctttcgaacggcccgaggcagaggcaatgtgcac
tagcttcaaggagaacccaaccaccttcatgggacactacctccatgaggtcgctagacggc
atccctacttctatgccccagagcttctgtattatgcagaacagtacaatgagatcctgacc
cagtgctgtgctgaggctgataaggagagctgcctgaccccaaagctcgacggagtgaagga
aaaggctcttgtgtccagcgtgcggcagcgcatgaagtgctcttcaatgcagaagtttgggg
agcgcgccttcaaagcctgggccgtggccagactgtcccagacctttcctaatgctgacttt
gccgagatcaccaagctcgctactgacctgaccaaggtcaacaaagagtgttgccacggaga
tctgctcgaatgcgccgacgaccgcgctgagcttgctaagtacatgtgcgaaaaccaggcaa
ccatttctagcaagctgcagacctgttgtgataagcctctgctgaagaaagcccattgcctc
agcgaggtcgaacatgacactatgccggcagacctccccgctatcgccgctgacttcgtgga
ggaccaagaagtgtgcaagaattacgccgaggctaaggacgtgttccttggtactttcctct
acgagtatagccggaggcaccctgactacagcgtgtctcttctgcttcggctcgccaagaag
tacgaagccaccctcgaaaaatgctgcgccgaagcaaatccgccagcttgttacgggactgt
gctggctgagtttcagcccctggtggaagagcccaagaacctcgtcaagaccaactgcgacc
tttacgagaaactgggtgaatacgggtttcagaatgccattctggtgcggtacacccagaag
gcaccacaagtgtccaccccaacccttgtcgaggcagcccgcaaccttggacgcgtcgggac
caagtgttgtaccctgcccgaggaccaacgcctgccctgcgtcgaggactaccttagcgcca
ttctgaacagagtctgtctgctccatgaaaagacccctgtgtctgagcacgtgaccaagtgc
tgttcaggctcactggtggagaggaggccttgcttttctgccctgaccgtggacgaaaccta
cgtgcccaaggagttcaaagctgaaaccttcactttccattcagacatctgtaccctccccg
aaaaggaaaagcaaatcaagaagcagaccgcccttgctgaactggtgaagcacaagccaaag
gccaccgccgaacaactcaagactgtgatggacgacttcgctcagttcctcgacacttgctg
caaagccgccgacaaagatacctgtttctcaaccgaggggccgaacctggtgactagagcca
aggacgccctggccggaggaggtggttctggcggtggtggttccggcggaggaggatctgcc
aggaatggagatcactgcccactcggaccgggacggtgttgtcgcctgcacactgtgcgcgc
atctcttgaggatctgggatgggctgattgggtgctctctcccagagaggtgcaagtcacca
tgtgcattggcgcctgcccctcccaattcagggcagctaacatgcatgctcagatcaagact
agcctgcacaggctgaagcccgacactgtccctgccccatgttgtgtgccggcctcctataa
cccaatggtcctgatccaaaagaccgataccggagtgtcaaggcagacttacgacgatctgc
ttgcaaaagactgccattgcatctga
Protein (SEQ ID NO: 72)
Eahkseiahrynalgeqhfkglvliafsqylqkasydehaklvqevtdfaktcvadesaanc
dkslhtlfgdklcaipnlrenygeladcctkqepernecflqhkddnpslppferpeaeamc
tsfkenpttfmghylhevarrhpyfyapellyyaegyneiltqccaeadkescltpkldgvk
ekalvssvrqrmkcssmqkfgerafkawavarlsqtfpnadfaeitklatdltkvnkecchg
dllecaddraelakymcenqatissklqtccdkpllkkahclsevehdtmpadlpaiaadfv
edgevcknyaeakdvflgtflyeysrrhpdysyslllrlakkyeatlekccaeanppacygt
vlaefqplveepknlvktncdlyeklgeygfqnailvrytqkapqvstptiveaarnlgrvg
tkcctlpedqrlpcvedylsailnrvcllhektpvsehvtkccsgslverrpcfsaltvdet
yvpkefkaetftfhsdictlpekekqikkqtalaelvkhkpkataeqlktvmddfaqfldtc
ckaadkdtcfstegpnlvtrakdalaggggsggggsggggsarngdhcplgpgrccrlhtvr
asledlgwadwvlsprevqvtmcigacpsqfraanmhaqiktslhrlkpdtvpapccvpasy
npmvliqktdtgvsrqtyddllakdchci

1. A fusion polypeptide comprising a) first moiety and b) second moiety, wherein
the first moiety is human serum albumin or a functional variant thereof;
the second moiety is human Grown Differentiation Factor 15 (GDF15) protein or a functional variant thereof; and
the first moiety is amino terminal to the second moiety.

2. The fusion polypeptide of claim 1, further comprising a linker that links the first moiety to the second moiety.

3. The fusion polypeptide of claim 1, wherein the first moiety has at least 80% sequence identity to SEQ ID NO:45.

4. (canceled)

5. The fusion polypeptide of claim 1, wherein the second moiety has at least 80% sequence identity to SEQ ID NO:44.

6. (canceled)

7. The fusion polypeptide of claim 1 wherein:
the first moiety is selected from the group consisting of HSA (25-609) (SEQ ID NO:45), and HSA(25-609) in which Cys34 is replaced with Ser and Asn503 is replaced with Gln; and
the second moiety is selected from the group consisting human mature GDF15 peptide (197-308) (SEQ ID NO:44), human GDF15(211-308) (amino acids 211-308 of SEQ ID NO:1), human GDF15(197-308) (SEQ ID NO:44) in which Cys203 is replaced with Ser (C203S) and Cys210 is replaced with Ser (C210S), human GDF15(197-308) (SEQ ID NO:44) in which Cys273 is replaced with Ser (C273S).

8. The fusion polypeptide of claim 1, wherein
a) the amino acid residue in the GDF15 protein or a functional variant thereof that corresponds to position 198 of SEQ ID NO:1 is not Arg;
b) the amino acid residue in the GDF15 protein or a functional variant thereof that corresponds to position 199 of SEQ ID NO:1 is not Asn; or
c) the amino acid residue in the GDF15 protein or a functional variant thereof that corresponds to position 198 of SEQ ID NO:1 is not Arg and the amino acid residue in the GDF15 protein or a functional variant thereof that corresponds to position 199 of SEQ ID NO:1 is not Asn.

9. The fusion polypeptide of claim 8, wherein amino acid position 198 is His and amino acid position 199 is Ala.

10. The fusion polypeptide of claim 1, wherein the GDF15 protein or a functional variant thereof further comprises an amino acid replacement or deletion of one or more surface exposed residues, one or more N-terminal amino acids (amino acids 197-210), Cys 203, Cys 210 and/or Cys273.

11. The fusion polypeptide of claim 10, wherein one or more of the surface exposed residues are selected from a group consisting of Arg217, Ser219, Ala226, Glu234, Ala243, Ser246, Gln247, Arg263, Lys265, Thr268, Ala277, Asn280, Lys287, Thr290, Lys303 and Asp304.

12. The fusion polypeptide of claim 2, wherein the linker comprises the amino acid sequence selected from the group consisting of (GGGGSer)n and (GPPGS)n, wherein n is one to about 20.

13. The fusion polypeptide of claim 12, wherein the linker is (GGGGS)n, and n is 3.

14. The fusion polypeptide of claim 1, wherein the fusion polypeptide comprises an amino acid sequence selected from the group consisting of SEQ ID NOs: 20, 26, 28, 30, 32, 38, 40 and 42.

15. (canceled)

16. A pharmaceutical composition comprising the fusion polypeptide of claim 1 and a pharmaceutically or physiologically acceptable carrier.

17. (canceled)

18. A method for decreasing appetite and/or body weight in a subject, comprising administering to a subject in need thereof an effective amount of the fusion polypeptide of claim 1.

19. A method of treating a metabolic disease in a subject, comprising administering to a subject in need thereof a therapeutically effective amount of the fusion polypeptide of claim 1.

20. The method of claim 18, wherein the subject is overweight or obese.

21. An isolated nucleic acid molecule encoding the fusion polypeptide of claim 1.

22. A host cell comprising a recombinant nucleic acid that encodes the fusion polypeptide of claim 1.

23. A method for making a fusion polypeptide comprising maintaining the host cell of claim 22 under conditions suitable for expression of the recombinant nucleic acid, whereby the recombinant nucleic acid is expressed and the fusion polypeptide is produced.

24. (canceled)

25. The method of claim 19, wherein the metabolic disease is selected from the group consisting of obesity, type 2 diabetes mellitus, pancreatitis, dyslipidemia, nonalcoholic steatohepatitis (NASH), insulin resistance, hyperinsulinemia, glucose intolerance, hyperglycemia, metabolic syndrome, hypertension, cardiovascular disease, atherosclerosis, peripheral arterial disease, stroke, heart failure, coronary heart disease, diabetic complications (including but not limited to chronic kidney disease), neuropathy, and gastroparesis.


SEQUENCE LISTING (78 sequences)

SEQ ID NO: 1 - protein, 308 aa, Homo sapiens
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 2 - protein, 609 aa, Homo sapiens [sequence omitted]

SEQ ID NO: 3 - protein, 20 aa, Mus musculus
METDTLLLWVLLLWVPGSTG

SEQ ID NO: 4 - protein, 21 aa, Homo sapiens
MALPVTALLLPLALLLHAARP

SEQ ID NO: 5 - protein, 85 aa, Artificial Sequence
  source: /note=“Description of Artificial Sequence Synthetic polypeptide”
MRFPSIFTAVLFAASSALAAPANTTTEDETAQIPAEAVIDYSDLEGDFDAAALPLSNSTNNGLSSTNTTIASIAAKEEGVQLDKR

SEQ ID NO: 6 - DNA, 414 bp, Artificial Sequence
  source: /note=“Description of Artificial Sequence Synthetic polynucleotide”
atggagacagacacgctgctcctctgggtattgctgctgtgggtaccaggatccaccggccatcaccaccaccatcatgccagaaacggtgatcattgcccacttggacccgggaggtgctgtcggcttcacactgtcagggcatcactcgaagatctcgggtgggcggactgggtgctttcgcccagagaagtgcaagtcactatgtgcattggtgcgtgcccgtcgcaattcagagctgccaacatgcatgcccagatcaaaacgagcttgcaccggctgaaacccgacacagtccccgctccgtgctgcgtgccggcgtcgtataaccccatggtcctcatccagaaaaccgatacgggagtgtcattgcagacatatgatgaccttttggccaaggattgccactgtatc

SEQ ID NO: 7 - protein, 118 aa, Artificial Sequence
  source: /note=“Description of Artificial Sequence Synthetic polypeptide”
HHHHHHARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 8 - DNA, 450 bp, Artificial Sequence
  source: /note=“Description of Artificial Sequence Synthetic polynucleotide”
atggagacagacacgctgctcctctgggtattgctgctgtgggtaccaggatccaccggccatcaccaccaccatcatcaccacggcggaagcgagaacctgtacttccagggcgccagaaacggtgatcattgcccacttggacccgggaggtgctgtcggcttcacactgtcagggcatcactcgaagatctcgggtgggcggactgggtgctttcgcccagagaagtgcaagtcactatgtgcattggtgcgtgcccgtcgcaattcagagctgccaacatgcatgcccagatcaaaacgagcttgcaccggctgaaacccgacacagtccccgctccgtgctgcgtgccggcgtcgtataaccccatggtcctcatccagaaaaccgatacgggagtgtcattgcagacatatgatgaccttttggccaaggattgccactgtatc

SEQ ID NO: 9 - protein, 130 aa, Artificial Sequence
  source: /note=“Description of Artificial Sequence Synthetic polypeptide”
HHHHHHHHGGSENLYFQGARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 10 - DNA, 591 bp, Artificial Sequence
  source: /note=“Description of Artificial Sequence Synthetic polynucleotide”
atgagattcccttccatctttacagcagtgttatttgctgctagttccgccctagcagctccagctaacacgactactgaagatgaaacagcccaaatcccagcagaagctgttattgactacagcgacttggagggtgacttcgacgcagctgctctccccctttctaattctactaataatggactgagttccacaaatactaccattgcctcaattgccgccaaggaggaaggtgtccaactggacaaaagagctagaaatggtgaccactgccctttaggtcccggcagatgttgtcgtttgcatactgtgagagcatcactggaggatctaggatgggctgattgggtgttgtctccaagggaggttcaggtaactatgtgtataggagcatgcccatcccagttcagggctgcaaacatgcacgctcaaatcaaaacaagccttcatcgtttgaaacctgatacagtaccggcaccatgttgtgttccagcttcatataaccctatggtcctgatccaaaagaccgacactggtgtttcgttgcaaacgtacgatgatttgttggctaaggattgccattgtatt

SEQ ID NO: 11 - protein, 112 aa, Artificial Sequence
  source: /note=“Description of Artificial Sequence Synthetic polypeptide”
ARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 12 - DNA, 819 bp, Artificial Sequence
  source: /note=“Description of Artificial Sequence Synthetic polynucleotide”
atgcatcaccatcatcatcaccaaaaacctgttggcgttgaagagccggtctacgatactgcaggtcgtcctctttttgggaatccgtccgaagtgcacccccagtcaaccctcaagcttccccatgaccgcggagaagatgacattgaaacaacgctgcgcgatctgcctcgtaaaggcgattgtcgctctggaaaccacctaggtccggtgtcgggcatttacattaaaccaggtcccgtctattaccaagactacactggtccggtttaccatcgtgcacctctggaattctttgatgaaacccaatttgaggaaaccactaaacgtattggccgtgtaaccggttcggacgggaaactgtaccacatctacgtggaggttgatggcgagatcctgctgaaacaggcgaagcgcggaacccctcgcaccctgaaatggacccgtaacaccactaactgtccactgtgggtcactagttgcgcacgcaacggtgatcattgtccgctgggtcctggtcgctgctgccgtctgcatacggtgcgtgcgagcctggaagatctgggctgggcagattgggtcctgtccccacgcgaggttcaagtgacgatgtgcattggtgcgtgcccgagccagttccgtgcggccaacatgcacgcacagattaagacctctctgcaccgtctgaaaccggacaccgtgccggctccgtgttgtgtcccggccagctataatccgatggttctgatccaaaagaccgacaccggcgttagcttgcagacttacgacgatctgttggcgaaagactgtcactgcatc

SEQ ID NO: 13 - protein, 273 aa, Artificial Sequence
  source: /note=“Description of Artificial Sequence Synthetic polypeptide”
MHHHHHHQKPVGVEEPVYDTAGRPLFGNPSEVHPQSTLKLPHDRGEDDIETTLRDLPRKGDCRSGNHLGPVSGIYIKPGPVYYQDYTGPVYHRAPLEFFDETQFEETTKRIGRVTGSDGKLYHIYVEVDGEILLKQAKRGTPRTLKWTRNTTNCPLWVTSCARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 14 - protein, 112 aa, Artificial Sequence; identical to SEQ ID NO: 11
  source: /note=“Description of Artificial Sequence Synthetic polypeptide”

SEQ ID NO: 15 - DNA, 2193 bp, Artificial Sequence
  source: /note=“Description of Artificial Sequence Synthetic polynucleotide”
atggagactgataccctgctcctctgggtgctgcttctctgggtccctggctcaaccggcgaagcccacaagtccgagatcgcccatcgctataatgctcttggagaacagcatttcaagggactggtgctgattgccttctcccagtacctccaaaaggccagctatgatgagcacgccaagctcgtccaagaagtcaccgactttgctaagacttgtgtggccgacgaaagcgctgccaattgcgataagtcactccatactctcttcggggacaagctgtgcgctattcccaacctccgcgagaattacggtgagctggccgactgttgcaccaaacaggagccagagcggaacgagtgcttccttcaacacaaagatgacaatccttcactgcctcctttcgaacggcccgaggcagaggcaatgtgcactagcttcaaggagaacccaaccaccttcatgggacactacctccatgaggtcgctagacggcatccctacttctatgccccagagcttctgtattatgcagaacagtacaatgagatcctgacccagtgctgtgctgaggctgataaggagagctgcctgaccccaaagctcgacggagtgaaggaaaaggctcttgtgtccagcgtgcggcagcgcatgaagtgctcttcaatgcagaagtttggggagcgcgccttcaaagcctgggccgtggccagactgtcccagacctttcctaatgctgactttgccgagatcaccaagctcgctactgacctgaccaaggtcaacaaagagtgttgccacggagatctgctcgaatgcgccgacgaccgcgctgagcttgctaagtacatgtgcgaaaaccaggcaaccatttctagcaagctgcagacctgttgtgataagcctctgctgaagaaagcccattgcctcagcgaggtcgaacatgacactatgccggcagacctccccgctatcgccgctgacttcgtggaggaccaagaagtgtgcaagaattacgccgaggctaaggacgtgttccttggtactttcctctacgagtatagccggaggcaccctgactacagcgtgtctcttctgcttcggctcgccaagaagtacgaagccaccctcgaaaaatgctgcgccgaagcaaatccgccagcttgttacgggactgtgctggctgagtttcagcccctggtggaagagcccaagaacctcgtcaagaccaactgcgacctttacgagaaactgggtgaatacgggtttcagaatgccattctggtgcggtacacccagaaggcaccacaagtgtccaccccaacccttgtcgaggcagcccgcaaccttggacgcgtcgggaccaagtgttgtaccctgcccgaggaccaacgcctgccctgcgtcgaggactaccttagcgccattctgaacagagtctgtctgctccatgaaaagacccctgtgtctgagcacgtgaccaagtgctgttcaggctcactggtggagaggaggccttgcttttctgccctgaccgtggacgaaacctacgtgcccaaggagttcaaagctgaaaccttcactttccattcagacatctgtaccctccccgaaaaggaaaagcaaatcaagaagcagaccgcccttgctgaactggtgaagcacaagccaaaggccaccgccgaacaactcaagactgtgatggacgacttcgctcagttcctcgacacttgctgcaaagccgccgacaaagatacctgtttctcaaccgaggggccgaacctggtgactagagccaaggacgccctggccggaggaggtggttctggcggtggtggttccggcggaggaggatctgccaggaatggagatcactgcccactcggaccgggacggtgttgtcgcctgcacactgtgcgcgcatctcttgaggatctgggatgggctgattgggtgctctctcccagagaggtgcaagtcaccatgtgcattggcgcctgcccctcccaattcagggcagctaacatgcatgctcagatcaagactagcctgcacaggctgaagcccgacactgtccctgccccatgttgtgtgccggcctcctataacccaatggtcctgatccaaaagaccgataccggagtgtcacttcagacttacgacgatctgcttgcaaaagactgccattgcatc

SEQ ID NO: 16 - protein, 711 aa, Artificial Sequence
  source: /note=“Description of Artificial Sequence Synthetic polypeptide”
EAHKSEIAHRYNALGEQHFKGLVLIAFSQYLQKASYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRAKDALAGGGGSGGGGSGGGGSARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 17 - DNA, 2151 bp, Artificial Sequence
  source: /note=“Description of Artificial Sequence Synthetic polynucleotide”
atggagactgatacccttctgctctgggtgcttctgctgtgggtgccaggatccaccggcgaagcccataagtcggaaatcgcacatcggtacaacgcgctcggggaacagcacttcaaaggccttgtcctgatcgcgttctcccaataccttcaaaaggcctcgtacgatgaacatgctaagctcgtccaagaggtgaccgacttcgcaaagacttgtgtggccgatgagtcggcagccaactgcgacaagagcctccacactctcttcggagacaagctgtgcgcaattcctaatctgcgcgagaattacggggaactggcggactgctgtactaagcaagagccggaacgcaatgagtgcttcctccagcataaggacgacaacccttccctccctcccttcgaacgcccagaggccgaagcgatgtgtacctccttcaaggaaaacccgaccacgtttatgggacattacctccacgaagtcgccagacggcatccctacttctacgcgcctgagctgctctattacgccgaacagtacaacgagatcctgacgcagtgttgcgctgaggcagacaaggagagctgcttgaccccgaaactcgatggagtgaaggagaaggccctggtgagcagcgtgcgccagcggatgaagtgctcatcgatgcagaagttcggcgagagagctttcaaggcgtgggccgtggccaggctgtcacagacctttccaaacgcggatttcgcagagatcaccaagctggccactgacctcactaaagtcaacaaggaatgctgccacggagatctcttggaatgtgccgatgacagggccgaattggctaagtacatgtgcgaaaatcaagctaccattagctcgaagctgcagacgtgctgcgataagccgctgctgaagaaggctcattgcctgtccgaggtggagcacgacaccatgccagccgacctcccggccatcgcagcagattttgtggaggatcaggaagtgtgcaagaattacgcagaagctaaggatgtgtttcttgggacttttctctacgagtacagccggagacacccggactatagcgtgtccctgctgctgcgcttggctaagaaatacgaagctacccttgaaaaatgctgcgcagaggccaaccctccggcttgctacggaactgtgctggctgagttccagccgctcgtcgaagaaccgaagaatctcgtgaaaacgaactgcgatctgtacgagaaattgggagagtatggatttcaaaatgccattctggtccgctacactcagaaagctccacaagtctccacgccgaccctggtcgaagcggcgaggaaccttggacgcgtgggaaccaagtgctgtaccctgccggaggaccagcgccttccgtgcgtcgaggattacttgtcagcgatcctcaaccgcgtgtgcttgcttcatgaaaagactcccgtgtcggaacacgtgacgaagtgctgctccggttcgctggtggaaagacgcccgtgcttctcggccctgactgtggacgaaacctacgtcccaaaagagttcaaggctgaaaccttcactttccactcggacatctgcactctccccgaaaaggaaaaacagatcaagaagcagactgccctggcagagctggtgaaacacaagcccaaggcgacggccgaacagctgaaaaccgtgatggacgactttgcccaattcctcgacacttgttgtaaagcagccgataaggacacttgcttctccactgagggccctaacctggtcacccgggctaaggacgcgctcgcgggaggaggtggcagcggaggaggcggtagcggaggcggagggtcatgtcggctgcacaccgtgcgggcatcgcttgaagatttgggatgggccgactgggtgctgtcaccgcgggaagtgcaagtgaccatgtgcatcggcgcctgcccgtcgcagtttagagcagcgaatatgcacgcgcaaatcaagacttcgctgcacagactgaagccggatactgtccctgcaccatgctgcgtccctgcctcatacaacccaatggtgctgatccagaaaaccgacaccggagtgtcgctccagacttacgacgaccttctggccaaggactgtcattgtatc

SEQ ID NO: 18 - protein, 697 aa, Artificial Sequence
  source: /note=“Description of Artificial Sequence Synthetic polypeptide”
EAHKSEIAHRYNALGEQHFKGLVLIAFSQYLQKASYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRAKDALAGGGGSGGGGSGGGGSCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 19 - DNA, 2154 bp, Artificial Sequence
  source: /note=“Description of Artificial Sequence Synthetic polynucleotide”
atggaaactgacactttgctgctttgggttctgctcctttgggtccctggatcaactggtgatgctcacaagtccgaagtggcccaccgtttcaaggatctgggtgaggaaaacttcaaggctctcgtcctgatcgcatttgcgcagtacctccagcagtcgccattcgaggaccatgtgaaactcgtcaacgaagtgaccgagtttgctaagacttgcgtcgctgacgagtcagcagagaattgtgacaaatccctgcacaccctgttcggcgataagctctgcactgtggccaccctccgggaaacttacggcgagatggcggattgttgcgcgaaacaggaacccgagcgcaatgagtgtttcctgcagcacaaggacgacaacccgaacctcccacggctggtgaggccggaagtggacgtcatgtgcaccgcatttcatgacaacgaagagactttcctgaagaagtacctgtacgaaatcgctcggagacatccgtacttctacgcgccggaactcctcttctttgctaagcggtacaaggcagcctttactgaatgctgccaggccgccgacaaagcggcgtgtctgctgccgaaactggacgagctgagagatgaaggaaaggctagctcggccaagcagcggttgaaatgcgcatcgctccaaaagttcggagaaagagctttcaaggcctgggcagtggcgcggctctcgcagcgcttccctaaggcagagttcgccgaggtcagcaagttggtgacggacctgactaaagtccataccgaatgttgccacggagatctgctcgaatgcgccgatgaccgggccgacctggcgaagtacatttgtgagaaccaagattcaatttcgagcaagttgaaggagtgctgcgaaaagccgttgcttgagaagtcgcactgcatcgcagaagtcgaaaacgatgagatgcctgccgacttgccgagcctggccgccgatttcgtggagagcaaagacgtgtgcaaaaattacgccgaggccaaggacgtgttcctgggaatgttcctgtacgaatatgcgcgacgccacccagactacagcgtggtcctgctgctccgccttgctaaaacttacgaaaccacgctggagaaatgctgtgccgcagccgacccacatgagtgctacgcaaaggtgttcgacgagtttaaaccccttgtggaagaaccgcagaatctgatcaagcagaactgcgagctgttcgaacaactcggagaatacaagttccagaacgctctgcttgtcagatacaccaagaaagtgccgcaagtgtccacgccaaccctggtggaagtctcacgcaacctgggaaaggtcggaagcaagtgctgtaagcatcctgaagcaaagagaatgccatgcgcggaggactacctgtccgttgtcctgaatcaactctgcgtgctgcacgagaaaactccagtgtcggaccgcgtcaccaagtgttgcacggaatcgctcgtgaatcgcaggccgtgcttctccgccctggaagttgatgagacttacgtcccgaaagagtttcaggccgaaaccttcacctttcacgcggacatctgcactctctctgaaaaggaaagacaaatcaagaagcagactgccctggtggagctggtcaagcataaaccaaaggcgaccaaggaacagttgaaagccgtgatggacgatttcgctgccttcgtggagaagtgctgcaaggccgacgacaaggaaacttgctttgccgaggaaggaaagaaactggtggccgcatcccaagccgcgctgggactcggaggtggtgggtcggggggagggggctccggcggcggagggtcatgtcgcctccacaccgtgcgggcgtccctggaagatctgggatgggccgattgggtgctgtccccgcgcgaggtgcaagtgactatgtgtatcggcgcgtgcccatcacaattcagggcagccaatatgcatgcacagatcaaaacctcgctccaccgccttaagccggacaccgtgcccgcgccctgctgcgtgcctgcttcctataaccctatggttctgatccaaaagaccgataccggcgtgagcctgcagacctacgatgatctcctggccaaggactgccactgtatc

SEQ ID NO: 20 - protein, 698 aa, Artificial Sequence
  source: /note=“Description of Artificial Sequence Synthetic polypeptide”
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLGGGGSGGGGSGGGGSCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 21 - DNA, 2193 bp, Artificial Sequence
  source: /note=“Description of Artificial Sequence Synthetic polynucleotide”
atggagactgatacccttctgctctgggtgcttctgctgtgggtgccaggatccaccggcgaagcccataagtcggaaatcgcacatcggtacaacgcgctcggggaacagcacttcaaaggccttgtcctgatcgcgttctcccaataccttcaaaaggcctcgtacgatgaacatgctaagctcgtccaagaggtgaccgacttcgcaaagacttgtgtggccgatgagtcggcagccaactgcgacaagagcctccacactctcttcggagacaagctgtgcgcaattcctaatctgcgcgagaattacggggaactggcggactgctgtactaagcaagagccggaacgcaatgagtgcttcctccagcataaggacgacaacccttccctccctcccttcgaacgcccagaggccgaagcgatgtgtacctccttcaaggaaaacccgaccacgtttatgggacattacctccacgaagtcgccagacggcatccctacttctacgcgcctgagctgctctattacgccgaacagtacaacgagatcctgacgcagtgttgcgctgaggcagacaaggagagctgcttgaccccgaaactcgatggagtgaaggagaaggccctggtgagcagcgtgcgccagcggatgaagtgctcatcgatgcagaagttcggcgagagagctttcaaggcgtgggccgtggccaggctgtcacagacctttccaaacgcggatttcgcagagatcaccaagctggccactgacctcactaaagtcaacaaggaatgctgccacggagatctcttggaatgtgccgatgacagggccgaattggctaagtacatgtgcgaaaatcaagctaccattagctcgaagctgcagacgtgctgcgataagccgctgctgaagaaggctcattgcctgtccgaggtggagcacgacaccatgccagccgacctcccggccatcgcagcagattttgtggaggatcaggaagtgtgcaagaattacgcagaagctaaggatgtgtttcttgggacttttctctacgagtacagccggagacacccggactatagcgtgtccctgctgctgcgcttggctaagaaatacgaagctacccttgaaaaatgctgcgcagaggccaaccctccggcttgctacggaactgtgctggctgagttccagccgctcgtcgaagaaccgaagaatctcgtgaaaacgaactgcgatctgtacgagaaattgggagagtatggatttcaaaatgccattctggtccgctacactcagaaagctccacaagtctccacgccgaccctggtcgaagcggcgaggaaccttggacgcgtgggaaccaagtgctgtaccctgccggaggaccagcgccttccgtgcgtcgaggattacttgtcagcgatcctcaaccgcgtgtgcttgcttcatgaaaagactcccgtgtcggaacacgtgacgaagtgctgctccggttcgctggtggaaagacgcccgtgcttctcggccctgactgtggacgaaacctacgtcccaaaagagttcaaggctgaaaccttcactttccactcggacatctgcactctccccgaaaaggaaaaacagatcaagaagcagactgccctggcagagctggtgaaacacaagcccaaggcgacggccgaacagctgaaaaccgtgatggacgactttgcccaattcctcgacacttgttgtaaagcagccgataaggacacttgcttctccactgagggccctaacctggtcacccgggctaaggacgcgctcgcgggaggaggtggcagcggaggaggcggtagcggaggcggagggagcgctagaaacggcgaccacagcccgttggggccaggtagatcatgtcggctgcacaccgtgcgggcatcgcttgaagatttgggatgggccgactgggtgctgtcaccgcgggaagtgcaagtgaccatgtgcatcggcgcctgcccgtcgcagtttagagcagcgaatatgcacgcgcaaatcaagacttcgctgcacagactgaagccggatactgtccctgcaccatgctgcgtccctgcctcatacaacccaatggtgctgatccagaaaaccgacaccggagtgtcgctccagacttacgacgaccttctggccaaggactgtcattgtatc

SEQ ID NO: 22 - protein, 711 aa, Artificial Sequence
  source: /note=“Description of Artificial Sequence Synthetic polypeptide”
EAHKSEIAHRYNALGEQHFKGLVLIAFSQYLQKASYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRAKDALAGGGGSGGGGSGGGGSARNGDHSPLGPGRSCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 23 - DNA, 2193 bp, Artificial Sequence
  source: /note=“Description of Artificial Sequence Synthetic polynucleotide”
atggagactgatacccttctgctctgggtgcttctgctgtgggtgccaggatccaccggcgaagcccataagtcggaaatcgcacatcggtacaacgcgctcggggaacagcacttcaaaggccttgtcctgatcgcgttctcccaataccttcaaaaggcctcgtacgatgaacatgctaagctcgtccaagaggtgaccgacttcgcaaagacttgtgtggccgatgagtcggcagccaactgcgacaagagcctccacactctcttcggagacaagctgtgcgcaattcctaatctgcgcgagaattacggggaactggcggactgctgtactaagcaagagccggaacgcaatgagtgcttcctccagcataaggacgacaacccttccctccctcccttcgaacgcccagaggccgaagcgatgtgtacctccttcaaggaaaacccgaccacgtttatgggacattacctccacgaagtcgccagacggcatccctacttctacgcgcctgagctgctctattacgccgaacagtacaacgagatcctgacgcagtgttgcgctgaggcagacaaggagagctgcttgaccccgaaactcgatggagtgaaggagaaggccctggtgagcagcgtgcgccagcggatgaagtgctcatcgatgcagaagttcggcgagagagctttcaaggcgtgggccgtggccaggctgtcacagacctttccaaacgcggatttcgcagagatcaccaagctggccactgacctcactaaagtcaacaaggaatgctgccacggagatctcttggaatgtgccgatgacagggccgaattggctaagtacatgtgcgaaaatcaagctaccattagctcgaagctgcagacgtgctgcgataagccgctgctgaagaaggctcattgcctgtccgaggtggagcacgacaccatgccagccgacctcccggccatcgcagcagattttgtggaggatcaggaagtgtgcaagaattacgcagaagctaaggatgtgtttcttgggacttttctctacgagtacagccggagacacccggactatagcgtgtccctgctgctgcgcttggctaagaaatacgaagctacccttgaaaaatgctgcgcagaggccaaccctccggcttgctacggaactgtgctggctgagttccagccgctcgtcgaagaaccgaagaatctcgtgaaaacgaactgcgatctgtacgagaaattgggagagtatggatttcaaaatgccattctggtccgctacactcagaaagctccacaagtctccacgccgaccctggtcgaagcggcgaggaaccttggacgcgtgggaaccaagtgctgtaccctgccggaggaccagcgccttccgtgcgtcgaggattacttgtcagcgatcctcaaccgcgtgtgcttgcttcatgaaaagactcccgtgtcggaacacgtgacgaagtgctgctccggttcgctggtggaaagacgcccgtgcttctcggccctgactgtggacgaaacctacgtcccaaaagagttcaaggctgaaaccttcactttccactcggacatctgcactctccccgaaaaggaaaaacagatcaagaagcagactgccctggcagagctggtgaaacacaagcccaaggcgacggccgaacagctgaaaaccgtgatggacgactttgcccaattcctcgacacttgttgtaaagcagccgataaggacacttgcttctccactgagggccctaacctggtcacccgggctaaggacgcgctcgcgggaggaggtggcagcggaggaggcggtagcggaggcggagggagcgctagaaacggcgaccactgtccactggggccaggtcggtgctgtcggctgcacaccgtgcgggcatcgcttgaagatttgggatgggccgactgggtgctgtcaccgcgggaagtgcaagtgaccatgtgcatcggcgcctgcccgtcgcagtttagagcagcgaatatgcacgcgcaaatcaagacttcgctgcacagactgaagccggatactgtccctgcaccatcatgcgtccctgcctcatacaacccaatggtgctgatccagaaaaccgacaccggagtgtcgctccagacttacgacgaccttctggccaaggactgtcattgtatc

SEQ ID NO: 24 - protein, 711 aa, Artificial Sequence
  source: /note=“Description of Artificial Sequence Synthetic polypeptide”
EAHKSEIAHRYNALGEQHFKGLVLIAFSQYLQKASYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRAKDALAGGGGSGGGGSGGGGSARNGDHCPLGPGRCCRLHTVRASLEDLGWADWVLSPREVQVTMCIGACPSQFRAANMHAQIKTSLHRLKPDTVPAPSCVPASYNPMVLIQKTDTGVSLQTYDDLLAKDCHCI

SEQ ID NO: 25 - DNA, 2196 bp, Artificial Sequence
  source: /note=“Description of Artificial Sequence Synthetic polynucleotide”
atggaaaccgatactctgctgctgtgggtgcttcttctttgggtgccgggatcaaccggcgatgcccacaagtcggaggtggcccatcggtttaaggacctcggggaggagaacttcaaagccctggtcctcatcgccttcgcccaatacctccagcagtgtccattcgaagatcacgtgaagctcgtgaacgaagtgactgaatttgccaagacttgtgtcgcagacgaaagcgccgaaaactgcgacaagtcgttgcatactctcttcggggataagctgtgcactgtcgcaacccttagagagacttacggtgaaatggctgattgctgcgccaaacaagagccggagcgcaacgagtgcttcctccaacataaggacgacaaccccaacctcccacgcctggtgcggcctgaggtcgacgtcatgtgcaccgctttccatgacaatgaggagacttttctcaagaagtatctgtacgagatcgcccggaggcacccatacttttatgcaccggagctccttttcttcgctaagcggtacaaggcggcgttcactgaatgctgtcaggcagcagacaaggcagcatgcctcctgccgaaactggacgaacttcgcgacgagggtaaagcgtcgtccgccaagcagcgccttaagtgcgcctcgttgcagaagtttggtgaacgcgcattcaaagcgtgggccgtcgcaagactttcgcagcggttcccaaaagcggagtttgccgaggtgtccaaactggtcaccgacctgaccaaggtccacaccgagtgctgccacggcgatctgctcgaatgcgccgacgaccgggctgatctcgcaaagtacatttgcgagaaccaagactcgatctcgtcaaaactgaaggaatgctgcgagaagccgctgttggaaaagagccattgtatcgccgaagtggagaacgatgaaatgcctgctgatctgccaagcctcgccgcagactttgtggagagcaaagacgtgtgcaagaactacgccgaagcgaaggacgtgtttctcgggatgttcctctacgagtacgcgcgcaggcaccctgactactcagtggtcctgctgttgcggctggccaaaacttacgaaaccaccctcgaaaagtgctgcgcggctgccgatccacatgaatgctacgcaaaggtgttcgatgaatttaagcctctggtggaggaaccacagaacctgatcaagcaaaattgtgaactgtttgaacagctgggagagtacaaatttcagaatgccctgctggtcagatacactaagaaggtgccccaagtctccactccaaccctcgtggaggtgtcacggaatctcggcaaagtgggcagcaaatgctgtaagcacccggaagcaaagaggatgccctgcgctgaagattacctgtccgtggtgctgaatcagctttgtgtgctgcacgaaaagacgcctgtctccgaccgggtgaccaagtgctgtaccgaatcgctcgtgaatcgcagaccctgcttctccgctctcgaagtggacgaaacttacgtcccgaaggagttcaatgcggaaaccttcaccttccacgcggacatctgtaccctgagcgaaaaagagcggcagatcaagaaacagactgccctggtggaactggtgaagcacaagccgaaggcaacgaaggagcagctgaaggcggtgatggatgactttgcagccttcgtggaaaagtgttgcaaggcagatgataaagaaacctgtttcgcggaagaggggaagaagttggtggctgccagccaggccgctctcggactgggaggtggaggatcaggaggcggaggctccggaggaggaggctcggctcgcaatggcgatcattgcccgctcggaccgggacgctgctgcagactgcataccgtccgcgcttccttggaagatctgggatgggcggattgggtgttgtcaccaagagaggtgcaagtgacgatgtgtatcggtgcgtgcccttcacagttccgcgctgcgaacatgcatgcccaaatcaagaccagcctgcaccggctgaagccggacactgtcccagctccatgttgcgtgcccgcatcgtacaacccgatggtgctcatccagaaaactgacactggagtctcactgcaaacgtacgacgatttgctcgccaaagattgccactgcatt

SEQ ID NO: 26 - protein, 712 aa, Artificial Sequence
  source: /note=“Description of Artificial Sequence Synthetic polypeptide”
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 27 - DNA, 2169 bp, Artificial Sequence
  source: /note=“Description of Artificial Sequence Synthetic polynucleotide”
atggccctccctgtcaccgccctgctgcttccgctggctcttctgctccacgccgctcggcccgatgctcataaatcagaagtggcgcacagattcaaggacctcggagaagaaaactttaaagcactggtgctgatcgccttcgcacaatacttgcagcagtgcccgttcgaagatcacgtgaaactggtcaacgaagtgaccgagttcgctaagacctgtgtcgctgacgagagcgcggaaaactgcgacaagtcccttcacacgctgttcggcgataagctctgcacggtcgcgactctgagggaaacctacggagagatggcagattgctgtgcaaagcaggaacctgagaggaacgaatgtttcctgcaacataaggacgacaacccaaatcttccgcgcctcgtgcgtccggaggtggacgtgatgtgcacggccttccatgataatgaggaaactttcctgaaaaagtacctctacgaaatcgcccggagacacccgtatttctacgccccggagcttctgttcttcgcaaagcgctacaaggcggcttttactgagtgctgccaagctgccgacaaagccgcatgcctgctgccaaagctcgatgaactcagggacgagggaaaggcatcctccgcaaagcagcgcctgaaatgcgcctcactgcaaaagtttggagaacgcgcattcaaggcctgggcggtggcccggctcagccagagattccccaaggccgagtttgccgaggtgtccaagctcgttactgatctgaccaaagtccacaccgaatgctgtcatggagatcttttggagtgcgccgacgacagagcggacctggccaagtacatctgcgaaaaccaggattcgatctcatctaagctcaaggagtgctgcgaaaaacccctgttggaaaagtcgcactgtattgcggaagtggagaacgacgagatgcctgcagacttgccgtcactggcggctgacttcgtggagtcgaaggacgtgtgcaaaaactacgcggaagcgaaggatgtctttctgggaatgttcctgtacgaatacgcacggcgccatccggactactcagttgtgctgttgctccgccttgctaagacttacgaaactaccttggagaaatgctgcgccgccgccgatcctcacgaatgttacgcaaaagtgttcgacgagtttaagcctctcgtggaagaacctcagaatctgatcaagcagaactgtgaactgttcgagcagctcggggaatacaagttccagaatgcgctgctcgtccggtatactaagaaagtgccacaagtgtccaccccgactctggtcgaagtgtcgcgcaatctggggaaagtcggatcgaagtgctgcaagcatccggaggcgaaacgaatgccgtgcgcggaggattacctgtcggtggtgctgaaccagctctgcgtgctgcatgaaaagaccccggtgtccgaccgggtcaccaagtgttgcactgagtccctcgtgaaccggcgcccttgcttctcggccctcgaagtcgatgagacttacgtgccaaaagagtttaatgccgaaaccttcacctttcacgctgacatctgcactttgagcgaaaaggaaagacagattaagaagcagacggccctggtggaactcgtcaaacataaacccaaagctacgaaagagcagctgaaagcagttatggacgatttcgccgctttcgtggaaaaatgctgcaaggccgacgataaggaaacttgtttcgccgaggaggggaagaagctggtcgcagcaagccaagccgctctgggtcttggcggtggaggcagcgcgaggaatggcgaccactgcccattgggaccgggacggtgttgcagactccacactgtccgggcttcactcgaggacctgggttgggccgactgggtgctgtcgccccgggaagtccaggtcaccatgtgcatcggagcgtgcccgagccaatttcgcgccgcgaacatgcacgcccagatcaagacctcgctgcaccgcctgaagcctgacaccgtgccagccccctgctgtgtgccggcctcctacaacccaatggtgctcatccaaaagaccgataccggcgtgagcctgcaaacttacgatgatcttctggccaaggactgtcactgcatc

SEQ ID NO: 28 - protein, 702 aa, Artificial Sequence
  source: /note=“Description of Artificial Sequence Synthetic polypeptide”
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 29 - DNA, 2169 bp, Artificial Sequence
  source: /note=“Description of Artificial Sequence Synthetic polynucleotide”
atggccctccctgtcaccgccctgctgcttccgctggctcttctgctccacgccgctcggcccgatgctcataaatcagaagtggcgcacagattcaaggacctcggagaagaaaactttaaagcactggtgctgatcgccttcgcacaatacttgcagcagtgcccgttcgaagatcacgtgaaactggtcaacgaagtgaccgagttcgctaagacctgtgtcgctgacgagagcgcggaaaactgcgacaagtcccttcacacgctgttcggcgataagctctgcacggtcgcgactctgagggaaacctacggagagatggcagattgctgtgcaaagcaggaacctgagaggaacgaatgtttcctgcaacataaggacgacaacccaaatcttccgcgcctcgtgcgtccggaggtggacgtgatgtgcacggccttccatgataatgaggaaactttcctgaaaaagtacctctacgaaatcgcccggagacacccgtatttctacgccccggagcttctgttcttcgcaaagcgctacaaggcggcttttactgagtgctgccaagctgccgacaaagccgcatgcctgctgccaaagctcgatgaactcagggacgagggaaaggcatcctccgcaaagcagcgcctgaaatgcgcctcactgcaaaagtttggagaacgcgcattcaaggcctgggcggtggcccggctcagccagagattccccaaggccgagtttgccgaggtgtccaagctcgttactgatctgaccaaagtccacaccgaatgctgtcatggagatcttttggagtgcgccgacgacagagcggacctggccaagtacatctgcgaaaaccaggattcgatctcatctaagctcaaggagtgctgcgaaaaacccctgttggaaaagtcgcactgtattgcggaagtggagaacgacgagatgcctgcagacttgccgtcactggcggctgacttcgtggagtcgaaggacgtgtgcaaaaactacgcggaagcgaaggatgtctttctgggaatgttcctgtacgaatacgcacggcgccatccggactactcagttgtgctgttgctccgccttgctaagacttacgaaactaccttggagaaatgctgcgccgccgccgatcctcacgaatgttacgcaaaagtgttcgacgagtttaagcctctcgtggaagaacctcagaatctgatcaagcagaactgtgaactgttcgagcagctcggggaatacaagttccagaatgcgctgctcgtccggtatactaagaaagtgccacaagtgtccaccccgactctggtcgaagtgtcgcgcaatctggggaaagtcggatcgaagtgctgcaagcatccggaggcgaaacgaatgccgtgcgcggaggattacctgtcggtggtgctgaaccagctctgcgtgctgcatgaaaagaccccggtgtccgaccgggtcaccaagtgttgcactgagtccctcgtgaaccggcgcccttgcttctcggccctcgaagtcgatgagacttacgtgccaaaagagtttaatgccgaaaccttcacctttcacgctgacatctgcactttgagcgaaaaggaaagacagattaagaagcagacggccctggtggaactcgtcaaacataaacccaaagctacgaaagagcagctgaaagcagttatggacgatttcgccgctttcgtggaaaaatgctgcaaggccgacgataaggaaacttgtttcgccgaggaggggaagaagctggtcgcagcaagccaagccgctctgggtcttggcccaccgggcagcgcgaggaatggcgaccactgcccattgggaccgggacggtgttgcagactccacactgtccgggcttcactcgaggacctgggttgggccgactgggtgctgtcgccccgggaagtccaggtcaccatgtgcatcggagcgtgcccgagccaatttcgcgccgcgaacatgcacgcccagatcaagacctcgctgcaccgcctgaagcctgacaccgtgccagccccctgctgtgtgccggcctcctacaacccaatggtgctcatccaaaagaccgataccggcgtgagcctgcaaacttacgatgatcttctggccaaggactgtcactgcatc

SEQ ID NO: 30 - protein, 702 aa, Artificial Sequence
  source: /note=“Description of Artificial Sequence Synthetic polypeptide”
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGPPGSARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 31 - DNA, 2154 bp, Artificial Sequence
  source: /note=“Description of Artificial Sequence Synthetic polynucleotide”
atggccctccctgtcaccgccctgctgcttccgctggctcttctgctccacgccgctcggcccgatgctcataaatcagaagtggcgcacagattcaaggacctcggagaagaaaactttaaagcactggtgctgatcgccttcgcacaatacttgcagcagtgcccgttcgaagatcacgtgaaactggtcaacgaagtgaccgagttcgctaagacctgtgtcgctgacgagagcgcggaaaactgcgacaagtcccttcacacgctgttcggcgataagctctgcacggtcgcgactctgagggaaacctacggagagatggcagattgctgtgcaaagcaggaacctgagaggaacgaatgtttcctgcaacataaggacgacaacccaaatcttccgcgcctcgtgcgtccggaggtggacgtgatgtgcacggccttccatgataatgaggaaactttcctgaaaaagtacctctacgaaatcgcccggagacacccgtatttctacgccccggagcttctgttcttcgcaaagcgctacaaggcggcttttactgagtgctgccaagctgccgacaaagccgcatgcctgctgccaaagctcgatgaactcagggacgagggaaaggcatcctccgcaaagcagcgcctgaaatgcgcctcactgcaaaagtttggagaacgcgcattcaaggcctgggcggtggcccggctcagccagagattccccaaggccgagtttgccgaggtgtccaagctcgttactgatctgaccaaagtccacaccgaatgctgtcatggagatcttttggagtgcgccgacgacagagcggacctggccaagtacatctgcgaaaaccaggattcgatctcatctaagctcaaggagtgctgcgaaaaacccctgttggaaaagtcgcactgtattgcggaagtggagaacgacgagatgcctgcagacttgccgtcactggcggctgacttcgtggagtcgaaggacgtgtgcaaaaactacgcggaagcgaaggatgtctttctgggaatgttcctgtacgaatacgcacggcgccatccggactactcagttgtgctgttgctccgccttgctaagacttacgaaactaccttggagaaatgctgcgccgccgccgatcctcacgaatgttacgcaaaagtgttcgacgagtttaagcctctcgtggaagaacctcagaatctgatcaagcagaactgtgaactgttcgagcagctcggggaatacaagttccagaatgcgctgctcgtccggtatactaagaaagtgccacaagtgtccaccccgactctggtcgaagtgtcgcgcaatctggggaaagtcggatcgaagtgctgcaagcatccggaggcgaaacgaatgccgtgcgcggaggattacctgtcggtggtgctgaaccagctctgcgtgctgcatgaaaagaccccggtgtccgaccgggtcaccaagtgttgcactgagtccctcgtgaaccggcgcccttgcttctcggccctcgaagtcgatgagacttacgtgccaaaagagtttaatgccgaaaccttcacctttcacgctgacatctgcactttgagcgaaaaggaaagacagattaagaagcagacggccctggtggaactcgtcaaacataaacccaaagctacgaaagagcagctgaaagcagttatggacgatttcgccgctttcgtggaaaaatgctgcaaggccgacgataaggaaacttgtttcgccgaggaggggaagaagctggtcgcagcaagccaagccgctctgggtcttgcgaggaatggcgaccactgcccattgggaccgggacggtgttgcagactccacactgtccgggcttcactcgaggacctgggttgggccgactgggtgctgtcgccccgggaagtccaggtcaccatgtgcatcggagcgtgcccgagccaatttcgcgccgcgaacatgcacgcccagatcaagacctcgctgcaccgcctgaagcctgacaccgtgccagccccctgctgtgtgccggcctcctacaacccaatggtgctcatccaaaagaccgataccggcgtgagcctgcaaacttacgatgatcttctggccaaggactgtcactgcatc

SEQ ID NO: 32 - protein, 697 aa, Artificial Sequence
  source: /note=“Description of Artificial Sequence Synthetic polypeptide”
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 33 - DNA, 1035 bp, Artificial Sequence
  source: /note=“Description of Artificial Sequence Synthetic polynucleotide”
atggccctccctgtcaccgccctgctgcttccgctggctcttctgctccacgccgctcggcccgaagctcataagtcagaaatcgcccatagatacaacgacctcggggaacagcactttaaaggactcgtgttgattgcattcagccagtacctccaaaagtgcagctacgacgagcatgcgaagctggtgcaggaagtcaccgacttcgccaaaacttgcgtcgctgatgagtcggcggcaaactgcgacaaatcgctccacaccctgtttggcgataagctgtgtgcgatcccgaatcttcgagagaattacggagaacttgcagactgctgcaccaagcaggaaccggaacgcaacgagtgcttcctccaacacaaggatgacaacccatctctgccccctttcgaacggccggaggcggaagccatgtgcactagctttaaggagaatccaactacgttcatggggcattacctccacgaggtcgccaggcggcatccatacttctacgccccggaactgctgtactatgccgagcagtacaacgaaatcctgacgcagtgctgtgccgaggctgataaggaatcatgcctgaccccaaagctggacggagtgaaagaaaaggcgctcgtgtcgtccgtgagacaacgcggtggaggaggctccggcggcggaggctcgggagggggaggttcagcacggaacggcgaccactgccctttggggccgggacgctgttgccggcttcacactgtgcgcgcgtccctcgaggatttgggatgggcagattgggtgctgagcccgagagaggtccaggtcaccatgtgtatcggtgcctgcccgagccagttcagggctgccaacatgcacgcgcagatcaaaacttcgctgcatcgcctgaaaccagacaccgttccggcaccctgttgcgtgcctgcctcctacaatcctatggtgctgattcaaaagaccgacaccggagtgtccctgcaaacttacgacgatctgctcgccaaggactgccactgtatc

SEQ ID NO: 34 - protein, 324 aa, Artificial Sequence
  source: /note=“Description of Artificial Sequence Synthetic polypeptide”
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRGGGGSGGGGSGGGGSARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 35 - DNA, 1122 bp, Artificial Sequence
  source: /note=“Description of Artificial Sequence Synthetic polynucleotide”
atggagacagacacgctccttttgtgggtactgctgctttgggtccctgggtcgacaggggataagacccacacgtgccctccctgtccagcacccgagttgctcggtgggccatccgtgtttttgtttcctcccaagcccaaagacacgttgatgattagccgcactcccgaggtaacgtgcgtagtggtggatgtgtcacatgaggacccggaggtgaagttcaattggtacgtggacggagtcgaagtgcacaacgcaaagacgaaaccccgagaggaacagtacaactcgacctatcgcgtagtgagcgtactgactgtgttgcatcaggattggcttaacggaaaagagtacaagtgtaaagtatccaataaggccctcccagcgcctattgaaaagacaatcagcaaagcgaaggggcagcctcgcgaaccgcaagtatataccctcccgcctagccgggacgaattgactaagaatcaggtcagcctcacatgtctggtcaaaggcttttacccgtcagatatcgcggtcgagtgggagtccaatgggcagccggaaaacaattacaagacaacgccgccagtcttggactcagacgggtcgtttttcctctactcgaaactgacggtggacaagtcccgatggcagcagggaaatgtattcagctgttcggtcatgcacgaggcgctccacaatcattatacacaaaagtcgctgtccctgtcgccgggaaagggaggtggcgggtccggcggaggaggatcaggtggtggaggttcagccagaaacggtgatcattgcccacttggacccgggaggtgctgtcggcttcacactgtcagggcatcactcgaagatctcgggtgggcggactgggtgctttcgcccagagaagtgcaagtcactatgtgcattggtgcgtgcccgtcgcaattcagagctgccaacatgcatgcccagatcaaaacgagcttgcaccggctgaaacccgacacagtccccgctccgtgctgcgtgccggcgtcgtataaccccatggtcctcatccagaaaaccgatacgggagtgtcattgcagacatatgatgaccttttggccaaggattgccactgtatc

SEQ ID NO: 36 - protein, 354 aa, Artificial Sequence
  source: /note=“Description of Artificial Sequence Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 37 - DNA, 2196 bp, Artificial Sequence
  source: /note=“Description of Artificial Sequence Synthetic polynucleotide”
atggaaaccgatactctgctgctgtgggtgcttcttctttgggtgccgggatcaaccggcgatgcccacaagtcggaggtggcccatcggtttaaggacctcggggaggagaacttcaaagccctggtcctcatcgccttcgcccaatacctccagcagtgtccattcgaagatcacgtgaagctcgtgaacgaagtgactgaatttgccaagacttgtgtcgcagacgaaagcgccgaaaactgcgacaagtcgttgcatactctcttcggggataagctgtgcactgtcgcaacccttagagagacttacggtgaaatggctgattgctgcgccaaacaagagccggagcgcaacgagtgcttcctccaacataaggacgacaaccccaacctcccacgcctggtgcggcctgaggtcgacgtcatgtgcaccgctttccatgacaatgaggagacttttctcaagaagtatctgtacgagatcgcccggaggcacccatacttttatgcaccggagctccttttcttcgctaagcggtacaaggcggcgttcactgaatgctgtcaggcagcagacaaggcagcatgcctcctgccgaaactggacgaacttcgcgacgagggtaaagcgtcgtccgccaagcagcgccttaagtgcgcctcgttgcagaagtttggtgaacgcgcattcaaagcgtgggccgtcgcaagactttcgcagcggttcccaaaagcggagtttgccgaggtgtccaaactggtcaccgacctgaccaaggtccacaccgagtgctgccacggcgatctgctcgaatgcgccgacgaccgggctgatctcgcaaagtacatttgcgagaaccaagactcgatctcgtcaaaactgaaggaatgctgcgagaagccgctgttggaaaagagccattgtatcgccgaagtggagaacgatgaaatgcctgctgatctgccaagcctcgccgcagactttgtggagagcaaagacgtgtgcaagaactacgccgaagcgaaggacgtgtttctcgggatgttcctctacgagtacgcgcgcaggcaccctgactactcagtggtcctgctgttgcggctggccaaaacttacgaaaccaccctcgaaaagtgctgcgcggctgccgatccacatgaatgctacgcaaaggtgttcgatgaatttaagcctctggtggaggaaccacagaacctgatcaagcaaaattgtgaactgtttgaacagctgggagagtacaaatttcagaatgccctgctggtcagatacactaagaaggtgccccaagtctccactccaaccctcgtggaggtgtcacggaatctcggcaaagtgggcagcaaatgctgtaagcacccggaagcaaagaggatgccctgcgctgaagattacctgtccgtggtgctgaatcagctttgtgtgctgcacgaaaagacgcctgtctccgaccgggtgaccaagtgctgtaccgaatcgctcgtgaatcgcagaccctgcttctccgctctcgaagtggacgaaacttacgtcccgaaggagttcaatgcggaaaccttcaccttccacgcggacatctgtaccctgagcgaaaaagagcggcagatcaagaaacagactgccctggtggaactggtgaagcacaagccgaaggcaacgaaggagcagctgaaggcggtgatggatgactttgcagccttcgtggaaaagtgttgcaaggcagatgataaagaaacctgtttcgcggaagaggggaagaagttggtggctgccagccaggccgctctcggactgggaggtggaggatcaggaggcggaggctccggaggaggaggctcggctcacaatggcgatcattgcccgctcggaccgggacgctgctgcagactgcataccgtccgcgcttccttggaagatctgggatgggcggattgggtgttgtcaccaagagaggtgcaagtgacgatgtgtatcggtgcgtgcccttcacagttccgcgctgcgaacatgcatgcccaaatcaagaccagcctgcaccggctgaagccggacactgtcccagctccatgttgcgtgcccgcatcgtacaacccgatggtgctcatccagaaaactgacactggagtctcactgcaaacgtacgacgatttgctcgccaaagattgccactgcatt

SEQ ID NO: 38 - protein, 712 aa, Artificial Sequence
  source: /note=“Description of Artificial Sequence Synthetic polypeptide”
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAHNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 39 - DNA, 2196 bp, Artificial Sequence
  source: /note=“Description of Artificial Sequence Synthetic polynucleotide”
atggaaaccgatactctgctgctgtgggtgcttcttctttgggtgccgggatcaaccggcgatgcccacaagtcggaggtggcccatcggtttaaggacctcggggaggagaacttcaaagccctggtcctcatcgccttcgcccaatacctccagcagtgtccattcgaagatcacgtgaagctcgtgaacgaagtgactgaatttgccaagacttgtgtcgcagacgaaagcgccgaaaactgcgacaagtcgttgcatactctcttcggggataagctgtgcactgtcgcaacccttagagagacttacggtgaaatggctgattgctgcgccaaacaagagccggagcgcaacgagtgcttcctccaacataaggacgacaaccccaacctcccacgcctggtgcggcctgaggtcgacgtcatgtgcaccgctttccatgacaatgaggagacttttctcaagaagtatctgtacgagatcgcccggaggcacccatacttttatgcaccggagctccttttcttcgctaagcggtacaaggcggcgttcactgaatgctgtcaggcagcagacaaggcagcatgcctcctgccgaaactggacgaacttcgcgacgagggtaaagcgtcgtccgccaagcagcgccttaagtgcgcctcgttgcagaagtttggtgaacgcgcattcaaagcgtgggccgtcgcaagactttcgcagcggttcccaaaagcggagtttgccgaggtgtccaaactggtcaccgacctgaccaaggtccacaccgagtgctgccacggcgatctgctcgaatgcgccgacgaccgggctgatctcgcaaagtacatttgcgagaaccaagactcgatctcgtcaaaactgaaggaatgctgcgagaagccgctgttggaaaagagccattgtatcgccgaagtggagaacgatgaaatgcctgctgatctgccaagcctcgccgcagactttgtggagagcaaagacgtgtgcaagaactacgccgaagcgaaggacgtgtttctcgggatgttcctctacgagtacgcgcgcaggcaccctgactactcagtggtcctgctgttgcggctggccaaaacttacgaaaccaccctcgaaaagtgctgcgcggctgccgatccacatgaatgctacgcaaaggtgttcgatgaatttaagcctctggtggaggaaccacagaacctgatcaagcaaaattgtgaactgtttgaacagctgggagagtacaaatttcagaatgccctgctggtcagatacactaagaaggtgccccaagtctccactccaaccctcgtggaggtgtcacggaatctcggcaaagtgggcagcaaatgctgtaagcacccggaagcaaagaggatgccctgcgctgaagattacctgtccgtggtgctgaatcagctttgtgtgctgcacgaaaagacgcctgtctccgaccgggtgaccaagtgctgtaccgaatcgctcgtgaatcgcagaccctgcttctccgctctcgaagtggacgaaacttacgtcccgaaggagttcaatgcggaaaccttcaccttccacgcggacatctgtaccctgagcgaaaaagagcggcagatcaagaaacagactgccctggtggaactggtgaagcacaagccgaaggcaacgaaggagcagctgaaggcggtgatggatgactttgcagccttcgtggaaaagtgttgcaaggcagatgataaagaaacctgtttcgcggaagaggggaagaagttggtggctgccagccaggccgctctcggactgggaggtggaggatcaggaggcggaggctccggaggaggaggctcggctcacgccggcgatcattgcccgctcggaccgggacgctgctgcagactgcataccgtccgcgcttccttggaagatctgggatgggcggattgggtgttgtcaccaagagaggtgcaagtgacgatgtgtatcggtgcgtgcccttcacagttccgcgctgcgaacatgcatgcccaaatcaagaccagcctgcaccggctgaagccggacactgtcccagctccatgttgcgtgcccgcatcgtacaacccgatggtgctcatccagaaaactgacactggagtctcactgcaaacgtacgacgatttgctcgccaaagattgccactgcatt

SEQ ID NO: 40 - protein, 712 aa, Artificial Sequence
  source: /note=“Description of Artificial Sequence Synthetic polypeptide”
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAHAGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 41 - DNA, 2196 bp, Artificial Sequence
  source: /note=“Description of Artificial Sequence Synthetic polynucleotide”
atggaaaccgatactctgctgctgtgggtgcttcttctttgggtgccgggatcaaccggcgatgcccacaagtcggaggtggcccatcggtttaaggacctcggggaggagaacttcaaagccctggtcctcatcgccttcgcccaatacctccagcagtgtccattcgaagatcacgtgaagctcgtgaacgaagtgactgaatttgccaagacttgtgtcgcagacgaaagcgccgaaaactgcgacaagtcgttgcatactctcttcggggataagctgtgcactgtcgcaacccttagagagacttacggtgaaatggctgattgctgcgccaaacaagagccggagcgcaacgagtgcttcctccaacataaggacgacaaccccaacctcccacgcctggtgcggcctgaggtcgacgtcatgtgcaccgctttccatgacaatgaggagacttttctcaagaagtatctgtacgagatcgcccggaggcacccatacttttatgcaccggagctccttttcttcgctaagcggtacaaggcggcgttcactgaatgctgtcaggcagcagacaaggcagcatgcctcctgccgaaactggacgaacttcgcgacgagggtaaagcgtcgtccgccaagcagcgccttaagtgcgcctcgttgcagaagtttggtgaacgcgcattcaaagcgtgggccgtcgcaagactttcgcagcggttcccaaaagcggagtttgccgaggtgtccaaactggtcaccgacctgaccaaggtccacaccgagtgctgccacggcgatctgctcgaatgcgccgacgaccgggctgatctcgcaaagtacatttgcgagaaccaagactcgatctcgtcaaaactgaaggaatgctgcgagaagccgctgttggaaaagagccattgtatcgccgaagtggagaacgatgaaatgcctgctgatctgccaagcctcgccgcagactttgtggagagcaaagacgtgtgcaagaactacgccgaagcgaaggacgtgtttctcgggatgttcctctacgagtacgcgcgcaggcaccctgactactcagtggtcctgctgttgcggctggccaaaacttacgaaaccaccctcgaaaagtgctgcgcggctgccgatccacatgaatgctacgcaaaggtgttcgatgaatttaagcctctggtggaggaaccacagaacctgatcaagcaaaattgtgaactgtttgaacagctgggagagtacaaatttcagaatgccctgctggtcagatacactaagaaggtgccccaagtctccactccaaccctcgtggaggtgtcacggaatctcggcaaagtgggcagcaaatgctgtaagcacccggaagcaaagaggatgccctgcgctgaagattacctgtccgtggtgctgaatcagctttgtgtgctgcacgaaaagacgcctgtctccgaccgggtgaccaagtgctgtaccgaatcgctcgtgaatcgcagaccctgcttctccgctctcgaagtggacgaaacttacgtcccgaaggagttcaatgcggaaaccttcaccttccacgcggacatctgtaccctgagcgaaaaagagcggcagatcaagaaacagactgccctggtggaactggtgaagcacaagccgaaggcaacgaaggagcagctgaaggcggtgatggatgactttgcagccttcgtggaaaagtgttgcaaggcagatgataaagaaacctgtttcgcggaagaggggaagaagttggtggctgccagccaggccgctctcggactgggaggtggaggatcaggaggcggaggctccggaggaggaggctcggctcgcgagggcgatcattgcccgctcggaccgggacgctgctgcagactgcataccgtccgcgcttccttggaagatctgggatgggcggattgggtgttgtcaccaagagaggtgcaagtgacgatgtgtatcggtgcgtgcccttcacagttccgcgctgcgaacatgcatgcccaaatcaagaccagcctgcaccggctgaagccggacactgtcccagctccatgttgcgtgcccgcatcgtacaacccgatggtgctcatccagaaaactgacactggagtctcactgcaaacgtacgacgatttgctcgccaaagattgccactgcatt

SEQ ID NO: 42 - protein, 712 aa, Artificial Sequence
  source: /note=“Description of Artificial Sequence Synthetic polypeptide”
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAREGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 43 - protein, 7 aa, Homo sapiens
RGRRRAR

SEQ ID NO: 44 - protein, 112 aa, Homo sapiens; identical to SEQ ID NO: 11

SEQ ID NO: 45 - protein, 585 aa, Homo sapiens [sequence omitted]

SEQ ID NO: 46 - protein, 100 aa, Artificial Sequence
  source: /note=“Description of Artificial Sequence Synthetic polypeptide”
  VARIANT (6)..(100): /replace=“ ”
  misc_feature (1)..(100): /note=“This sequence may encompass 1-20 ′Gly Gly Gly Gly Ser′ repeating units, wherein some positions may be absent”
  misc_feature (1)..(100): /note=“Variant residues given in the sequence have no preference with respect to those in the annotations for variant positions”
  source: /note=“See specification as filed for detailed description of substitutions and preferred embodiments”
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 47 - protein, 15 aa, Artificial Sequence
  source: /note=“Description of Artificial Sequence Synthetic peptide”
GGGGSGGGGSGGGGS

SEQ ID NO: 48 - protein, 100 aa, Artificial Sequence
  source: /note=“Description of Artificial Sequence Synthetic polypeptide”
  VARIANT (6)..(100): /replace=“ ”
  misc_feature (1)..(100): /note=“This sequence may encompass 1-20 ′Gly Pro Pro Gly Ser′ repeating units, wherein some positions may be absent”
  misc_feature (1)..(100): /note=“Variant residues given in the sequence have no preference with respect to those in the annotations for variant positions”
  source: /note=“See specification as filed for detailed description of substitutions and preferred embodiments”
GPPGSGPPGSGPPGSGPPGSGPPGSGPPGSGPPGSGPPGSGPPGSGPPGSGPPGSGPPGSGPPGSGPPGSGPPGSGPPGSGPPGSGPPGSGPPGSGPPGS

SEQ ID NO: 49 - protein, 5 aa, Artificial Sequence
  source: /note=“Description of Artificial Sequence Synthetic peptide”
GPPGS

SEQ ID NO: 50 - protein, 100 aa, Artificial Sequence
  source: /note=“Description of Artificial Sequence Synthetic polypeptide”
  VARIANT (6)..(100): /replace=“ ”
  misc_feature (1)..(100): /note=“This sequence may encompass 1-20 ′Gly Ser Gly Gly Gly′ repeating units, wherein some positions may be absent”
  misc_feature (1)..(100): /note=“Variant residues given in the sequence have no preference with respect to those in the annotations for variant positions”
  source: /note=“See specification as filed for detailed description of substitutions and preferred embodiments”
GSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGG

SEQ ID NO: 51 - protein, 5 aa, Artificial Sequence
  source: /note=“Description of Artificial Sequence Synthetic peptide”
GSGGG

SEQ ID NO: 52 - protein, 4 aa, Artificial Sequence
  source: /note=“Description of Artificial Sequence Synthetic peptide”
GSGG

SEQ ID NO: 53 - protein, 80 aa, Artificial Sequence
  source: /note=“Description of Artificial Sequence Synthetic polypeptide”
  VARIANT (5)..(80): /replace=“ ”
  misc_feature (1)..(80): /note=“This sequence may encompass 1-20 ′Ser Gly Gly Gly′ repeating units, wherein some positions may be absent”
  misc_feature (1)..(80): /note=“Variant residues given in the sequence have no preference with respect to those in the annotations for variant positions”
  source: /note=“See specification as filed for detailed description of substitutions and preferred embodiments”
SGGGSGGGSGGGSGGGSGGGSGGGSGGGSGGGSGGGSGGGSGGGSGGGSGGGSGGGSGGGSGGGSGGGSGGGSGGGSGGG

SEQ ID NO: 54 - protein, 5 aa, Artificial Sequence
  source: /note=“Description of Artificial Sequence Synthetic peptide”
GGGGS

SEQ ID NO: 55 - protein, 4 aa, Artificial Sequence
  source: /note=“Description of Artificial Sequence Synthetic peptide”
GGGS

SEQ ID NO: 56 - protein, 3 aa, Artificial Sequence
  source: /note=“Description of Artificial Sequence Synthetic peptide”
GGS

SEQ ID NO: 57 - protein, 2 aa, Artificial Sequence
  source: /note=“Description of Artificial Sequence Synthetic peptide”
GS

SEQ ID NO: 58 - protein, 5 aa, Artificial Sequence
  source: /note=“Description of Artificial Sequence Synthetic peptide”
SGGGG

SEQ ID NO: 59 - protein, 4 aa, Artificial Sequence
  source: /note=“Description of Artificial Sequence Synthetic peptide”
SGGG

SEQ ID NO: 60 - protein, 3 aa, Artificial Sequence
  source: /note=“Description of Artificial Sequence Synthetic peptide”
SGG

SEQ ID NO: 61 - protein, 11 aa, Artificial Sequence
  source: /note=“Description of Artificial Sequence Synthetic peptide”
GGGSGGGGSGS

SEQ ID NO: 62 - protein, 5 aa, Artificial Sequence
  source: /note=“Description of Artificial Sequence Synthetic peptide”
GGGGA

SEQ ID NO: 63 - protein, 4 aa, Artificial Sequence
  source: /note=“Description of Artificial Sequence Synthetic peptide”
GGGA

SEQ ID NO: 64 - protein, 100 aa, Artificial Sequence
  source: /note=“Description of Artificial Sequence Synthetic polypeptide”
  VARIANT (6)..(100): /replace=“ ”
  misc_feature (1)..(100): /note=“This sequence may encompass 1-20 ′Glu Ala Ala Ala Lys′ repeating units, wherein some positions may be absent”
  misc_feature (1)..(100): /note=“Variant residues given in the sequence have no preference with respect to those in the annotations for variant positions”
  source: /note=“See specification as filed for detailed description of substitutions and preferred embodiments”
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 65 - DNA, 2196 bp, Artificial Sequence
  source: /note=“Description of Artificial Sequence Synthetic polynucleotide”
atggagactgataccctgctcctctgggtgctgcttctctgggtccctggctcaaccggcgaagcccacaagtccgagatcgcccatcgctataatgctcttggagaacagcatttcaagggactggtgctgattgccttctcccagtacctccaaaaggccagctatgatgagcacgccaagctcgtccaagaagtcaccgactttgctaagacttgtgtggccgacgaaagcgctgccaattgcgataagtcactccatactctcttcggggacaagctgtgcgctattcccaacctccgcgagaattacggtgagctggccgactgttgcaccaaacaggagccagagcggaacgagtgcttccttcaacacaaagatgacaatccttcactgcctcctttcgaacggcccgaggcagaggcaatgtgcactagcttcaaggagaacccaaccaccttcatgggacactacctccatgaggtcgctagacggcatccctacttctatgccccagagcttctgtattatgcagaacagtacaatgagatcctgacccagtgctgtgctgaggctgataaggagagctgcctgaccccaaagctcgacggagtgaaggaaaaggctcttgtgtccagcgtgcggcagcgcatgaagtgctcttcaatgcagaagtttggggagcgcgccttcaaagcctgggccgtggccagactgtcccagacctttcctaatgctgactttgccgagatcaccaagctcgctactgacctgaccaaggtcaacaaagagtgttgccacggagatctgctcgaatgcgccgacgaccgcgctgagcttgctaagtacatgtgcgaaaaccaggcaaccatttctagcaagctgcagacctgttgtgataagcctctgctgaagaaagcccattgcctcagcgaggtcgaacatgacactatgccggcagacctccccgctatcgccgctgacttcgtggaggaccaagaagtgtgcaagaattacgccgaggctaaggacgtgttccttggtactttcctctacgagtatagccggaggcaccctgactacagcgtgtctcttctgcttcggctcgccaagaagtacgaagccaccctcgaaaaatgctgcgccgaagcaaatccgccagcttgttacgggactgtgctggctgagtttcagcccctggtggaagagcccaagaacctcgtcaagaccaactgcgacctttacgagaaactgggtgaatacgggtttcagaatgccattctggtgcggtacacccagaaggcaccacaagtgtccaccccaacccttgtcgaggcagcccgcaaccttggacgcgtcgggaccaagtgttgtaccctgcccgaggaccaacgcctgccctgcgtcgaggactaccttagcgccattctgaacagagtctgtctgctccatgaaaagacccctgtgtctgagcacgtgaccaagtgctgttcaggctcactggtggagaggaggccttgcttttctgccctgaccgtggacgaaacctacgtgcccaaggagttcaaagctgaaaccttcactttccattcagacatctgtaccctccccgaaaaggaaaagcaaatcaagaagcagaccgcccttgctgaactggtgaagcacaagccaaaggccaccgccgaacaactcaagactgtgatggacgacttcgctcagttcctcgacacttgctgcaaagccgccgacaaagatacctgtttctcaaccgaggggccgaacctggtgactagagccaaggacgccctggccggaggaggtggttctggcggtggtggttccggcggaggaggatctgccaggaatggagatcactgcccactcggaccgggacggtgttgtcgcctgcacactgtgcgcgcatctcttgaggatctgggatgggctgattgggtgctctctcccagagaggtgcaagtcaccatgtgcattggcgcctgcccctccaggttcagggcagctaacatgcatgctcagatcaagactagcctgcacaggctgaagcccgacactgtccctgccccatgttgtgtgccggcctcctataacccaatggtcctgatccaaaagaccgataccggagtgtcacttcagacttacgacgatctgcttgcaaaagactgccattgcatctga

SEQ ID NO: 66 - protein, 711 aa, Artificial Sequence
  source: /note=“Description of Artificial Sequence Synthetic polypeptide”
EAHKSEIAHRYNALGEQHFKGLVLIAFSQYLQKASYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRAKDALAGGGGSGGGGSGGGGSARNGDHCPLGPGRCCRLHTVRASLEDLGWADWVLSPREVQVTMCIGACPSRFRAANMHAQIKTSLHRLKPDTVPAPCCVPASYNPMVLIQKTDTGVSLQTYDDLLAKDCHCI

SEQ ID NO: 67 - DNA, 2196 bp, Artificial Sequence
  source: /note=“Description of Artificial Sequence Synthetic polynucleotide”
atggagactgataccctgctcctctgggtgctgcttctctgggtccctggctcaaccggcgaagcccacaagtccgagatcgcccatcgctataatgctcttggagaacagcatttcaagggactggtgctgattgccttctcccagtacctccaaaaggccagctatgatgagcacgccaagctcgtccaagaagtcaccgactttgctaagacttgtgtggccgacgaaagcgctgccaattgcgataagtcactccatactctcttcggggacaagctgtgcgctattcccaacctccgcgagaattacggtgagctggccgactgttgcaccaaacaggagccagagcggaacgagtgcttccttcaacacaaagatgacaatccttcactgcctcctttcgaacggcccgaggcagaggcaatgtgcactagcttcaaggagaacccaaccaccttcatgggacactacctccatgaggtcgctagacggcatccctacttctatgccccagagcttctgtattatgcagaacagtacaatgagatcctgacccagtgctgtgctgaggctgataaggagagctgcctgaccccaaagctcgacggagtgaaggaaaaggctcttgtgtccagcgtgcggcagcgcatgaagtgctcttcaatgcagaagtttggggagcgcgccttcaaagcctgggccgtggccagactgtcccagacctttcctaatgctgactttgccgagatcaccaagctcgctactgacctgaccaaggtcaacaaagagtgttgccacggagatctgctcgaatgcgccgacgaccgcgctgagcttgctaagtacatgtgcgaaaaccaggcaaccatttctagcaagctgcagacctgttgtgataagcctctgctgaagaaagcccattgcctcagcgaggtcgaacatgacactatgccggcagacctccccgctatcgccgctgacttcgtggaggaccaagaagtgtgcaagaattacgccgaggctaaggacgtgttccttggtactttcctctacgagtatagccggaggcaccctgactacagcgtgtctcttctgcttcggctcgccaagaagtacgaagccaccctcgaaaaatgctgcgccgaagcaaatccgccagcttgttacgggactgtgctggctgagtttcagcccctggtggaagagcccaagaacctcgtcaagaccaactgcgacctttacgagaaactgggtgaatacgggtttcagaatgccattctggtgcggtacacccagaaggcaccacaagtgtccaccccaacccttgtcgaggcagcccgcaaccttggacgcgtcgggaccaagtgttgtaccctgcccgaggaccaacgcctgccctgcgtcgaggactaccttagcgccattctgaacagagtctgtctgctccatgaaaagacccctgtgtctgagcacgtgaccaagtgctgttcaggctcactggtggagaggaggccttgcttttctgccctgaccgtggacgaaacctacgtgcccaaggagttcaaagctgaaaccttcactttccattcagacatctgtaccctccccgaaaaggaaaagcaaatcaagaagcagaccgcccttgctgaactggtgaagcacaagccaaaggccaccgccgaacaactcaagactgtgatggacgacttcgctcagttcctcgacacttgctgcaaagccgccgacaaagatacctgtttctcaaccgaggggccgaacctggtgactagagccaaggacgccctggccggaggaggtggttctggcggtggtggttccggcggaggaggatctgccaggaatggagatcactgcccactcggaccgggacggtgttgtcgcctgcacactgtgcgcgcatctcttgaggatctgggatgggctgattgggtgctctctcccagagaggtgcaagtcaccatgtgcattggcgcctgcccctcccaattcagggcagctaacatgcatgctcagatcaagactagcctgcacaggctgaagcccgacactgtccctgccccatgttgtgtgccggccaggtataacccaatggtcctgatccaaaagaccgataccggagtgtcacttcagacttacgacgatctgcttgcaaaagactgccattgcatctga

SEQ ID NO: 68 - protein, 711 aa, Artificial Sequence
  source: /note=“Description of Artificial Sequence Synthetic polypeptide”
EAHKSEIAHRYNALGEQHFKGLVLIAFSQYLQKASYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRAKDALAGGGGSGGGGSGGGGSARNGDHCPLGPGRCCRLHTVRASLEDLGWADWVLSPREVQVTMCIGACPSQFRAANMHAQIKTSLHRLKPDTVPAPCCVPARYNPMVLIQKTDTGVSLQTYDDLLAKDCHCI

SEQ ID NO: 69 - DNA, 2196 bp, Artificial Sequence
  source: /note=“Description of Artificial Sequence Synthetic polynucleotide”
atggagactgataccctgctcctctgggtgctgcttctctgggtccctggctcaaccggcgaagcccacaagtccgagatcgcccatcgctataatgctcttggagaacagcatttcaagggactggtgctgattgccttctcccagtacctccaaaaggccagctatgatgagcacgccaagctcgtccaagaagtcaccgactttgctaagacttgtgtggccgacgaaagcgctgccaattgcgataagtcactccatactctcttcggggacaagctgtgcgctattcccaacctccgcgagaattacggtgagctggccgactgttgcaccaaacaggagccagagcggaacgagtgcttccttcaacacaaagatgacaatccttcactgcctcctttcgaacggcccgaggcagaggcaatgtgcactagcttcaaggagaacccaaccaccttcatgggacactacctccatgaggtcgctagacggcatccctacttctatgccccagagcttctgtattatgcagaacagtacaatgagatcctgacccagtgctgtgctgaggctgataaggagagctgcctgaccccaaagctcgacggagtgaaggaaaaggctcttgtgtccagcgtgcggcagcgcatgaagtgctcttcaatgcagaagtttggggagcgcgccttcaaagcctgggccgtggccagactgtcccagacctttcctaatgctgactttgccgagatcaccaagctcgctactgacctgaccaaggtcaacaaagagtgttgccacggagatctgctcgaatgcgccgacgaccgcgctgagcttgctaagtacatgtgcgaaaaccaggcaaccatttctagcaagctgcagacctgttgtgataagcctctgctgaagaaagcccattgcctcagcgaggtcgaacatgacactatgccggcagacctccccgctatcgccgctgacttcgtggaggaccaagaagtgtgcaagaattacgccgaggctaaggacgtgttccttggtactttcctctacgagtatagccggaggcaccctgactacagcgtgtctcttctgcttcggctcgccaagaagtacgaagccaccctcgaaaaatgctgcgccgaagcaaatccgccagcttgttacgggactgtgctggctgagtttcagcccctggtggaagagcccaagaacctcgtcaagaccaactgcgacctttacgagaaactgggtgaatacgggtttcagaatgccattctggtgcggtacacccagaaggcaccacaagtgtccaccccaacccttgtcgaggcagcccgcaaccttggacgcgtcgggaccaagtgttgtaccctgcccgaggaccaacgcctgccctgcgtcgaggactaccttagcgccattctgaacagagtctgtctgctccatgaaaagacccctgtgtctgagcacgtgaccaagtgctgttcaggctcactggtggagaggaggccttgcttttctgccctgaccgtggacgaaacctacgtgcccaaggagttcaaagctgaaaccttcactttccattcagacatctgtaccctccccgaaaaggaaaagcaaatcaagaagcagaccgcccttgctgaactggtgaagcacaagccaaaggccaccgccgaacaactcaagactgtgatggacgacttcgctcagttcctcgacacttgctgcaaagccgccgacaaagatacctgtttctcaaccgaggggccgaacctggtgactagagccaaggacgccctggccggaggaggtggttctggcggtggtggttccggcggaggaggatctgccaggaatggagatcactgcccactcggaccgggacggtgttgtcgcctgcacactgtgcgcgcatctcttgaggatctgggatgggctgattgggtgctctctcccagagaggtgcaagtcaccatgtgcattggcgcctgcccctcccaattcagggcagctaacatgcatgctcagatcaagactagcctgcacaggctgaagcccgacactgtccctgccccatgttgtgtgccggcctcctataacccaatggtcctgatccaaaagaccaggaccggagtgtcacttcagacttacgacgatctgcttgcaaaagactgccattgcatctga

SEQ ID NO: 70 - protein, 711 aa, Artificial Sequence
  source: /note=“Description of Artificial Sequence Synthetic polypeptide”
EAHKSEIAHRYNALGEQHFKGLVLIAFSQYLQKASYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRAKDALAGGGGSGGGGSGGGGSARNGDHCPLGPGRCCRLHTVRASLEDLGWADWVLSPREVQVTMCIGACPSQFRAANMHAQIKTSLHRLKPDTVPAPCCVPASYNPMVLIQKTRTGVSLQTYDDLLAKDCHCI

SEQ ID NO: 71 - DNA, 2196 bp, Artificial Sequence
  source: /note=“Description of Artificial Sequence Synthetic polynucleotide”
atggagactgataccctgctcctctgggtgctgcttctctgggtccctggctcaaccggcgaagcccacaagtccgagatcgcccatcgctataatgctcttggagaacagcatttcaagggactggtgctgattgccttctcccagtacctccaaaaggccagctatgatgagcacgccaagctcgtccaagaagtcaccgactttgctaagacttgtgtggccgacgaaagcgctgccaattgcgataagtcactccatactctcttcggggacaagctgtgcgctattcccaacctccgcgagaattacggtgagctggccgactgttgcaccaaacaggagccagagcggaacgagtgcttccttcaacacaaagatgacaatccttcactgcctcctttcgaacggcccgaggcagaggcaatgtgcactagcttcaaggagaacccaaccaccttcatgggacactacctccatgaggtcgctagacggcatccctacttctatgccccagagcttctgtattatgcagaacagtacaatgagatcctgacccagtgctgtgctgaggctgataaggagagctgcctgaccccaaagctcgacggagtgaaggaaaaggctcttgtgtccagcgtgcggcagcgcatgaagtgctcttcaatgcagaagtttggggagcgcgccttcaaagcctgggccgtggccagactgtcccagacctttcctaatgctgactttgccgagatcaccaagctcgctactgacctgaccaaggtcaacaaagagtgttgccacggagatctgctcgaatgcgccgacgaccgcgctgagcttgctaagtacatgtgcgaaaaccaggcaaccatttctagcaagctgcagacctgttgtgataagcctctgctgaagaaagcccattgcctcagcgaggtcgaacatgacactatgccggcagacctccccgctatcgccgctgacttcgtggaggaccaagaagtgtgcaagaattacgccgaggctaaggacgtgttccttggtactttcctctacgagtatagccggaggcaccctgactacagcgtgtctcttctgcttcggctcgccaagaagtacgaagccaccctcgaaaaatgctgcgccgaagcaaatccgccagcttgttacgggactgtgctggctgagtttcagcccctggtggaagagcccaagaacctcgtcaagaccaactgcgacctttacgagaaactgggtgaatacgggtttcagaatgccattctggtgcggtacacccagaaggcaccacaagtgtccaccccaacccttgtcgaggcagcccgcaaccttggacgcgtcgggaccaagtgttgtaccctgcccgaggaccaacgcctgccctgcgtcgaggactaccttagcgccattctgaacagagtctgtctgctccatgaaaagacccctgtgtctgagcacgtgaccaagtgctgttcaggctcactggtggagaggaggccttgcttttctgccctgaccgtggacgaaacctacgtgcccaaggagttcaaagctgaaaccttcactttccattcagacatctgtaccctccccgaaaaggaaaagcaaatcaagaagcagaccgcccttgctgaactggtgaagcacaagccaaaggccaccgccgaacaactcaagactgtgatggacgacttcgctcagttcctcgacacttgctgcaaagccgccgacaaagatacctgtttctcaaccgaggggccgaacctggtgactagagccaaggacgccctggccggaggaggtggttctggcggtggtggttccggcggaggaggatctgccaggaatggagatcactgcccactcggaccgggacggtgttgtcgcctgcacactgtgcgcgcatctcttgaggatctgggatgggctgattgggtgctctctcccagagaggtgcaagtcaccatgtgcattggcgcctgcccctcccaattcagggcagctaacatgcatgctcagatcaagactagcctgcacaggctgaagcccgacactgtccctgccccatgttgtgtgccggcctcctataacccaatggtcctgatccaaaagaccgataccggagtgtcaaggcagacttacgacgatctgcttgcaaaagactgccattgcatctga

SEQ ID NO: 72 - protein, 711 aa, Artificial Sequence
  source: /note=“Description of Artificial Sequence Synthetic polypeptide”
EAHKSEIAHRYNALGEQHFKGLVLIAFSQYLQKASYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRAKDALAGGGGSGGGGSGGGGSARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSRQTYDDLLAKDCHCI

SEQ ID NO: 73 - protein, 4 aa, Artificial Sequence
  source: /note=“Description of Artificial Sequence Synthetic peptide”
GGGG

SEQ ID NO: 74 - protein, 5 aa, Artificial Sequence
  source: /note=“Description of Artificial Sequence Synthetic peptide”
GGGGG

SEQ ID NO: 75 - protein, 6 aa, Artificial Sequence
  source: /note=“Description of Artificial Sequence Synthetic 6xHis tag”
HHHHHH

SEQ ID NO: 76 - protein, 8 aa, Artificial Sequence
  source: /note=“Description of Artificial Sequence Synthetic 8xHis tag”
HHHHHHHH

SEQ ID NO: 77 - protein, 20 aa, Artificial Sequence
  source: /note=“Description of Artificial Sequence Synthetic peptide”
  VARIANT (6)..(20): /replace=“ ”
  misc_feature (1)..(20): /note=“This sequence may encompass 1-4 ′Gly Gly Gly Gly Ser′ repeating units, wherein some positions may be absent”
  misc_feature (1)..(20): /note=“Variant residues given in the sequence have no preference with respect to those in the annotations for variant positions”
  source: /note=“See specification as filed for detailed description of substitutions and preferred embodiments”
GGGGSGGGGSGGGGSGGGGS

SEQ ID NO: 78 - protein, 20 aa, Artificial Sequence
  source: /note=“Description of Artificial Sequence Synthetic peptide”
  VARIANT (6)..(20): /replace=“ ”
  misc_feature (1)..(20): /note=“This sequence may encompass 1-4 ′Gly Pro Pro Gly Ser′ repeating units, wherein some positions may be absent”
  misc_feature (1)..(20): /note=“Variant residues given in the sequence have no preference with respect to those in the annotations for variant positions”
  source: /note=“See specification as filed for detailed description of substitutions and preferred embodiments”
GPPGSGPPGSGPPGSGPPGS